SEQUENCE LISTING (37 sequences)

SEQ ID NO: 1 - protein, 305 aa, synthetic construct
MPQQLSPINIETKKAISDARLKTLDIHYNESKPTTIQNTGKLVRINFKGGYISGGFLPNEYVLSTHIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYSGEIIRAATTSPVRENYFMKWLSDLREACFSYYQKYIEGNKTFAIIAIVFVFILTAILFLMSQRYSREKQN

SEQ ID NO: 2 - protein, 305 aa, synthetic construct
MPQQLSPINIETKKAISNARLKPLDIHYNESKPTTIQNTGKLVRINFKGGYISGGFLPNEYVLSTSLRIYWGKEDDYGSNHLIDVYKYSGEINLVHWNKKKYSSYEEAKKHDDGLIIISIFLQVSDHKNVYFQKIVNQLDSIRSTNTSAPFDSVFYLDNLLPSKLDYFTYLGTTINHSADAVWIIFPTPINIHSDQLSKFRTLLSSSNHDGKPHYITENYRNPYKLNDDTQVYYSGEIIRAATTSPARENYFMRWLSDLRETCFSYYQKYIEGNKTFAIIAIVFVFILTAILFFMSQRYSREKQN

SEQ ID NO: 3 - protein, 250 aa, synthetic construct
MGAAASIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMIIATTDIQN

SEQ ID NO: 4 - protein, 250 aa, synthetic construct
MGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMVIATTDMQN

SEQ ID NO: 5 - protein, 317 aa, synthetic construct
MKTISVVTLLCVLPAVVYSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYHIIIMALTIMGVIFLISIIVLVCSCDKNNDQYKFHKLLP

SEQ ID NO: 6 - protein, 317 aa, synthetic construct
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 7 - protein, 181 aa, synthetic construct
MMTPENDEEQTSVFSATVYGDKIQGKNKRKRVIGLCIRISMVISLLSMITMSAFLIVRLNQCMSANKAAITDSAVAVAAASSTHRKVVSSTTQYDHKESCNGLYYQGSCYILHSDYKSFEDAKANCAAESSTLPNKSDVLTTWLIDYVEDTWGSDGNPITKTTSDYQDSDVSQEVRKYFCT

SEQ ID NO: 8 - protein, 184 aa, synthetic construct
MMTPENDEEQTSVFSATVYRDKIQGKNKRKRVIGLCIRISMVISLLSMITMSAFLIVRLNQCMSANEAAITDAAVAVAAASSTHRKVASSTTQYDHKESCGLYYQGSCYILHSDYQLFSDAKANCTAESSTLPNKSDVLTTWLIDYVKDTWGSDGNPITKTTSDYQDSDVSQEVRKYFCVKTMN

SEQ ID NO: 9 - protein, 110 aa, synthetic construct
MDGTLFPGDDDLAIPATEFFSTKAAKNPETKREAIVKAYGDDNEETLKQRLTNLEKKITNITTKFEQIEKCCKRNDEVLFRLENHAETLRAAMISLAKKIDVQTGRHPYE

SEQ ID NO: 10 - protein, 110 aa, synthetic construct
MDGTLFPGDDDLAIPATEFFSTTAAKKPEAKREAIVKADEDDNEETLKQRLTNLEKKITNVTTKFEQIEKCCKRNDEVLFRLENHAETLRAAMISLAKKIDVQTGRRPYE

SEQ ID NO: 11 - protein, 51 aa, synthetic construct
PSSQPTIPIMGIVAGLAVLVVLAVLGAVVTAMMCGSGKKKKKKQKTKCVIM

SEQ ID NO: 12 - protein, 115 aa, synthetic construct
SPDVDLGDISGINASVVNIQKEIDRLNEVAKNLNESLIDLQELGKYEQYIKWPWYIWLGFIAGLIAIVMVTIMLGSCCMTSCCSCLKGCCSCGSCCKFDEDDSEPVLKGVKLHYT

SEQ ID NO: 13 - protein, 84 aa, synthetic construct
DKWASLWNWFNITNWLWYIKLFIMIIGGLVGLRTVCAVLSIVGSNRVRQGYSPLSFQTRLPNPRGPGRPEETEGEGGERDRDRS

SEQ ID NO: 14 - protein, 225 aa, synthetic construct
TTTPAPRPPTPAPTIASQPLSLRPEACRPAAGGAVHTRGLDFACDIYIWAPLAGTCGVLLLSLVITLYCGSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 271 aa, synthetic construct
QEFPSPQTYWKEITASVTVALLLVMLLIIVCHGSKKPNQPSRPSNTASKLERDSNADRETINLKELEPQIASAKPNAECGWDVWYCFHLCLAWLPLLARSRRSAQTLPYDAFVVFDKAQSAVADWVYNELRVRLEERRGRRALRLCLEDRDWLPGQTLFENLWASIYGSRKTLFVLAHTDRVSGLLRTSFLLAQQRLLEDRKDVVVLVILRPDAHRSRYVRLRQRLCRQSVLFWPQQPNGQGGFWAQLSTALTRDNRHFYNQNFCRGPTAE

SEQ ID NO: 16 - protein, 73 aa, synthetic construct
SGEIIRAATTSPVRENYFMKWLSDLREACFSYYQKYIEGNKTFAIIAIVFVFILTAILFLMGSSQRYSREKQN

SEQ ID NO: 17 - protein, 69 aa, synthetic construct
PSSQPTIPIMGIVAGLAVLVVLAVLGAVVTAMMCGSRRKSSGGKGGSCSQAACSNSAQGSDESLITCKA

SEQ ID NO: 18 - protein, 146 aa, synthetic construct
PSSQPTIPIMGIVAGLAVLVVLAVLGAVVTAMMCGSALPGNPDHREMGETLPEEVGEYRQPSGGSVPVSPGPPSGLEPTSSSPYGGGSFNSSINNIHEMEIQLKDALEKNQQWLVYDQQREVYVKGLLAKIFELEKKTETAAHSLP

SEQ ID NO: 19 - protein, 117 aa, synthetic construct
PSSQPTIPIMGIVAGLAVLVVLAVLGAVVTAMMCGSGGGSFNSSINNIHEMEIQLKDALEKNQQWLVYDQQREVYVKGLLAKIFELEKKTETAAHSLPGGGSGKKKKKKQKTKCVIM

SEQ ID NO: 20 - protein, 21 aa, synthetic construct
MALPVTALLLPLALLLHAARP

SEQ ID NO: 21 - protein, 13 aa, synthetic construct
MFVFLVLLPLVSS

SEQ ID NO: 22 - protein, 23 aa, synthetic construct
MDAMKRGLCCVLLLCGAVFVSPS

SEQ ID NO: 23 - protein, 20 aa, synthetic construct
MYRMQLLSCIALSLALVTNS

SEQ ID NO: 24 - protein, 18 aa, synthetic construct
MKWVTFISLLFLFSSAYS

SEQ ID NO: 25 - protein, 30 aa, synthetic construct
MKVMGTKKNYQHLWRWGIMLLGMLMMSSAA

SEQ ID NO: 26 - protein, 21 aa, synthetic construct
MWWRLWWLLLLLLLLWPMVWA

SEQ ID NO: 27 - protein, 22 aa, synthetic construct
MDMRVPAQLLGLLLLWLRGARC

SEQ ID NO: 28 - protein, 79 aa, synthetic construct
MEGGDQSEEEPRERSQAGGMGTLWSQESTPEERLPVEGSRPWAVARRVLTAILILGLLLCFSVLLFYNFQNCGPRPCET

SEQ ID NO: 29 - protein, 66 aa, synthetic construct
MVNDRWKTMGGAAQLEDRPRDKPQRPSCGYVLCTVLLALAVLLAVAVTGAVLFLNHAHAPGTAPPP

SEQ ID NO: 30 - protein, 71 aa, synthetic construct
AKGFYISKSLGILGILLGVAAVCTIIALSVVYSQEKNKNANSSPVASTTPSASATTNPASATTLDQSKAWN

SEQ ID NO: 31 - protein, 64 aa, synthetic construct
MTKEYQDLQHLDNEESDHHQLRKGPPPPQPLLQRLCSGPRLLLLSLGLSLLLLVVVCVIGSQNS

SEQ ID NO: 32 - protein, 56 aa, synthetic construct
MANCEFSPVSGDKPCCRLSRRAQLCLGVSILVLILVVVLAVVVPRWRQQWSGPGTT

SEQ ID NO: 33 - protein, 61 aa, synthetic construct
MIETYNQTSPRSAATGLPISMKIFMYLLTVFLITQMIGSALFAVYLHRRLDKIEDERNLHE

SEQ ID NO: 34 - protein, 62 aa, synthetic construct
MINPELRDGRADGFIHRIVPKLIQNWKIGLMCFLSIIITTVCIIMIATWSKHAKPVACSGDW

SEQ ID NO: 35 - protein, 74 aa, synthetic construct
MKQSPALAPEERCRRAGSPKPVLRADDNNMGNGCSQKLATANLLRFLLLVLIPCICALVLLLVILLSYVGTLQK

SEQ ID NO: 36 - protein, 50 aa, synthetic construct
MARGAEGGRGDAGWGLRGALAAVALLSALNAAGTVFALCQWRGLSSALRA

SEQ ID NO: 37 - protein, 1264 aa, synthetic construct
MDGTLFPGDDDLAIPATEFFSTKAAKNPETKREAIVKAYGDDNEETLKQRLTNLEKKITNITTKFEQIEKCCKRNDEVLFRLENHAETLRAAMISLAKKIDVQTGRHPYESGSGEGRGSLLTCGDVEENPGPTSKLMMTPENDEEQTSVFSATVYGDKIQGKNKRKRVIGLCIRISMVISLLSMITMSAFLIVRLNQCMSANKAAITDSAVAVAAASSTHRKVVSSTTQYDHKESCNGLYYQGSCYILHSDYKSFEDAKANCAAESSTLPNKSDVLTTWLIDYVEDTWGSDGNPITKTTSDYQDSDVSQEVRKYFCTSGSGATNFSLLKQAGDVEENPGPSRMGAAASIQTTVNTLSERISSKLEQEANASAQTKCDIEIGNFYIRQNHGCNITVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMIIATTDIQNSGSGQCTNYALLKLAGDVESNPGPSGMPQQLSPINIETKKAISDARLKTLDIHYNESKPTTIQNTGKLVRINFKGGYISGGFLPNEYVLSTIHIYWGKEDDYGSNHLIDVYKYSGEINLVHWNKKKYSSYEEAKKHDDGIIIIAIFLQVSDHKNVYFQKIVNQLDSIRSANMSAPFDSVFYLDNLLPSTLDYFTYLGTTINHSADAAWIIFPTPINIHSDQLSKFRTLLSSSNHEGKPHYITENYRNPYKLNDDTQVYYSGEIIRAATTSPVRENYFMKWLSDLREACFSYYQKYIEGNKTFAIIAIVFVFILTAILFLMSQRYSREKQNSGSGATNFSLLKQAGDVEENPGPPRMKTISVVTLLCVLPAVVYSTCTVPTMNNAKLTSTETSFNDKQKVTFTCDSGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGVSYISCTANSWNVIPSCQQKCDIPSLSNGLISGSTFSIGGVIHLSCKSGFTLTGSPSSTCIDGKWNPILPTCVRSNEEFDPVDDGPDDETDLSKLSKDVVQYEQEIESLEATYHIIIMALTIMGVIFLISIIVLVCSCDKNNDQYKFHKLLP

(19) United States
(12) Patent Application Publication
(10) Pub. No.: US 2026/0166138 A1
(43) Pub. Date: Jun. 18, 2026

(54) Compositions and Methods for Enhancement of mRNA Vaccine Performance and Vaccination against Mpox

(71) Applicant: Yale University, New Haven, CT (US)

(72) Inventors: Sidi Chen, New Haven, CT (US); Zhenhao Fang, New Haven, CT (US)

(21) Appl. No.: 19/127,070

(22) PCT Filed: Nov. 27, 2023
(86) PCT No.: PCT/US2023/081090

§ 371 Date: May 2, 2025

Related U.S. Application Data
(60) Provisional application No. 63/428,325, filed on Nov. 28, 2022.

(51) Int. Cl. A61K 39/275; A61K 39/00; A61P 37/04; C07K 14/005

(52) U.S. Cl. CPC A61K 39/275; A61P 37/04; C07K 14/005; A61K 2039/53; A61K 2039/6018; A61K 2039/70; C12N 2710/24122; C12N 2710/24134; C12N 2710/24152

(57) ABSTRACT

The current disclosure includes a modular vaccine platform. Also included are monkeypox vaccines that protect against pathogenic monkeypox (Mpox) species, as well as their variants. The vaccines typically include a modified mRNA encoding at least one immunogen, such as a viral envelope protein, cell surface binding protein, or a biologically effective/significant fragment thereof. The mRNA can be encapsulated into lipid nanoparticles or other carriers and formulated as pharmaceutical compositions that can be used to generate an immune response to pathogens, including monkeypox virus, in a subject.

### CROSS-REFERENCE TO RELATED APPLICATION

The present application claims priority under 35 U.S.C. § 119 (e) to U.S. Provisional Patent Application No. 63/428,325, filed Nov. 28, 2022, which is hereby incorporated by reference in its entirety herein.

### STATEMENT REGARDING FEDERALLY SPONSORED RESEARCH

This invention was made with Government support under W81XWH-21-1-0019 awarded by Department of Defense. The Government has certain rights in the invention.

### BACKGROUND

As of late 2022, the accumulated global case number of monkeypox infections has surpassed 47,000, including over 17,000 in US alone (CDC/WHO statistics). Because of the fast spread of monkeypox virus (MPXV), the World Health Organization has declared the ongoing monkeypox outbreak a public health emergency of international concern on Jul. 23, 2022.

Vaccination is the ultimate approach to prevent infectious disease outbreak from developing into a global pandemic. The soaring global monkeypox cases has led to a surge in demand for monkeypox vaccines, which far exceeds the currently available supply. The replication-deficient modified vaccinia Ankara (MVA) vaccine, JYNNEOS™ (commercial name in U.S., IMVANEX in Europe and IMVAMUNE in Canada) is the approved and preferred vaccine for monkeypox due to less side effects and contraindications compared to 25 ACAM2000, which is a replication-competent, live vaccinia vaccine also available for use against monkeypox. In particular, the supply of JYNNEOS™ continues to be in short supply compared to the live vaccines. However, the ACAM2000 vaccination is associated with unexpectedly high rate of myocarditis and pericarditis, and can cause severe illness in immunocompromised patients. mRNA as vaccination strategy has achieved great success in prevention of coronavirus disease and holds great promise against diverse pathogens.

Thus there is an urgent need for safe and effective vaccines against MPXV infection that can be manufactured at a global scale. The invention of the current disclosure addresses these needs.

### SUMMARY OF THE INVENTION

As described herein, the present disclosure includes a modular vaccine platform. Also provided are monkeypox (Mpox) vaccines that protect against pathogenic monkeypox species, as well as their variants. The modular platform vaccines typically include a modified mRNA encoding at least one immunogen, such as a viral envelope protein, cell surface binding protein, or a biologically effective/significant fragment thereof. In certain embodiments, the immunogen is derived from monkeypox. In certain embodiments, the mRNA is encapsulated into lipid nanoparticles or other carriers and formulated as pharmaceutical compositions that can be used to generate an immune response to pathogens, including monkeypox virus, in a subject.

As such, in one aspect, the current disclosure provides an isolated messenger ribonucleic acid (mRNA) comprising a 5′ untranslated region (UTR), a 3′ UTR, and an open reading frame encoding at least one monkeypox virus antigen, wherein the protein sequence of the at least one monkeypox virus antigen comprises all or a portion of the protein sequence of a monkeypox envelope or cell surface binding protein.

In certain embodiments, the at least one monkeypox virus antigen is selected from the group consisting of A29, E8L, M1R, A35R, B6R and any combination thereof.

In certain embodiments, the amino acid sequences encoding two or more monkeypox antigens are linked by independently selected linker sequences.

In certain embodiments, the linker sequences comprise a 2A peptide sequence.

In certain embodiments, the mRNA of the above aspects or embodiments or any aspect or embodiment disclosed herein further comprises one or more transmembrane domain and cytosolic segments (TM/Cs).

In certain embodiments, the TM/C segment is selected from the group consisting of a HLA-CAAX, a Spike, an Env, a CAR, a CD86-TLR9, an E8L, an HLA, an HLA-EPM-EABR, an HLA-EABR-CAAX, a KEL, a FIBCD1, an ANPEP, an ASGR1, a CD38, a CD40LG, a CLEC2A, a CORIN, and a GLDN TM/C domain.

In certain embodiments, the TM/C segment comprises an amino acid sequence selected from the group consisting of SEQ ID NOs: 11-19 and 28-36.

In certain embodiments, the TM/C segment comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 11-19 and 28-36.

In certain embodiments, the mRNA of the above aspects or embodiments or any aspect or embodiment disclosed herein further comprises a signal peptide.

In certain embodiments, the signal peptide is selected from the group consisting of a CD8, a SARS COV2, a tPA, an IL2, an albumin, an Env, a Secrecon, and a IgKvIII signal peptide.

In certain embodiments, the signal peptide is a CD8 signal peptide.

In certain embodiments, the signal peptide comprises an amino acid sequence selected from the group consisting of SEQ ID NOs: 20-29.

In certain embodiments, the signal peptide comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 20-29.

In certain embodiments, the isolated mRNA further comprises a reporter gene.

In certain embodiments, the reporter gene is GFP or a variant thereof.

In certain embodiments, the at least one antigen protein sequence comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 1-10.

In certain embodiments, the at least one antigen protein sequence comprises an amino acid sequence set forth in SEQ ID NOs: 1-10.

In certain embodiments, the mRNA encodes an amino acid sequence set forth in SEQ ID NO: 37.

In certain embodiments, the mRNA encodes an amino acid sequence having 75%, 80%, 85%, 90%, 91%, 92%, 93%, 94%, 95%, 96%, 97%, 98%, 99%, 99.5% or more sequence identity to SEQ ID NO: 37.

In another aspect, the current disclosure provides an isolated polynucleotide encoding the mRNA of any one of claims 1-11.

In certain embodiments, the polynucleotide comprises one or more promoters and/or a polyadenylation signal operably linked to a sequence encoding the mRNA.

In another aspect, the current disclosure provides a vector comprising the polynucleotide of any of the above aspects or embodiments or any aspect or embodiment disclosed herein.

In another aspect, the current disclosure provides a method of producing a recombinant monkeypox antigen protein, the method comprising introducing the polynucleotide and/or vector of any aspect or embodiment disclosed herein into a host cell and incubating the host cell under conditions sufficient for expression of the polynucleotide encoding the mRNA of any one of the aspects or embodiments disclosed herein, thereby producing the monkeypox antigen protein.

In another aspect, the current disclosure provides a lipid nanoparticle comprising the mRNA of any one of the above aspects or embodiments or any aspect or embodiment disclosed herein.

In certain embodiments, the molar ratio of lipid to mRNA is in the range of about 5:1 to 20:1, preferably 6:1.

In certain embodiments, the lipid nanoparticle comprises at least one ionizable cationic lipid, at least one helper lipid, at least one sterol, and/or at least one PEG-modified lipid.

In certain embodiments, the at least one ionizable cationic lipid comprises 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), PNI ionizable lipid, SM-102, DLin-MC3-DMA, DLin-KC2-DMA, ALC-0315, or a combination thereof.

In certain embodiments, the at least one helper lipid comprises 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC), 1,2-dioleoyl-sn-glycero-3-phosphocholine (DOPC), or a combination thereof.

In certain embodiments, the at least one PEG-modified lipid comprises 1,2-dimyristoyl-racglycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, mPEG-DPPE, or a combination thereof.

In certain embodiments, the at least one sterol is cholesterol.

In certain embodiments, the lipid nanoparticle comprises about 20-60% ionizable cationic lipid, about 5-25% helper lipid, about 25-55% sterol, and about 0.5-15% PEG-modified lipid.

In another aspect, the current disclosure provides a pharmaceutical composition comprising the lipid nanoparticle of any one of the aspects or embodiments disclosed herein and a pharmaceutically acceptable carrier or excipient.

In another aspect, the current disclosure provides a vaccine comprising one or more lipid nanoparticles of any one of the aspects or embodiments disclosed herein and/or the pharmaceutical composition of any of the aspects or embodiments disclosed herein, and further comprising a pharmaceutically acceptable adjuvant.

In another aspect, the current disclosure provides a method of inducing in a subject an immune response to a monkeypox virus, comprising administering to the subject the vaccine of any of the aspects or embodiments disclosed herein in an amount effective to generate the immune response.

In certain embodiments, the immune response comprises a T cell response and/or a B cell response.

In certain embodiments, the immune response comprises a neutralizing antibody response specific to the at least one monkeypox antigen protein.

In certain embodiments, the immune response inhibits infection by the monkeypox virus and/or replication of the monkeypox virus in the subject.

In certain embodiments, the subject is administered a single dose of the vaccine.

In certain embodiments, the subject is administered two or more doses of the vaccine, optionally wherein the two or more doses are administered 14-28 days apart.

In certain embodiments, each administration of the vaccine comprises a dose of mRNA of about 1 μg, 3 μg, 10 μg, 25 μg, 30 μg or 100 μg.

In certain embodiments, the effective amount is a total dose of about 1-500 μg, inclusive.

In certain embodiments, the vaccine is administered by intradermal injection, intramuscular injection, oral administration, intranasal administration, or intratracheal administration.

In certain embodiments, the subject has been exposed to, is infected with, or is at risk of infection by the monkeypox virus.

In certain embodiments, the subject is human.

### BRIEF DESCRIPTION OF THE DRAWINGS

The accompanying drawings, which are incorporated in and constitute a part of this specification, illustrate several embodiments of the disclosed method and compositions and together with the description, explain certain embodiments of the disclosure.

FIGS. 1A-IF illustrate a monkeypox mRNA vaccine design and characterization of its immunogenicity in mice. FIG. 1A, five neutralizing targets of monkeypox mRNA vaccine candidate are connected by 2A linkers and translated from the same mRNA transcript. Individual monkeypox antigen domain arrangement is shown along with antigen sequence difference between monkeypox and modified vaccinia Ankara (MVA, attenuated monkeypox vaccine progenitor). The antigen design of a multivalent monkeypox LNP-mRNA vaccine candidate, MPXVac-097, is shown in the bottom panel. FIG. 1B, the antigen sequence differences between monkeypox and MVA are displayed on corresponding homology structural models. The reported neutralizing antibody epitopes on antigen surface are colored in darker grey and monkeypox specific surface that differs from MVA is colored in lighter grey. FIG. 1C, Co-expression of five monkeypox antigens and GFP in 293T cells. The GFP positive cells with expression of five monkeypox antigens are identified by flow cytometry and fluorescence microscope. FIG. 1D, Size distribution of MPXVac-097 LNP-mRNA as determined by dynamic light scattering. FIG. 1E, Three doses of MPXVac-097 LNP-mRNA significantly increased antibody titers against A35R and E8L (n=5). Mice vaccination and blood collection schedule are illustrated on the time axis. Statistical comparisons were made between adjacent time points. FIG. 1F, Cell surface antigen recognized by mice plasma antibody elicited by MPXVac-097 LNP-mRNA×2. Monkeypox antigens expressed on 293T cell surface were recognized by plasma antibody produced from mice on day 20. SARS-COV-2 spike expressing cells served as a negative control. Data on bar plots are shown as mean±s.e.m. with individual data points in plots.

FIGS. 2A-2D illustrate TCR gene repertoire analyses in pre-vaccinated and MPXV post-boosted mice. FIG. 2A, Circos plots of the top 20 TRA and TRB V-J gene combinations for pre-vaccination (D0) and post-boost (D20) of two-dose MPXVac-097 vaccinated mice. replicate-pooled count data are presented in each plot. FIG. 2B, Bar plots for the number of clones and unique clonotypes detected between pre-vaccination (D0) and MPXV post-boost (D20) mice. FIG. 2C, Bar plots of the estimated sample diversity in TRA and TRB repertoires between pre-vaccination (D0) and MPXV post-boost (D20) mice. Estimates were calculated using the “true diversity” method, based on amino acid sequences. FIG. 2D, Bar plots of the relative distribution of clones between different ranges of top clonotypes. The data are presented with the index range of the most abundant clonotypes on the x axis and the percent of total repertoire on the y axis. All analyses were performed with n=4 paired pre-vaccination and post-boost samples. Data on bar plots are shown as mean±s.e.m. with individual data points in plots.

FIG. 3 illustrates a sequence alignment of five antigen homologs from monkeypox (GenBank ON563414.3) and MVA (GenBank AY603355.1). Transmembrane domains and ectodomains of each antigen are indicated on the sequences.

FIGS. 4A-4D illustrate a calculated electrostatic map on homolog structures of five monkeypox antigens (FIGS. 4A-4D). The surface electrostatic map was generated using CHARMM-GUI server and SWISS homology models.

FIGS. 5A-5B illustrate untransfected 293T cells as negative controls in the flow cytometry and fluorescence microscope experiments. FIG. 5A, gating strategy used in flow cytometry. FIG. 5B, five monkeypox antigens in tandem with GFP.

FIG. 6 illustrates ELISA titration curves showing binding response (OD450 on y axis) over serial dilution points of plasma samples (log₁₀ transformed dilution factor on x axis) collected from mice vaccinated with zero, one, two or three doses on MPXV LNP-mRNA on day 0, 14, 20, 28 and 42.

FIGS. 7A-7B illustrate the gating strategy used in flow cytometry to identify mice antibody bound 293T cells expressing M1R (FIG. 7A) or A35R (FIG. 7B) antigens. Plasma samples were collected on day 20 from mice immunized with two doses of MPXV LNP-mRNAs and the primary mice antibody bound to the cells was recognized by anti-mouse IgG second antibody with PE fluorophore.

FIGS. 8A-8C illustrate quality control and sample filtering of TCR sequencing in pre-vaccinated and MPXV post-boosted mice. FIG. 8A, Plots comparing the first eight components from the principal components analysis (PCA) of TCR repertoire overlap between samples. TCR repertoire overlap (amino acid sequence with variable region gene) was assessed by Morisita index score for TRA and TRB separately, and PCA was performed with pooled index scores. Plots in the upper-right triangle compare PCs, while the panels of the bottom-left triangle show Pearson correlation rho for the corresponding upper-right panel. Correlation rho are provided for total, pre-vaccination (D0), and post-boost (D20) in black, light grey, and dark grey, respectively. FIG. 8B, Scree plot of the variance explained by each PC from the PCA of TCR repertoire overlap between samples. First two PCs were chosen for further analysis based on the elbow plot method. FIG. 8C, Bar plot of the sample differences, using Malahanobis distance calculated from the first two PCs. Statistical analyses were performed by chi-squared test for each sample, and results were FDR-corrected for multiple testing. Statistical significance labels: * p<0.05; ** p<0.01; *** p<0.001; **** p<0.0001. Non-significant comparisons are not shown.

FIGS. 9A-9D illustrate a summary of statistics of TCR sequencing in pre-vaccinated and MPXV post-boosted mice. FIG. 9A, Histograms of the CDR3 amino acid lengths for TRA and TRB clonotypes between pre-vaccination (D0) and post-boost (D20) two-dose MPXVac-097 vaccinated mice. FIGS. 9B-9C, Circos, and bar plots (FIG. 9B, and FIG. 9C, respectively) of the TRA and TRB repertoire overlap between pre-vaccination (D0) and MPXV post-boost (D20) mice. Repertoire comparisons were performed using the Morisita method. FIG. 9D, Stacked bar plots of the relative abundances of clones between different ranges of clonotype frequencies. The data are presented with the index range of the most abundant clonotypes on the x axis and the percent of total repertoire on the y axis.

FIGS. 10A-10D illustrate the enhancing of mRNA antigen immunogenicity by a modular vaccine platform. FIG. 10A, Schematics illustrating a modular vaccine platform for rapid assembly and optimization of mRNA antigens. Two distinct approaches mediated by type I and II TM/Cs are used to display cytosolic antigens, such as A29L on cell surface for B cell recognition. FIG. 10B, Direct comparison of antibody response elicited by SARS-COV-2 spike and MPXVac-097 mRNA antigens alone or in combination (n=4 on day 28). FIG. 10C, tPA signal peptide improved antibody response to E8L and A29L, but not A35R, M1R or B6R (n=4). FIG. 10D, Effect of UTR pairs on the antibody response to A35R mRNA antigens (n=3).

FIGS. 11A-11D illustrate that surface display and immunogenicity of M1R were improved by modular testing of signal peptides (SP), transmembrane domain and cytosolic segments (TM/C). FIG. 11A, Various signal peptides (SP) increased cell-surface expression of M1R ectodomain (M1Re) fused to HLA TM/C. FIG. 11B, Different type I TM/C improved cell-surface expression of [CD8a SP]-M1Re. FIG. 11C, Heterologous TM/Cs fused to [CD8a SP]-M1Re significantly improved antibody response to M1R (n=5). FIG. 11D, Modulation of M1R-specific T cell response by heterologous TM/Cs.

FIGS. 12A-12G illustrate the ranking of the CST strength of SP and TM/C using multiple MPXV antigens. FIG. 12A, schematics that show the calculation of universal CST score using multiple antigen datasets. The integrated CST score is the average of normalized antigen expression (denoted as normalized CST score) of multiple antigen datasets. The normalized CST scores evaluate CST modules' strength to promote antigen surface translocation and are the antigen surface expression levels normalized by the maximum value within the experimental groups. FIGS. 12B & 12F, the integrated CST scores (FIG. 12B) and normalized CST scores (FIG. 12F) of type I signal peptides calculated from A29L, M1R and E8L three datasets. FIGS. 12C & 12G, the integrated CST scores (FIG. 12C) and normalized CST scores (FIG. 12G) of type I TM/Cs calculated from A29L and M1R antigen datasets. Statistical significance is derived from comparison of experimental groups with controls including no SP or TM/C control and native M1R TM/C control. FIG. 12D, A subset of type II TM/Cs led to higher A35R ectodomain surface expression than native A35R. FIG. 12E, Quantification of A29L surface expression mediated by type I or type II TM/Cs. The untagged antigen was stained using anti-A35R or anti-A29L antibody (n=3).

### DETAILED DESCRIPTION

Human monkeypox is a zoonotic Orthopoxvirus, and is related to smallpox, vaccinia, and variola viruses. As of late 2022, monkeypox virus (MPXV) is the most notable Orthopoxvirus affecting human populations, and represents the first wide-spread epidemic of Orthopoxvirus infections since the eradication of smallpox by the World Health Organization (WHO), which was completed in 1980. Because of its fast spread, the WHO declared the monkeypox outbreak a global public-health emergency on Jul. 23, 2022. A retrospective study recently reported evidence of asymptomatic or undiagnosed monkeypox cases during early stage of the outbreak suggesting that asymptomatic cases have and continue to fuel this epidemic, and that symptom-based testing and quarantining may not be sufficient to provide containment. In addition to testing and quarantining, a key strategy to contain MPXV transmission is the use of specific vaccines for high-risk groups and those in close contact with infected patients. As the monkeypox outbreak grows rapidly, the demand for monkeypox vaccines has surged drastically and many countries are facing the vaccine supply shortages.

Vaccination is the ultimate approach to prevent infectious disease outbreak from developing into a global pandemic. Replication-deficient modified vaccinia ankara (MVA) vaccine, JYNNEOS™ (commercial name in U.S., IMVANEX in Europe and IMVAMUNE in Canada) is the approved and preferred vaccine for monkeypox due to less side effects and contraindications compared to ACAM2000, which is a replication-competent vaccinia vaccine also available for use against monkeypox. The monkeypox vaccine in short supply is JYNNEOS, while ACAM2000 supply is abundant. However, the ACAM2000 vaccination is associated with unexpectedly high rate of myocarditis and pericarditis, and can cause severe illness in immunocompromised patients. The authorization of JYNNEOS and ACAM2000 is based on 1) a phase 3 trial assessing neutralizing antibody response to vaccinia virus in vaccinees of MVA and ACAM2000; and 2) a MPXV challenge experiment in macaques showing 100% protection after two doses of MVA or one dose of ACAM2000 injection. Although no data is currently available on the efficacy of either vaccine against 2022 MPXV in human, a series of clinical trials and cohort studies are underway to evaluate MVA vaccine's efficacy, safety and protection durability.

Unlike traditional vaccines directly delivered to extracellular spaces, an mRNA vaccine is expressed inside recipient's cells and relies on additional mechanisms to translocate to the extracellular space, where it will be recognized by immune cells. The safety and efficacy of mRNA vaccine technology has been most clearly demonstrated by the tremendous success of COVID mRNA vaccine, which has proven to be safe and effective across the globe in both initial clinical trials and real-world data. It is a versatile platform that can be adopted to develop mRNA vaccines against other infectious diseases, including monkeypox. Although MVA-based vaccine has less side effect than ACAM2000, its >=3 grade adverse event rate is 7.7% in recipients. In contrast, the >=3 grade adverse event rate of COVID mRNA vaccine is similar to placebo group (1.5% in vaccine group vs. 1.3% in placebo group). The MVA vaccine contains around 200 proteins, many of which are not immunogenic and likely associated with undesired side effects. Removal of undesired components in MPXV vaccines would improve its safety profile, which is the case for MVA that lost large fragments of genome during attenuation. Simplification of MPXV vaccine could facilitate rapid vaccine optimization. In addition, the manufacturing of mRNA vaccine can be achieved in vitro independent of complex cell culture as opposed to inactivated or attenuated vaccine; and is easily scalable by biochemical reactions. These features together make mRNA vaccine promising to enable the world to quickly fill the gap between vaccine supply and demand during a disease outbreak. The shortage of monkeypox vaccine and adaptability of mRNA vaccine prompt the idea of developing MPXV-targeting mRNA vaccine. However, whether the mRNA-based MPXV antigens can elicit significant immune response in vivo remains a critical question to be answered.

The mechanisms of antigen protein expression inside cells and the need for mRNA translocation pose potential challenges for the development of certain mRNA vaccines. Because of this isolated expression inside cells, certain mRNA antigens may elicit less potent immune response than traditional antigens. A29L protein immunization in vivo has been reported to elicit significant neutralizing antibody response. However, more recent data has shown that vaccination with A29L lipid nanoparticle (LNP) mRNA results in poor antibody response. Further analysis of A29L sequence and cellular localization revealed that its sequence lacks a membrane translocation signal, and it remains in the cytosol when expressed alone. The lack of a reliable way to translocate mRNA antigens to extracellular space is one of critical factors that explain why certain mRNA antigens elicit less potent immune responses than traditional antigens.

Many viral glycoproteins exist in a network of protein complexes and single antigen may not possess cell surface translocation (CST) signal as potent as SARS-COV-2 spike of which surface translocation solely relies on its own. How to improve these mRNA antigens' CST becomes a critical question to address. Because of the correlation between mRNA antigen CST and immunogenicity, immune responses to mRNA antigen can be improved by modifying its cell surface translocation (CST) signal using a modular vaccine platform. Disclosed herein is a library of CST sequences from single-pass transmembrane proteins on plasma membrane, which establishes a modular vaccine platform. Also disclosed herein is the recombination of antigen ectodomain with these CST sequences, and the subsequent characterization of the chimeric antigens' CST by flow surface staining, as well as their LNP mRNA's immunogenicity in vivo. Thus the modular vaccine platform (MVP) of the current disclosure consists of parental vectors with different UTR pairs, type II and type I CST module plasmids. The identified CST sequences were grouped based on membrane topology (Type I and II) and separated into modules which include signal peptide (SP), extracellular hinge+transmembrane domain TM and cytosolic segments (C). The parental vector contains 5′ & 3′ UTR pairs, GFP and type IIS digestion sites to enable dropout selection and rapid modular assembly of chimeric antigens.

The invention of the current disclosure also demonstrates the development of potent polyvalent MPXV vaccines to protect against pathogenic MPXV infections. LNP-mRNA vaccines were generated with mRNAs specifically encoding immuno-relevant MPXV antigen proteins. In certain embodiments, these antigen proteins are MPXV envelope proteins, cell surface binding proteins, or polypeptides derived from MPXV envelope or cell surface binding proteins. Thus, the vaccines described herein can potently neutralize MPXV infections. Also described are vaccines in which AAVs and virus like particles (VLPs) are used as the carriers of MPXV mRNAs and/or proteins encoded therefrom.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Definitions

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Recitation of ranges of values are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +10%; in other forms the values may range in value either above or below the stated value in a range of approximately #5%; in other forms the values may range in value either above or below the stated value in a range of approximately #2%; in other forms the values may range in value either above or below the stated value in a range of approximately +1%. The preceding ranges are intended to be made clear by context, and no further limitation is implied.

“Chimeric” as used in the context of a nucleic acids and proteins describes a non-naturally occurring polynucleotide or polypeptide that is or has a sequence that is made by an artificial combination of two or more otherwise separated segments of sequence. In certain non-limiting embodiments, the sequences combined to form the chimeric nucleic acid or protein are derived from two or more different viral species or strains. This artificial combination is often accomplished by chemical synthesis or by the artificial manipulation of isolated segments of nucleic acids, e.g., by genetic engineering techniques known in the art (e.g., to facilitate addition, substitution, or deletion of a portion of the nucleic acid).

The term “effective amount” means a quantity sufficient to provide a desired pharmacologic and/or physiologic effect.

As used herein, the term “encapsulate” means to enclose, surround or encase.

“Heterologous” is used herein in the context of two more elements having a different, non-native relation, relative position, or structure. The elements can include, but are not limited to, naturally occurring elements from the same or different organisms, chimeric elements, synthetic or engineered elements, etc., provided that the elements are not found in nature in the same relation, relative position, or structure.

“Introduce,” as used herein, refers to bringing into contact. By “contact” or “contacting” is meant to allow or promote a state of immediate proximity or association between at least two elements. For example, to introduce a composition (e.g., a vector containing a sequence encoding a monkeypox virus antigen protein or fragment thereof) to a cell is to provide contact between the cell and the composition. The term encompasses penetration of the contacted composition to the interior of the cell by any suitable means, e.g., via transfection, electroporation, transduction, gene gun, nanoparticle delivery, etc.

“Isolated” means altered or removed from the natural state. An isolated nucleic acid can exist in substantially purified form, or can exist in a non-native environment such as, for example, a host cell. An “isolated” nucleic acid encompasses a nucleic acid segment or fragment which has been separated from sequences which flank it in a naturally occurring state, e.g., a DNA fragment which has been removed from the sequences which are normally adjacent to the fragment in a genome in which it naturally occurs. The term also applies to nucleic acids which have been substantially purified from other components which naturally accompany the nucleic acid (e.g., RNA or DNA or proteins, which naturally accompany it in the cell). The term therefore includes, for example, a mRNA, or recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. Isolated does not require absolute purity, and can include protein, peptide, nucleic acid, or virus molecules that are at least 50% isolated, such as at least 75%, 80%, 90%, 95%, 98%, 99%, or even 99.9% isolate.

The term “mutation” refers to a change in a sequence resulting in an alteration from a given reference sequence. Mutations include a substitution of a residue within a sequence, e.g., a nucleic acid or amino acid sequence, with another residue, or a deletion or insertion of one or more residues within a sequence. In certain non-limiting embodiments, the mutation can be a deletion, insertion, duplication, rearrangement, and/or substitution of at least one deoxyribonucleic acid base such as a purine (adenine and/or guanine) and/or a pyrimidine (thymine, uracil and/or cytosine). In certain non-limiting embodiments, the mutation can be a deletion, insertion, or substitution of at least one amino acid residue in a polypeptide. In certain non-limiting embodiments, mutations are described by identifying the original residue followed by the position of the residue within the sequence and by the identity of the newly substituted residue (e.g., K986P, V987P). Mutations may or may not produce discernible changes in the observable characteristics (phenotype) of a subject.

As used herein, “open reading frame” or “ORF” refers to a sequence which does not contain a stop codon in a given reading frame.

The term “operably linked” or “operationally linked” refers to functional linkage between a regulatory sequence and a heterologous nucleic acid sequence permitting them to function in their intended manner (e.g., resulting in expression of the latter). The term encompasses positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

The % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100×the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “subject” refers to any individual, organism or entity. Typical subjects include animals (e.g., mammals such as mice, rats, rabbits, goats, pigs, chimpanzees, or horses, non-human primates, and humans) and/or plants. The term does not denote a particular age or sex. Thus, adult and newborn subjects, as well as fetuses, whether male or female, are intended to be covered. The subject may be healthy or suffering from or susceptible to a disease, disorder, or condition.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. “Expression vector” refers to a vector containing a polynucleotide having expression control sequences operatively linked to a nucleotide sequence to be expressed. An expression vector contains sufficient cis-acting elements for expression; other elements for expression can be supplied by the host cell or in an in vitro expression system. Expression vectors include all those known in the art, such as cosmids, plasmids (e.g., naked or contained in liposomes), phagemids, BACs, YACs, and viral vectors (e.g., vectors derived from lentiviruses, retroviruses, adenoviruses, and adeno-associated viruses) that incorporate the recombinant polynucleotide.

### Compositions

Nucleic acids, and compositions and methods of used thereof are disclosed. In particular, compositions, including pharmaceutical compositions, for the preparation and/or formulation of nucleic acids, and which are useful for the generation of vaccines are provided. The compositions are especially useful for delivery of nucleic acids, e.g., a ribonucleic acid (RNA) inside a cell, whether in vitro, in vivo, in situ or ex vivo.

Nucleic acids include any compound and/or substance that constitute a polymer of nucleotides, and hence, can be referred to as polynucleotides. Exemplary nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), and hybrids thereof.

mRNAs

In certain embodiments, the disclosed nucleic acids are, or include ribonucleic acids. A non-limiting ribonucleic acid is messenger RNA (mRNA). The term messenger RNA (mRNA) can refer to any ribonucleic acid which directly encodes a polypeptide of interest. Thus, the disclosed mRNAs are capable of being translated to produce one or more encoded polypeptides of interest. In certain non-limiting embodiments, the mRNAs are produced by in vitro transcription.

The mRNAs can be of any suitable length. For example, the length can vary depending upon the size of the encoded polypeptide. mRNA molecules are typically between 200 and 10,000 nucleotides in length. In certain non-limiting embodiments, a mRNA includes about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or 100,000 nucleotides, with or without the poly(A) tail, 5′ UTR, and/or 3′ UTR.

The mRNAs can be codon optimized. For example, the mRNAs can be codon optimized for expression in a eukaryotic cell. The eukaryotic cell can be those of or derived from a particular organism, such as a plant or a mammal, including but not limited to human, or non-human eukaryote or animal or mammal, e.g., mouse, rat, rabbit, dog, livestock, or non-human mammal or primate. Codon-optimization describes gene engineering approaches that use changes of rare codons to synonymous codons that are more frequently used in the cell type of interest with the aim of increasing protein production. In general, codon optimization involves modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA, which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa dot orjp/codon/and these tables can be adapted in a number of ways. See for example, Nakamura, Y., et al., Nucl. Acids Res., 28:292 (2000). Computer algorithms for codon optimizing a particular sequence for expression in a particular host cell are also available, such as Gene Forge (Aptagen; Jacobus, PA). In certain non-limiting embodiments, one or more codons (e.g., 1, 2, 3, 4, 5, 10, 15, 20, 25, 50, or more, or all codons) in a mRNA corresponds to the most frequently used codon for a particular amino acid.

Typically, the disclosed isolated messenger ribonucleic acids (mRNAs) contain a 5′ untranslated region (UTR), a 3′ UTR, and an open reading frame (also referred to as coding region). In certain non-limiting embodiments, the mRNAs further include a 5′ cap or an analog thereof, a poly(A) tail, one or more modified nucleotides, or a combination thereof. In certain embodiments, the mRNAs include at least a 5′ cap or analog thereof, a 5′ UTR, a 3′ UTR, one or more open reading frames, and a poly(A) tail. In certain embodiments, the mRNAs include at least a 5′ cap or analog thereof, a 5′ UTR, a 3′ UTR, one or more open reading frames, a poly(A) tail, and one or more modified nucleotides.

The mRNA can include different caps or cap analogs (e.g., ARCA). The body of the mRNA can use modified nucleosides. The one or more coding sequences or open reading frames can include various elements such as signal peptides, localization signals (e.g., NLSs), inteins, etc. The structures of the mRNA can be engineered to optimize GC motifs, folding, circularization signals, and/or structured UTR elements.

In certain non-limiting embodiments, the open reading frame encodes a pathogen derived antigen, such as a bacterial, fungal, or viral protein. In certain non-limiting embodiments, the open reading frame encodes all or a portion of one or more proteins from a virus, such as but not limited to a monkeypox virus. In certain embodiments, the open reading frame encodes one or more monkeypox envelope protein sequences, wherein the envelope protein sequences include all or a portion of one or more monkeypox envelope proteins.

Thus, a non-limiting mRNA includes a 5′ UTR, a 3′ UTR, and an open reading frame encoding at least one antigenic protein sequence derived from monkeypox virus, including variants thereof, and optionally a 5′ cap or an analog thereof, a poly(A) tail, one or more modified nucleotides, or a combination thereof.

In some certain embodiments, the mRNA is a chimeric (also referred to as hybrid) mRNA. The chimeric mRNA can include one or more (e.g., 1, 2, 3, 4, 5) open reading frames which encode a chimeric (hybrid) antigenic protein or subunit or other fragment thereof which has sequences from different viral species or variants. For example, a chimeric mRNA can include a 5′ UTR, a 3′ UTR, and one open reading frame which encodes two or more different antigenic protein sequences (e.g., complete envelope proteins or subunits or other fragments thereof) in frame with each other from distinct monkeypox virus species or variants thereof. As another example, a chimeric mRNA can include a 5′ UTR, a 3′ UTR, and two or more open reading frames, wherein each open reading frame encodes a different antigenic protein sequence, wherein each antigenic protein sequence includes an antigen protein or subunit or other fragment thereof from the antigenic protein of a distinct monkeypox species or variant thereof. In certain non-limiting embodiments, the chimeric mRNA includes a linker or other domain intervening between the two or more open reading frames. In certain non-limiting embodiments, the linkers comprise 2A peptides, or 2A self-cleaving peptides which result in the expression of the various antigenic protein sequences as distinct polypeptides rather than a single contiguous polypeptide.

In certain non-limiting embodiments, the mRNA includes an open reading frame. In certain non-limiting embodiments, the open reading frame encodes an antigenic monkeypox envelope protein or cell surface binding protein or subunit or other fragment thereof (e.g., A29, E8L, M1R, A35R and B6R), wherein the sequences encoding the antigenic proteins are separated by linker sequences. In certain non-limiting embodiments, the linker sequences comprise 2A peptides, or some other self-cleaving or cleavable peptide such that single open reading frame can be translated as one or more separate polypeptides. 2A peptides are 18-22 amino acid long viral oligopeptides that mediate cleavage of polypeptides during translation in eukaryotic cells. The mechanism of 2A-mediated self-cleavage is thought to be ribosome skipping the formation of a glycyl-prolyl peptide bond at the C-terminus of the 2A. Suitable 2A self-cleaving peptides include F2A (foot-and-mouth disease virus), E2A (equine rhinitis A virus), P2A (porcine teschovirus-1 2A), and T2A (thosea asigna virus 2A).

### 5′ Cap

Typically, the 5′ cap of an mRNA is involved in nuclear export, increasing mRNA stability and binding the mRNA Cap Binding Protein (CBP), which is responsible for mRNA stability in the cell and translation competency through the association of CBP with poly(A) binding protein to form the mature cyclic mRNA species. Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. In certain non-limiting embodiments, the mRNA contains a non-hydrolyzable cap, which can prevent or hinder decapping and thus increase the mRNA half-life. Because cap structure hydrolysis requires cleavage of 5′-ppp-5′ phosphodiester linkages, the 5′ cap can include modified nucleotides to prevent such hydrolysis.

The 5′ cap may be a single nucleotide or a series of nucleotides. For example, the cap may include from 1 to 10, e.g., 2-9, 3-8, 4-7, 1-5, 5-10, or at least 1 or 2, or 10 or fewer nucleotides in length. In certain non-limiting embodiments, the cap is absent.

Cap analogs differ from natural (e.g., endogenous, wild-type or physiological) 5′-caps in their chemical structure, while retaining cap function. Cap analogs may be chemically (e.g., non-enzymatically) or enzymatically synthesized and/or linked to a nucleic acid molecule. For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-O atom of the other, unmodified, guanine becomes linked to the 5′-terminal nucleotide of the capped nucleic acid molecule (e.g., mRNA). The N7- and 3′-O-methylated guanine provides the terminal moiety of the capped nucleic acid molecule. Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In certain non-limiting embodiments, a 5′ cap may include endogenous caps or cap analogs. For example, a 5′ cap may include a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Suitable 5′ caps or analogs that can be included in the mRNAs are known in the art and include, without limitation, 7mG(5′)ppp(5′)N,pN2p (cap 0), 7mG(5′)ppp(5′)NImpNp (cap 1), 7mG(5′)-ppp(5′)NlmpN2mp (cap 2), ARCA, beta-S-ARCA, m7G, mCAP, inosine, N1-methyl-guanosine, 2′-fluoro-guanosine, 7-deaza-guanosine, 8-oxo-guanosine, 2-amino-guanosine, LNA-guanosine, tri-methylgranosine (TMG), nicotinamide adenine dinucleotide (NAD), cap AG, cap AU, cap GG, and 2-azido-guanosine.

### Untranslated Regions

Untranslated regions (UTRs) are regions of a gene that are transcribed, but not translated. Generally, the 5′UTR starts at the transcription start site and continues to the start codon but does not include the start codon; whereas the 3′UTR starts immediately following the stop codon and continues until the transcriptional termination signal. 5′ UTRs can harbor specific regions, like Kozak sequences which are be involved in the initiation of translation by the ribosome. 5′ UTRs also have been known to form secondary structures which are involved in elongation factor binding. The UTRs can have important regulatory effects on an associated mRNA, for example impacting stability and/or translation of the mRNA. Generally, translational efficiency (including activation or inhibition of translation) of mRNAs can be controlled by the UTRs. In certain non-limiting embodiments, the regulatory features of a UTR can be incorporated into the disclosed mRNAs, to enhance the stability of the molecule. In certain non-limiting embodiments, the mRNAs are engineered to contain the UTRs found in abundantly expressed genes to enhance the enhance the stability and protein production from the mRNA. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of an mRNA. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

### Poly(A) Tails

During RNA processing, a long chain of adenine nucleotides, referred to as the poly(A) tail, may be added to a polynucleotide such as an mRNA in order to increase stability. Immediately after transcription, the 3′ end of the transcript may be cleaved to free a 3′ hydroxyl. Then, poly-A polymerase adds a chain of adenine nucleotides to the RNA. The process, called polyadenylation, adds a poly(A) tail that can be between, for example, approximately 100 and 250 residues long.

In certain non-limiting embodiments, the poly(A) tail includes about 10-100, about 100-300, about 100-250, or about 100-200 adenines. In certain non-limiting embodiments, the poly(A) tail contains about 10, 15, 20, 25, 30, 35, 40, 45, 50, 55, 60, 65, 70, 75, 80, 85, 90, 95, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides.

### Modified Nucleotides

The mRNA can be modified or unmodified. The mRNA can be modified for example, to optimize translation, and/or to confer increased stability and/or expression. In certain non-limiting embodiments, a mRNA or other modified polynucleotide may exhibit reduced degradation when introduced to a cell as compared to a corresponding unmodified polynucleotide.

The modified mRNA or other modified polynucleotide can incorporate a number of chemical changes to the nucleotides, including changes to the nucleobase, the ribose or deoxyribose sugar, and/or the phosphodiester linkage. One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain forms, modifications (e.g., one or more modifications) are present in each of the sugar and the internucleotide linkage.

Backbone phosphate groups can be modified by replacing one or more of the oxygen atoms with a different substituent. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates). Phosphorothioate DNA and RNA have increased nuclease resistance, and subsequently, a longer half-life in a cellular environment.

In certain embodiments, the mRNA or other polynucleotide includes one or more modified nucleotides. For example, the mRNA or other polynucleotide can include one or more modified guanine-, adenine-, cytosine-, thymidine-, and/or uridine-containing nucleotides. Suitable modified nucleotides/nucleosides include, without limitation, pseudouridine, N1-methyl-pseudouridine, N1-Methylpseudouridine-5′-Triphosphate-(N-1081), 1-ethylpseudouridine, 2-thiouridine, 4′-thiouridine, 5-methoxyuridine, 5-methoxyuridine, N6-methyladenosine, 5-methylcytosine, 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine (e.g., 5-iodo-cytidine), 5-hydroxymethyl-cytidine, 1-methyl-pseudoisocytidine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 1-methyl-adenosine, 2-methyl-adenine, N6-methyl-adenosine, 2-methylthio-N6-methyl-adenosine, N6-isopentenyl-adenosine, inosine, 1-methyl-inosine, wyosine, methylwyosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, and 6-methoxy-guanosine. For example, N1-Methylpseudouridine-5′-Triphosphate-(N-1081) can be utilized during in vitro transcription so that it is incorporated into the mRNA.

In certain non-limiting embodiments, all of the instances of a given nucleotide (e.g., every G, every A, every C, every T, or every U) are modified. In certain non-limiting embodiments, a fraction of the instances of a given nucleotide are modified. For example, about 0.1%, 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a given nucleotide can be modified.

As a non-limiting example, the nucleotide uridine may be substituted with a modified nucleotide described herein, such as N1-methyl-pseudouridine. In certain non-limiting embodiments, the uridine in the mRNA is partially substituted. For example, about 0.1%, 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the uridine in a given mRNA may be substituted with a modified nucleotide, such as N1-methyl-pseudouridine. For example, in some certain embodiments, about 50% of uridine is substituted with a modified nucleotide, such as N1-methyl-pseudouridine.

### Open Reading Frame Encoded Polypeptides

The mRNAs contain sequences that encode polypeptides of interest. For example, an mRNA can contain one or more open reading frames, each of which encodes one or more polypeptides. Typically, the open reading frame encodes an antigen (e.g., protein or peptide) from a pathogenic microorganism, such as bacteria, fungi, protozoa, or virus. In certain non-limiting embodiments, the open reading frame encodes one or more proteins from a virus, or an immune-response inducing fragment or variant thereof.

### Monkeypox Polypeptides

In certain non-limiting embodiments, the mRNA includes an open reading frame that encodes one or more immunogenic proteins or subunits or other fragments thereof from a monkeypox virus. Monkeypox viruses, like other Orthopoxviruses are large, brick-shaped, enveloped viruses which possess a linear double-stranded DNA genome around 170-250 kilobases in length.

Orthopoxviruses are a genus of the Poxviridae family and Chordopoxvirinae sub-family. The most historically notable member of the genus is the Variola virus, which causes smallpox disease in humans.

Unlike other DNA viruses, poxviruses replicate in the cytoplasm of infected cells, building special structures called virus factories or Guarnieri inclusion bodies. The Orthopoxvirus encodes 150-200 different genes, including a number of distinct envelope and membrane surface proteins. In certain non-limiting embodiments, the mRNA open reading frame encodes one or more MPXV envelope or cell surface or membrane surface proteins, or an immune response-inducing subunit, fragment, or variant derived therefrom. Representative examples of antigenic envelope or cell/membrane surface proteins include, but are not limited to A29, E8L, M1R, A35R and B6R. It is anticipated that any MPXV protein which is capable of inducing a productive immune response in the host would be acceptable for use in the mRNAs of the invention.

Exemplary gene, protein, and genomic sequences of the foregoing MPXV species and strains are known in the art.

```
An exemplary amino acid sequence of a MPXV E8L protein is:
  (SEQ ID NO: 1)
MPQQLSPINIETKKAISDARLKTLDIHYNESKPTTIQNTGKLVRINFKGGYISGGFLPNEYVLSTHIH
IYWGKEDDYGSNHLIDVYKYSGEINLVHWNKKKYSSYEEAKKHDDGIIIIAIFLQVSDHKNVYFQKIV
NQLDSIRSANMSAPFDSVFYLDNLLPSTLDYFTYLGTTINHSADAAWIIFPTPINIHSDQLSKERTLL
SSSNHEGKPHYITENYRNPYKLNDDTQVYYSGEIIRAATTSPVRENYFMKWLSDLREACFSYYQKYIE
GNKTFAIIAIVFVFILTAILFLMSQRYSREKQN.
An exemplary amino acid sequence of a MPXV MIR protein is:
  (SEQ ID NO: 3)
MGAAASIQTTVNTLSERISSKLEQEANASAQTKCDIEIGNFYIRQNHGCNITVKNMCSADADAQLDAV
LSAATETYSGLTPEQKAYVPAMFTAALNIQTSVNTVVRDFENYVKQTCNSSAVVDNKLKIQNVIIDEC
YGAPGSPTNLEFINTGSSKGNCAIKALMQLTTKATTQIAPRQVAGTGVQFYMIVIGVIILAALFMYYA
KRMLFTSTNDKIKLILANKENVHWTTYMDTFFRTSPMIIATTDIQN.
An exemplary amino acid sequence of a MPXV B6R protein is:
  (SEQ ID NO: 5)
MKTISVVTLLCVLPAVVYSTCTVPTMNNAKLTSTETSENDKQKVTFTCDSGYHSLDPNAVCETDKWKY
ENPCKKMCTVSDYVSELYDKPLYEVNSTMTLSCNGETKYFRCEEKNGNTSWNDTVTCPNAECQPLQLE
HGSCQPVKEKYSFGEYMTINCDVGYEVIGVSYISCTANSWNVIPSCQQKCDIPSLSNGLISGSTFSIG
GVIHLSCKSGFTLTGSPSSTCIDGKWNPILPTCVRSNEEFDPVDDGPDDETDLSKLSKDVVQYEQEIE
SLEATYHIIIMALTIMGVIFLISIIVLVCSCDKNNDQYKFHKLLP.
An exemplary amino acid sequence of a MPXV A35R protein is:
  (SEQ ID NO: 7)
MMTPENDEEQTSVFSATVYGDKIQGKNKRKRVIGLCIRISMVISLLSMITMSAFLIVRLNQCMSANKA
AITDSAVAVAAASSTHRKVVSSTTQYDHKESCNGLYYQGSCYILHSDYKSFEDAKANCAAESSTLPNK
SDVLTTWLIDYVEDTWGSDGNPITKTTSDYQDSDVSQEVRKYFCT.
An exemplary amino acid sequence of a MPXV A29L protein is:
  (SEQ ID NO: 9)
MDGTLFPGDDDLAIPATEFFSTKAAKNPETKREAIVKAYGDDNEETLKQRLTNLEKKITNITTKFEQI
EKCCKRNDEVLFRLENHAETLRAAMISLAKKIDVQTGRHPYE.
```

Also disclosed are variants of any of the encoded proteins or peptides described herein (e.g., a envelope or cell/membrane surface protein or subunit or other fragment thereof). For example, the mRNA can include an open reading frame that encodes a variant of any of the disclosed membrane or cell/membrane surface proteins or subunits or other fragments thereof. In certain non-limiting embodiments, suitable encoded polypeptides include variants of any one of SEQ ID NOs: 1-10 having, for example, at least 70%, 75%, 80%, 85%, 90%, 95%, 96%, 97%, 98%, 99%, or more sequence identity to any one of SEQ ID NOs: 1-10.

Suitable variants can include at least one point mutation or substitution (e.g., 1, 2, 3, 4, 5 or more mutations) at any amino acid residue relative to a reference (e.g., SEQ ID NOs: 1-10, such as but not limited to SEQ ID NOs: 1-10). Amino acid substitutions in certain non-limiting embodiments include conservative amino acid substitutions, although non-conservative substitutions can also be used. Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

### Other Polypeptides

Also provided are isolated nucleic acid molecules or polynucleotides that comprise or encode one or more transmembrane domain and cytosolic segments (TM/Cs). TM/Cs, which can comprise type I and II transmembrane domain and cytosolic segments (TM/Cs) are specialized polypeptide sequences which are used to display cytosolic antigens, e.g. A29L, on cell surface for B cell recognition. In certain embodiments, the TM/C can comprise any TM/C sequence which would be useful in the display of antigens on the surface of cells comprising the polynucleotides and/or polypeptides of the current disclosure. Examples of TM/C segment sequences that can be used in the polynucleotides and polypeptides of the invention of the current disclosure include, but are not limited to HLA-CAAX TM/C, Spike TM/C, Env TM/C, CAR TM/C, CD86-TLR9 TM/C, E8L TM/C, HLA TM/C, HLA-EPM-EABR TM/C, HLA-EABR-CAAX TM/C, KEL TM/C, FIBCD1 TM/C, ANPEP TM/C, ASGR1 TM/C, CD38 TM/C, CD40LG TM/C, CLEC2A TM/C, CORIN TM/C, and GLDN TM/C domains among others, including variants and derivatives thereof. In certain embodiments, the TM/C domain or sequence comprises an amino acid sequence selected from the group consisting of SEQ ID NOs: 11-19 and 28-36. In certain embodiments, the TM/C domain or sequence comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 11-19 and 28-36.

Also provided are isolated nucleic acid molecules or polypeptides encoding or comprising a signal peptide sequence or domain. Signal peptide sequences or domains mediate the intracellular trafficking of peptides translated in the cytosol or endoplasmic reticulum, which directs the translated peptide into the secretory pathway and eventually to the cell surface. Examples of signal peptides that can be used in the isolated nucleic acids or polypeptides of the invention of the present disclosure include, but are not limited to, CD8, SARS-COV2, tPA, IL2, albumin, Env, Secrecon, and a IgKvIII signal peptides and variants and derivatives thereof. In certain embodiments, the signal peptide is a CD8 signal peptide. In certain embodiments, the signal peptide sequence comprises an amino acid sequence selected from the group consisting of SEQ ID NOs: 20-29. In certain embodiments, the signal peptide sequence comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 20-27.

### Other Polynucleotides

Also provided are isolated nucleic acid molecules or polynucleotides that encode the disclosed mRNAs. In certain non-limiting embodiments, the nucleic acid molecule/polynucleotide is or includes DNA. The polynucleotide can include one or more promoters and/or a polyadenylation signal operably linked to a sequence encoding the mRNA. In certain non-limiting embodiments, the polynucleotide is, or is contained within, a plasmid. In certain non-limiting embodiments, the polynucleotide is, or is contained within, a vector, such as an expression vector.

Expression vectors include all those known in the art, such as cosmids, plasmids (e.g., naked or contained in liposomes), phagemids, artificial chromosomes (e.g., BACs, YACs), and viral vectors (e.g., vectors derived from lentiviruses, retroviruses, adenoviruses, and adeno-associated viruses) that incorporate the polynucleotide.

In certain non-limiting embodiments, a polynucleotide (e.g., the portion thereof encoding a mRNA) is operably linked to a control element, e.g., a transcriptional control element, such as a promoter. The transcriptional control element may be functional in either a eukaryotic cell, e.g., a mammalian cell, or a prokaryotic cell (e.g., bacterial or archaeal cell). In certain non-limiting embodiments, a polynucleotide (e.g., the portion thereof encoding a mRNA thereof) is operably linked to multiple control elements that allow expression of the polynucleotide sequence encoding a mRNA in either prokaryotic or eukaryotic cells. Depending on the host/vector system utilized, any of a number of suitable transcription and translation control elements, including constitutive and inducible promoters, transcription enhancer elements, transcription terminators, etc. may be used in the expression vector (e.g., U6 promoter, HI promoter, CMV promoter, T7 promoter, SV40 promoter, bGH poly(A) signal, SV40 poly(A) signal, etc.).

Numerous vectors and expression systems are commercially available from commercial vendors including Addgene, Novagen (Madison, WI), Clontech (Palo Alto, CA), Stratagene (La Jolla, CA), and Invitrogen/Life Technologies (Carlsbad, CA). Suitable expression vectors include, but are not limited to, viral vectors such as viral vectors based on vaccinia virus, poliovirus, adenovirus, adeno-associated virus, SV40, herpes simplex virus, human immunodeficiency virus, retroviral vectors (e.g., Murine Leukemia Virus, spleen necrosis virus, and vectors derived from retroviruses such as Rous Sarcoma Virus, Harvey Sarcoma Virus, avian leukosis virus, lentivirus, human immunodeficiency virus, myeloproliferative sarcoma virus, and mammary tumor virus), and the like. The viral vector can be derived from a DNA virus (e.g., dsDNA or ssDNA virus) or an RNA virus (e.g., a ssRNA virus).

Numerous suitable expression vectors are known to those of skill in the art, and many are commercially available, including, pXTI, pSG5 (Stratagene), pSVK3, pBPV, pMSG, pCDNA 3.1, and pSVLSV40 (Pharmacia). However, any other vector may be used so long as it is compatible with the host cell.

Any cell may be used in accordance with the foregoing. In certain non-limiting embodiments, the cell is a prokaryotic cell (e.g., an archaeal or bacterial cell). In certain non-limiting embodiments, the cell is E. coli. In other forms, the cell is a eukaryotic cell. For example, the cell can be a cell of a single-cell eukaryotic organism, a plant cell, an algal cell, a fungal cell (e.g., a yeast cell). The cell can be a mammalian cell. The mammalian cell can be human or non-human mammal, e.g., primate, bovine, ovine, porcine, canine, rodent, monkey, rat, or mouse cell.

Generation of the polynucleotides can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

### Delivery Vehicles

Also provided are vehicles for delivering or introducing the disclosed nucleic acids and compositions thereof to a cell. For example, vehicles for the introduction or production (e.g., transcription) of the disclosed mRNAs in a cell or tissue are described. Such vehicles include polynucleotides, such as plasmids and other vectors described above, which contain sequences encoding the mRNA. In certain non-limiting embodiments, viral vectors, virus-like particles, and/or lipid nanoparticles contain or encapsulate the disclosed mRNAs or polynucleotides encoding the disclosed mRNAs.

### AAV

In certain non-limiting embodiments, the vector encoding a vaccine antigen (e.g., mRNA) is a viral vector. In certain non-limiting embodiments, the viral vector is an adeno-associated virus (AAV) vector.

AAV is a non-pathogenic, single-stranded DNA virus that has been actively employed over the years for delivering therapeutic genes in both in vitro and in vivo systems (Choi, et al., Curr. Gene Ther., 5:299-310, (2005)). AAV belongs to the parvovirus family and is dependent on co-infection with other viruses, mainly adenoviruses, in order to replicate. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat is comprised of 60 proteins arranged into an icosahedral structure with the capsid proteins in a molar ratio of 1:1:10 (VP1:VP2:VP3).

Recombinant AAV vectors having no Rep and/or Cap genes can be non-integrating. In the absence of Rep proteins, ITR-flanked transgenes encoded within rAAV can form circular concatemers that persist as episomes in the nucleus of transduced cells. Because recombinant episomal DNA does not integrate into host genomes, it will eventually be diluted over time as the cell undergoes repeated rounds of replication. This will eventually result in the loss of the transgene and transgene expression.

The sequences placed between the ITRs will typically include a promoter, gene of interest (e.g., encoding a disclosed mRNA), and a terminator. The promoter can be naturally-occurring or non-naturally occurring. In many cases, strong, constitutively active promoters are desired for high-level expression of the gene of interest. Examples of promoters include, but are not limited to, viral promoters, plant promoters and mammalian promoters. Commonly used promoters include the CMV (cytomegalovirus) promoter/enhancer, EF1a (elongation factor 1a), SV40 (simian virus 40), chicken β-actin and CAG (CMV, chicken β-actin, rabbit β-globin) and variants thereof. All of these promoters provide constitutively active, high-level gene expression in most cell types. Some of these promoters are subject to silencing in certain cell types, therefore this consideration can be evaluated for each application.

Examples of terminators include, but are not limited to, polyadenylation signal sequences. Examples of polyadenylation signal sequences include, but are not limited to, Bovine growth hormone (BGH) poly(A), SV40 late poly(A), rabbit beta-globin (RBG) poly(A), thymidine kinase (TK) poly(A) sequences, and any variants thereof.

The viral vectors (e.g., AAV vector) can also have one or more restriction site(s) located near the promoter sequence to provide for the insertion of nucleic acid sequences encoding a mRNA/protein of interest.

The AAV vector used in the disclosed compositions and methods can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rh10, AAV.rh32/33, AAV.rh43, AAV.rh64Rl, rAAV2-retro, AAV-DJ, AAV-PHP.B, AAV-PHP.S, AAV-PHP.eB, or other engineered versions of AAV. In a particular form, the AAV vector is AAV9. These serotypes differ in their tropism, or the types of cells they infect, making AAV a very useful system for in certain embodiments transducing specific cell types. Typically, AAV vectors have a packaging limit of ˜4.7 kb. The AAV itself may be immunogenic, which in some settings, can be used for its adjuvant effects.

### Virus-Like Particles

In certain non-limiting embodiments, a virus-like particle (VLP) includes a disclosed encoded monkeypox virus antigen protein or subunit or other fragment thereof. VLPs are small particles that contain certain proteins from the outer coat of a virus and can be constructed to present these proteins as antigens on their coat. Typically, VLPs lack the viral components that are required for virus replication and thus represent a highly attenuated, replication-incompetent form of a virus. Thus, VLPs can be regarded as non-replicating, viral shells, derived from any of several viruses. The VLP can display a polypeptide (e.g., a monkeypox virus antigen protein encoded by a disclosed mRNA) that is analogous to that expressed on infectious virus particles and can elicit an immune response to the corresponding virus when administered to a subject.

VLPs can be derived from various viruses such as e.g. the hepatitis B virus or other virus families including Parvoviridae (e.g. adeno-associated virus), Retroviridae (e.g. HIV), and Flaviviridae (e.g. Hepatitis C virus). For a general review see Sorensen M R and Thomsen A R, APMIS 115 (11): 1177-93 (2007) and Guillén et al., Procedia in Vaccinology 2 (2), 128-133 (2010).

VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system.

Virus like particles and methods of their production are known and familiar to the person of ordinary skill in the art, and viral proteins from several viruses are known to form VLPs, including human papillomavirus, HIV (Kang et al., Biol. Chem. 380:353-64 (1999)), Semliki-Forest virus (Notka et al., Biol. Chem. 380:341-52 (1999)), human polyomavirus (Goldmann et al., J. Virol. 73:4465-9 (1999)), rotavirus (Jiang et al., Vaccine 17:1005-13 (1999)), parvovirus (Casal, Biotechnology and Applied Biochemistry, Vol 29, Part 2, pp 141-150 (1999)), canine parvovirus (Hurtado et al., J. Virol. 70:5422-9 (1996)), hepatitis E virus (Li et al., J. Virol. 71:7207-13 (1997)), and Newcastle disease virus.

The presence of VLPs following recombinant expression of viral proteins can be detected using conventional techniques known in the art. For example, the formation of VLPs can be detected by any suitable technique including techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation. VLPs can be isolated density gradient centrifugation and identified by characteristic density banding. See, for example, Baker et al. (1991) Biophys. J. 60:1445-1456; and Hagensee et al. (1994) J. Virol. 68:4503-4505; Vincente, J Invertebr Pathol., 2011; Schneider-Ohrum and Ross, Curr. Top. Microbiol. Immunol., 354:53073, 2012).

### Lipid Nanoparticles (LNPs)

In certain non-limiting embodiments, a disclosed mRNA or other disclosed polynucleotide (e.g., plasmid or vector) is formulated or encapsulated in a lipid nanoparticle. Non-limiting examples of lipid nanoparticles and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578, the contents of each of which are incorporated herein by reference in their entirety. Suitable lipid nanoparticle formulations are known in the art, see e.g., U.S. Pat. Nos. 9,950,065; 10,576,146; 10,485,884; 10,933,127; 10,703,789, and 10,702,600; which are hereby incorporated by reference in their entirety.

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In certain non-limiting embodiments, the lipid nanoparticle includes one or more cationic lipids (e.g., ionizable cationic lipid), one or more helper lipids, one or more sterols, one or more PEG-modified lipids, or a combination thereof. In certain embodiments, the lipid nanoparticle includes at least one cationic lipid (e.g., ionizable cationic lipid), at least one helper lipid, at least one sterol, and at least one PEG-modified lipid. In certain non-limiting embodiments, the cationic lipid is an ionizable cationic lipid, the helper lipid is a neutral lipid, and the sterol is cholesterol.

The ionizable cationic lipids, which are pH-sensitive, attract anionic nucleic acids to form the core of self-assembling nanoparticle to ensure high encapsulation. Ionizable lipids are protonated at low pH, which makes them positively charged, but they remain neutral at physiological pH. The pH-sensitivity of ionizable lipids is beneficial for mRNA delivery in vivo, because neutral lipids have less interactions with the anionic membranes of blood cells and, thus, improve the biocompatibility of lipid nanoparticles. This also eliminates a mechanism of toxicity seen with permanently cationic molecules. Trapped in endosomes, in which the pH is lower than in the extracellular environment, ionizable lipids are protonated and, therefore, become positively charged, which may promote membrane destabilization and facilitate endosomal escape of the nanoparticle and/or encapsulated mRNA or other nucleic acid.

In certain non-limiting embodiments, a lipid nanoparticle includes about 35 to 45% cationic lipid, about 40% to 50% cationic lipid, about 50% to 60% cationic lipid, or about 55% to 65% cationic lipid. In certain non-limiting embodiments, the cationic lipid is selected from 2,2-dilinoleyl-4-dimethylaminoethyl [1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy) heptadecanedioate (L319). Suitable ionizable cationic lipids also include, without limitation, PNI ionizable lipid, SM-102, ALC-0315, DLin-DMA, DLin-D-DMA, DLin-MC3-DMA, DLin-KC2-DMA, DODMA, 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), amino alcohol lipids and combinations thereof. Combinations of any of the foregoing cationic lipids can be used in various ratios.

Exemplary helper lipids include, but are not limited to, 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC) and 1,2-dioleoyl-sn-glycero-3-phosphocholine (DOPC). In certain non-limiting embodiments, the LNP includes from about 0.5% to about 15% on a molar basis of the helper lipid e.g., from about 3 to about 12%, from about 5 to about 10% or about 15%, about 10%, or about 7.5% on a molar basis. Examples of helper lipids include, but are not limited to, DSPC, POPC, DPPC, DOPE and SM.

The LNPs can include a sterol component. For example, a sterol component may be included to confer suitable physicochemical and biological behavior. Such a sterol component may be selected from cholesterol or its derivative e.g., ergosterol or cholesterolhemisuccinate, but it is in certain non-limiting embodiments cholesterol. Cholesterol is often used in lipidic formulations because it is generally recognized that the presence of cholesterol decreases their permeability and protects them from the destabilizing effect of plasma or serum proteins. In certain embodiments, the sterol is cholesterol. In certain non-limiting embodiments, the sterol is a cholesterol-PEG conjugate. Combinations of any of the foregoing sterols can be used in various ratios. In certain non-limiting embodiments, the LNP includes from about 5% to about 50% on a molar basis of the sterol (e.g., about 15 to about 45%, about 20 to about 40%, about 40%, about 38.5%, about 35%, or about 31% on a molar basis).

Exemplary PEG-modified lipids include, but are not limited to, R-3-[(ω-methoxy-poly(ethyleneglycol)2000)carbamoyl)]-1,2-dimyristyloxypropyl-3-amine (PEG-c-DOMG or PEG-DOMG), 1,2-dimyristoyl-racglycero-3-methoxypolyethylene glycol-2000 (PEG-DMG), PEG2000-DMG, 1,2-Distearoyl-sn-glycerol, methoxypolyethylene glycol (PEG-DSG), 1,2-Dipalmitoyl-sn-glycerol, methoxypolyethylene glycol (PEG-DPG), PEG-CDMA, mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, and mPEG-DPPE, and combinations thereof (further discussed in Reyes et al., J. Controlled Release, 107, 276-287 (2005), which is hereby incorporated by reference in its entirety). Combinations of any of the foregoing PEG-modified lipids can be used in various ratios.

In certain non-limiting embodiments, the LNPs include about 0.5% to 20% on a molar basis of the PEG or PEG-modified lipid (e.g., about 0.5 to 10%, about 0.5 to 5%, about 0.5%, about 1.5%, about 3.5%, or about 5% on a molar basis). In certain non-limiting embodiments, a PEG-modified lipid includes a PEG molecule of an average molecular weight of 2,000 Da. In certain non-limiting embodiments, a PEG-modified lipid includes a PEG molecule of an average molecular weight of less than 2,000 Da, for example around 1,500 Da, around 1,000 Da, or around 500 Da. The ratio of PEG in the lipid nanoparticle formulations may be increased or decreased and/or the carbon chain length of the PEG lipid may be modified from (e.g., from C14 to C18) to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0%, or 3.0% to 6.0% of the lipid molar ratio of PEG-modified lipid as compared to the cationic lipid, helper lipid and sterol.

In certain non-limiting embodiments, the LNP formulation may contain PEG-DMG 2000 (1,2-dimyristoyl-sn-glycero-3-phophoethanolamine-N-[methoxy (polyethylene glycol)-2000). In certain non-limiting embodiments, the LNP formulation may contain PEG-DMG 2000, a cationic lipid known in the art and at least one other component. In certain non-limiting embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example, the LNP formulation may contain PEG-DMG 2000, DLin-DMA, DSPC and cholesterol in a molar ratio of 2:40:10:48 (see, e.g., Geall et al., PNAS, 109 (36): 14604-9 (2012); PMID: 22908294).

In certain non-limiting embodiments, the lipid nanoparticle contains a lipid mixture in ratios of about 20-70% cationic lipid, 5-45% helper lipid, 20-55% cholesterol, 0.5-15% PEG-modified lipid; such as but not limited to about 20-60% ionizable cationic lipid, about 5-25% helper lipid, about 25-55% sterol, and/or about 0.5-15% PEG-modified lipid. In some certain embodiments, the lipid nanoparticle has a molar ratio of about 20-60% cationic lipid, about 5-25% helper lipid, 25-55% sterol, and 0.5-15% PEG-modified lipid. In certain non-limiting embodiments, the lipid nanoparticle includes about 25-75% of a cationic lipid, 0.5-15% of a helper lipid, 5-50% of a sterol, and 0.5-20% of PEG-modified lipid on a molar basis. In certain non-limiting embodiments, the lipid nanoparticle includes about 35-65% of a cationic lipid, 3-12% of a helper lipid, 15-45% of a sterol, and 0.5-10% of a PEG-modified lipid on a molar basis.

In certain non-limiting embodiments, the lipid nanoparticle has a mean diameter of about 10-500 nm, about 20-400 nm, about 30-300 nm, or about 40-200 nm. In certain non-limiting embodiments, the lipid nanoparticle has a mean diameter of about 20-100 nm, 40-100 nm, 50-100 nm, 50-150 nm, about 50-200 nm, about 80-100 nm or about 80-200 nm.

In certain non-limiting embodiments, the ratio of lipid to RNA (e.g., mRNA) in a lipid nanoparticle may be 5:1 to 20:1, 10:1 to 25:1, 15:1 to 30:1 and/or at least 30:1. In some certain embodiments, the ratio of lipid to mRNA in the disclosed lipid nanoparticles is in the range of about 5:1 to 20:1, inclusive. In some certain embodiments, the ratio of lipid to mRNA is 6:1. In certain non-limiting embodiments, the lipid to mRNA ratio is a molar ratio. For example, in some certain embodiments, the N:P molar ratio of a lipid nanoparticle containing mRNA is 6:1. The N:P ratio refers to the ratio of positively-chargeable polymer amine (N=nitrogen) groups to negatively-charged nucleic acid phosphate (P) groups. N:P ratio is an important physicochemical property of polymer-based gene delivery vehicles. The N:P character of a polymer/nucleic acid complex can influence many other properties such as its net surface charge, size, and stability.

Lipid nanoparticle formulations may be altered by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

The lipids in the LNPs can improve nanoparticle properties, such as particle stability, delivery efficacy, tolerability and biodistribution. For example, 1,2-distearoyl-sn-glycero-3-phosphocholine (DSPC), a phosphatidylcholine with saturated tails, has a melting temperature of ˜54° C. and a cylindrical geometry that allows DSPC molecules to form a lamellar phase, which stabilizes the structure of lipid nanoparticles. DSPC has been used in the mRNA-1273 and BNT162b2 COVID-19 vaccines. DOPE is a phosphoethanolamine with two unsaturated tails, which has a melting temperature of ˜30° C. and a conical shape 120. DOPE tends to adopt an inverted hexagonal H(II) phase, which destabilizes endosomal membranes and facilitates endosomal escape of lipid nanoparticles.

Cholesterol can enhance particle stability by modulating membrane integrity and rigidity. The molecular geometry of cholesterol derivatives can further affect delivery efficacy and biodistribution of lipid nanoparticles. For example, cholesterol analogues with C-24 alkyl phytosterols increase the in vivo delivery efficacy of LNP-mRNA formulations. Here, the length of the hydrophobic tails of the cholesterol analogues, the flexibility of sterol rings and the polarity of hydroxy groups impact delivery efficacy.

PEG-modified lipids can have multiple effects on the properties of lipid nanoparticles. The amount of PEG-modified lipids can affect particle size and zeta potential. PEG-lipids can further contribute to particle stability by decreasing particle aggregation, and certain PEG modifications prolong the blood circulation time of nanoparticles by reducing clearance mediated by the kidneys and the mononuclear phagocyte system

Once they reach target cells, lipid nanoparticles can be internalized by multiple mechanisms, including macropinocytosis and clathrin-mediated and caveolae-mediated endocytosis. The endocytic pathway depends on the properties of the nanoparticle and the cell type. Following cellular internalization, lipid nanoparticles are usually trapped in endosomal compartments. Thus, endosomal escape is crucial for effective mRNA or other nucleic acid delivery. It is believed that positively charged lipids may facilitate electrostatic interaction and fusion with negatively charged endosomal membranes, resulting in the leak of mRNA or other nucleic acid molecules into the cytoplasm. Endosomal escape can be increased by optimizing the pKa values of ionizable lipids. Furthermore, the properties of lipidic tails can affect endosomal escape of lipid nanoparticles. For example, some lipids with branched tails show enhanced endosomal escape compared with their counterparts with linear tails, owing to stronger protonation at endosomal pH. In addition, modulating the type (for example, DSPC and DOPE) and ratio of lipids may improve endosomal escape. See Hou X., et al., Nat Rev Mater., 1-17. (2021) doi: 10.1038/s41578-021-00358-0 for a discussion of the design of lipid nanoparticles for mRNA delivery and the physiological barriers and suitable administration routes for lipid nanoparticle-mRNA systems.

### Pharmaceutical Formulations

Also provided are pharmaceutical formulations including one or more of the more disclosed compositions (e.g., mRNA, other polynucleotide such as plasmids and vectors, optionally provided in a disclosed delivery vehicle (e.g., AAV, VLP, LNP) and one or more pharmaceutically acceptable carriers, diluents, and/or excipients.

In certain non-limiting embodiments, a pharmaceutical composition or formulation includes a disclosed lipid nanoparticle with one or more disclosed mRNAs encapsulated in the LNP, and a pharmaceutically acceptable carrier, diluent, or excipient. In certain non-limiting embodiments, a pharmaceutical composition or formulation includes a disclosed lipid nanoparticle encapsulating one or more disclosed polynucleotides (e.g., plasmids or vectors) encapsulated in the LNP, and a pharmaceutically acceptable carrier, diluent, or excipient. In certain non-limiting embodiments, a pharmaceutical composition or formulation includes a AAV vector containing a sequence encoding a disclosed mRNA and a pharmaceutically acceptable carrier, diluent, or excipient. In certain non-limiting embodiments, a pharmaceutical composition or formulation includes a VLP containing one or more encoded polypeptides (e.g., monkeypox virus antigen proteins or subunits or other fragment thereof) and a pharmaceutically acceptable carrier, diluent, or excipient.

Pharmaceutical compositions may optionally further include one or more additional active agents, e.g., therapeutic and/or prophylactic agents. General considerations in the formulation and/or manufacture of pharmaceutical agents may be found, for example, in Remington: The Science and Practice of Pharmacy 21st ed., Lippincott Williams & Wilkins, 2005.

The pharmaceutical compositions are in certain non-limiting embodiments sterile and contain an effective amount of the active compounds (e.g., mRNAs optionally encapsulated in LNPs, and optionally further agents) to generate the desired reaction or the desired effect. Pharmaceutical compositions are usually provided in a uniform dosage form and may be prepared in an appropriate manner. The pharmaceutical composition may for example be in the form of a solution or suspension. The pharmaceutical composition may include salts, buffer substances, preservatives, carriers, diluents and/or excipients all of which are in certain non-limiting embodiments pharmaceutically acceptable. Pharmaceutically acceptable refers to the non-toxicity of a material which does not interact with the action of the active component of the pharmaceutical composition.

The term “excipient” when used herein is intended to indicate all substances which may be present in a pharmaceutical composition and which are not active ingredients such as, e.g., carriers, binders, lubricants, thickeners, surface active agents, preservatives, emulsifiers, buffers, flavoring agents, or colorants. Pharmaceutically acceptable excipients include any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof.

In certain non-limiting embodiments, an excipient is approved for use in humans and for veterinary use. In certain non-limiting embodiments, an excipient is approved by United States Food and Drug Administration. In certain non-limiting embodiments, an excipient is pharmaceutical grade. In certain non-limiting embodiments, an excipient meets the standards of the United States Pharmacopoeia (USP), the European Pharmacopoeia (EP), the British Pharmacopoeia, and/or the International Pharmacopoeia.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate) and combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof. In certain non-limiting embodiments, suitable buffer substances include acetic acid in a salt, citric acid in a salt, boric acid in a salt, and phosphoric acid in a salt.

Exemplary lubricating agents include, but are not limited to, magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, etc., and combinations thereof.

The pharmaceutical compositions may be administered via any conventional route, such as by parenteral administration including by injection or infusion. Administration is in certain non-limiting embodiments parenterally, e.g., intravenously, intraarterially, subcutaneously, intradermally or intramuscularly. The term “parenteral administration” refers to the administration in a manner other than through the digestive tract, as by intravenous or intramuscular injection. Systemic administration is a route of administration that is either enteral, i.e., administration that involves absorption through the gastrointestinal tract, or parenteral. In certain non-limiting embodiments, the pharmaceutical compositions can be administered by a route selected from, for example, intramuscular, intradermal, subcutaneous, intravenous, intra-arterial, intra-articular, intraperitoneal, intranasal, sublingual, tonsillar, oropharyngeal, or other parenteral and mucosal routes. Actual methods for preparing administrable compositions will be known or apparent to those skilled in the art.

Compositions suitable for parenteral administration usually include a sterile aqueous or nonaqueous preparation of the active compound(s), which is in certain non-limiting embodiments isotonic to the blood of the recipient. Examples of compatible carriers and solvents are water, Ringer's solution, U.S.P., and isotonic sodium chloride solution. In addition, usually sterile, fixed oils are used as solution or suspension medium. For this purpose, any bland fixed oil can be employed including synthetic mono- or diglycerides. Fatty acids such as oleic acid can be used in the preparation of injectables.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Aqueous solutions of the pharmaceutical formulations may be packaged for use as is or lyophilized. Lyophilized preparations can be combined with a sterile solution prior to administration for either single or multiple dosing.

### Vaccines

The disclosed compositions, including pharmaceutical compositions can be suitable for use as vaccines. Thus, vaccines are provided herein. A vaccine is a biological preparation that improves or provides immunity to a particular disease or infectious agent. In certain non-limiting embodiments, a vaccine includes a disclosed pharmaceutical composition, optionally in combination with one or more adjuvants. In certain non-limiting embodiments, a vaccine includes a disclosed lipid nanoparticle encapsulating one or more mRNAs, optionally in combination with one or more adjuvants.

In certain embodiments, a vaccine includes a lipid nanoparticle encapsulating a mRNA which encodes one or more monkeypox virus envelope proteins or membrane proteins or cell surface binding proteins or subunits or other fragments thereof (e.g., A29, E8L, M1R, A35R and/or B6R) derived from monkeypox virus, or variants thereof.

In certain non-limiting embodiments, the vaccine can be multivalent, including mRNAs encoding proteins from multiple distinct proteins. For example, in certain non-limiting embodiments, a vaccine includes a lipid nanoparticle encapsulating multiple mRNAs which encodes distinct monkeypox virus antigen proteins or subunits or other fragments thereof (e.g., A29, E8L, M1R, A35R and/or B6R) derived from monkeypox virus, including variants thereof. In certain non-limiting embodiments, a vaccine includes a lipid nanoparticle encapsulating a single mRNA which encodes distinct monkeypox virus antigen proteins which are separated by cleavable linker sequences (e.g. 2A sequences) which result in the expression of multiple separate polypeptides from a single mRNA molecule. In such forms, the multivalent vaccine can induce immunity against several antigenic MPXV proteins concurrently.

In certain non-limiting embodiments, the mRNAs encoding proteins from multiple proteins are provided in equivalent amounts, e.g., 1:1 ratio, 1:1:1 ratio, etc.

Besides the LNP and encapsulated nucleic acid, the vaccine can also contain one or more excipients selected from sodium chloride, monobasic potassium phosphate, potassium chloride, dibasic sodium phosphate dihydrate, tromethamine, tromethamine hydrochloride, acetic acid, sodium acetate, and sucrose. In some certain embodiments, the vaccine includes sodium chloride, monobasic potassium phosphate, potassium chloride, dibasic sodium phosphate dihydrate, and sucrose. In some certain embodiments, the vaccine includes tromethamine, tromethamine hydrochloride, acetic acid, sodium acetate, and sucrose.

The disclosed vaccines can further include, or may be administered in combination with, one or more adjuvants. Adjuvants describe compounds which prolong, enhance, accelerate, and/or exacerbate an immune response. Various mechanisms are possible in this respect, depending on the type of adjuvants used. In certain non-limiting embodiments, the vaccines include, or are administered in combination with, one or more adjuvants. In certain non-limiting embodiments, the vaccines do not include, or are not administered in combination with, one or more adjuvants.

Non-limiting examples of suitable adjuvants include cytokines, such as monokines, lymphokines, interleukins or chemokines (e.g., IL-1, IL-2, IL-3, IL-4, IL-5, IL-6, IL-7, IL-8, IL-9, IL-10, IL-12, INFα, INF-γ, GM-CSF, LT-α), lipopolysaccharides (LPS), CD40 ligands, GP96, dsRNA, CpG oligodeoxynucleotides, growth factors (e.g. hGH), aluminium hydroxide, Freund's adjuvant or oil such as Montanide®, In certain non-limiting embodiments Montanide® ISA51, lipid-A and derivatives or variants thereof, oil-emulsions, saponins, and Pam3Cys. These adjuvants have the advantage in that they help to stimulate the immune system in a non-specific way, thus enhancing the immune response to a pharmaceutical product.

### Methods of Making

Methods of making the disclosed mRNAs, other polynucleotides, and compositions and pharmaceutical formulations thereof are provided.

Polynucleotides may be prepared according to any available technique including, but not limited to chemical synthesis, enzymatic synthesis, which is generally termed in vitro transcription (IVT) or enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

The process of design and synthesis of the primary constructs of the disclosure generally includes the steps of gene construction, mRNA production (either with or without modifications) and purification. In the enzymatic synthesis method, a target polynucleotide sequence encoding the polypeptide of interest is first selected for incorporation into a vector which will be amplified to produce a cDNA template. Optionally, the target polynucleotide sequence and/or any flanking sequences may be codon optimized. The cDNA template is then used to produce mRNA through in vitro transcription (IVT).

mRNAs may be made using standard laboratory methods and materials. In certain non-limiting embodiments, mRNAs are produced by in vitro transcription of a linear or circularized DNA template (e.g., plasmid or other expression vector) containing sequences encoding the mRNAs. Plasmids or other expression vectors can be linearized by methods known in the art, such as restriction enzymes. The linearization reaction may be purified using methods including, for example Invitrogen's PURELINK™ PCR Micro Kit (Carlsbad, Calif.), and HPLC based purification methods such as, but not limited to, strong anion exchange HPLC, weak anion exchange HPLC, reverse phase HPLC (RP-HPLC), and hydrophobic interaction HPLC (HIC-HPLC) and Invitrogen's standard PURELINK™ PCR Kit (Carlsbad, Calif.).

The DNA template may be transcribed using an in vitro transcription (IVT) system. The system typically includes a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The DNA template may be removed using methods known in the art such as, but not limited to, treatment with Deoxyribonuclease I (DNase I). RNA clean-up may also include a purification method such as, but not limited to, AGENCOURT® CLEANSEQ® system from Beckman Coulter (Danvers, Mass.), and HPLC based purification methods such as, but not limited to, strong anion exchange HPLC, weak anion exchange HPLC, reverse phase HPLC (RP-HPLC), and hydrophobic interaction HPLC (HIC-HPLC).

The mRNA construct may undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.), optionally with a 2′-O methyl-transferase. If a poly(A) tail is not encoded in the DNA template and thus absent from the mRNA transcript, a poly(A) tailing reaction may be performed by methods known in the art, such as, but not limited to, poly(A) Polymerase mediated tailing.

Subsequently, mRNA clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified mRNA” refers to one that is separated from at least one contaminant. Thus, a purified polynucleotide (e.g., DNA or RNA) is present in a form or setting different from that in which it is found in nature, or a form or setting different from that which existed prior to subjecting it to a treatment or purification method.

The disclosed mRNAs can be formulated by mixing the mRNA with LNPs at a set ratio. Methods for synthesis of LNPs are known in the art. See for example, WO 2010/054401; Heyes et. al, J. Control Release, 107, 276-287 (2005); Semple et. al, Nature Biotechnology, 28, 172-176 (2010); Love et. al, PNAS, 107, 1864-1869 (2010) and Akinc et. al, Nature Biotechnology, 26, 561-569 (2008), all of which are hereby incorporated by reference in their entirety.

In certain non-limiting embodiments, solutions of the lipids/sterols forming the LNPs are combined in the appropriate molar ratio and diluted with ethanol to a final desired lipid concentration. Solutions of mRNA at a desired concentration in water can be diluted in sodium citrate buffer. Formulations of the lipid and mRNA can be prepared by combining the synthesized lipid solution with the mRNA solution at a desired total lipid to mRNA ratio. The formulations can be dialyzed one or more times against phosphate buffered saline (PBS) to remove the ethanol and to achieve buffer exchange. The resulting nanoparticle suspension can be filtered and stored as appropriate or used in accordance with the disclosed methods.

### Methods of Use

Methods of using the disclosed mRNAs, other polynucleotides, compositions and pharmaceutical formulations thereof, and vaccines are also provided herein, as illustrated elsewhere herein.

### Protein Expression

In certain non-limiting embodiments, the mRNAs and other polynucleotides can be used in methods to express and/or purify a desired protein or peptide, such as a monkeypox virus antigen protein or subunit or other fragment thereof. For example, in certain non-limiting embodiments, a method of producing a recombinant monkeypox antigen protein (e.g., A29, E8L, M1R, A35R and/or B6R) involves introducing an appropriate disclosed mRNA or other disclosed polynucleotide (e.g., plasmid, expression vector) to a host cell under conditions sufficient for expression thereof, thereby producing the recombinant monkeypox antigen protein.

### Vaccination

The disclosed pharmaceutical compositions and vaccines can be used in methods of inducing an immune response or vaccination. Typically, the immune response is against a monkeypox virus, including antigens thereof, such as a monkeypox antigen protein or subunit or other fragment thereof. In certain non-limiting embodiments, a method of inducing an immune response in a subject involves administering to the subject a disclosed vaccine in an effective amount to generate the immune response.

In certain non-limiting embodiments, the immune response is a T cell response. In certain non-limiting embodiments, the immune response is a B cell response. In certain non-limiting embodiments, the immune response involves both a T cell and B cell response. In certain non-limiting embodiments, the immune response involves a neutralizing antibody response specific to the monkeypox antigen protein or subunit or other fragment thereof. In certain non-limiting embodiments, the immune response inhibits monkeypox virus infection in the subject. In certain non-limiting embodiments, the immune response inhibits replication of the monkeypox virus in the subject. The immune response can be a protective immune response, for example a response that inhibits subsequent infection with the virus (e.g., MPXV). Elicitation of the immune response can also be used to treat or inhibit viral infection and illnesses associated with the virus, such as monkeypox.

Administration of a disclosed vaccine can be for prophylactic or therapeutic purpose. When provided prophylactically, the vaccine is provided in advance of any symptom, for example, in advance of infection. The prophylactic administration serves to prevent or ameliorate the course of any subsequent infection. When provided therapeutically, the vaccine is provided at or after the onset of a symptom of infection, for example, after development of a symptom of monkeypox virus infection or after diagnosis with monkeypox virus infection. The vaccine can thus be provided prior to the anticipated exposure to the virus (e.g., monkeypox virus) so as to attenuate the anticipated severity, duration or extent of an infection and/or associated disease symptoms, after exposure or suspected exposure to the virus, or after the actual initiation of an infection.

In certain non-limiting embodiments, the subject being vaccinated has been exposed to, is infected with, or is at risk of infection by the monkeypox virus. In certain non-limiting embodiments, the subject is immunocompromised. In certain non-limiting embodiments, the subject is human.

### Effective Amounts and Dosage Regimens

The pharmaceutical compositions, vaccines and other compositions described herein are administered in effective amounts. For example, the vaccine is provided to a subject in an amount effective to induce or enhance an immune response. The effective amount achieves a desired response or effect alone or together with further doses. In the case of treatment of a particular disease or of a particular condition, the desired response can be inhibition of the course of the disease. This can include slowing down the progress of the disease and, in particular, interrupting or reversing the progress of the disease. The desired response in a treatment of a disease or of a condition may also be delay of the onset or a prevention of the onset of said disease or said condition.

An effective amount of an agent or composition (e.g., vaccine) can depend on the disease indication, the severeness of the disease, the individual parameters of the subject (e.g., age, physiological condition, size and weight, fitness, extent of symptoms, susceptibility factors, and the like), the duration of treatment, the type of an accompanying therapy (if present), the specific route of administration, as well as the specific pharmacology of the composition for eliciting the desired activity or biological response in the subject, and similar factors. Accordingly, the doses administered of the vaccines may depend on various of such parameters. In certain non-limiting embodiments, the vaccine is administered in an effective amount to elicit a desired immune response, for example, a T cell and/or B cell response, and/or a neutralizing antibody response.

In certain non-limiting embodiments, a vaccine can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof.

Vaccination can involve one or more doses or administrations of the vaccines. In certain non-limiting embodiments, a single dose of a vaccine is administered. In certain non-limiting embodiments, two or more doses of a vaccine are administered. The two or more doses can be administered on different days, for example, about 14-28 (e.g., 14, 21, or 28) days apart. In certain non-limiting embodiments, the two or more doses can be administered 1, 2, 3, 4, 5, 6 or more months apart.

In certain non-limiting embodiments, each administration of the vaccine provides a dose of about 1 μg, 3 μg, 10 μg, 25 μg, 30 μg, or 100 μg of mRNA. In certain non-limiting embodiments, the effective amount of the vaccine is a total dose (e.g., over multiples administrations) of about 1-500 μg of mRNA, inclusive.

Dosage regimens can be adjusted to provide an optimum prophylactic or therapeutic response. A vaccine can be used in coordinate (or prime-boost) vaccination protocols or combinatorial formulations. In certain non-limiting embodiments, coordinate immunization protocols employ separate vaccines, each directed toward eliciting an anti-viral immune response, such as an immune response to monkeypox virus and variants thereof. Separate vaccines that elicit an antiviral immune response can be combined in a polyvalent vaccine composition administered to a subject in a single immunization step, or they can be administered separately (in monovalent vaccine compositions) in a coordinate (or prime-boost) immunization protocol. There can be several boosts, and each boost can be a vaccine presenting a different immunogen (e.g., monkeypox antigen protein or subunit or other fragment thereof) from the same or different virus.

The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. In certain non-limiting embodiments, the boost can be administered about two, about three to eight, or about four weeks following the prime, or about several months after the prime. In certain non-limiting embodiments, the boost can be administered about 5, about 6, about 7, about 8, about 10, about 12, about 18, about 24, months after the prime, or more or less time after the prime. Periodic additional boosts can also be used at appropriate time points to enhance the subject's immune memory. The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional vaccine d and/or the vaccination parameters can be modified in a fashion expected to potentiate the immune response.

### Routes of Administration

The vaccines and other pharmaceutical compositions may be administered by any suitable route. Administration can be local or systemic. Exemplary routes of administration include, but are not limited to, enteral, gastroenterol, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal (through the eye), intracavernous injection (into the base of the penis), intravaginal, intrauterine, transdermal (diffusion through the intact skin for systemic distribution), transmucosal (diffusion through a mucous membrane), insufflation (snorting), and sublingual. In certain non-limiting embodiments, administration is via intradermal or intramuscular injection, or via oral, intranasal or intratracheal administration. For example, administration can be via drops or sprays. In certain embodiments, administration is via intramuscular injection.

### Kits

The disclosed polynucleotides, reagents, compositions, and other materials can be packaged together in any suitable combination as a kit useful for performing, or aiding in the performance of, the methods. It is useful if the components in a given kit are designed and adapted for use together in the method.

For example, kits including vaccines or other compositions for administration to a subject, may include a pre-measured dosage of the composition in a sterile needle, ampule, tube, container, or other suitable vessel. The kits may include instructions for dosages and dosing regimens. In certain non-limiting embodiments, the vaccine compositions are lyophilized. The kit may further include agents (e.g., saline, a buffered solution) and instructions to form a formulation for administration. The instructions may specify suitable storage conditions for the kit and components thereof.

Also provided are kits for protein production. Such kits can include a disclosed polynucleotide (e.g., plasmid or other expression vector), viruses, virus-like particles, and/or instructions for use. The kit can further include reagents and instructions for transfection or transduction of recipient cells.

### EXPERIMENTAL EXAMPLES

The disclosure is now described with reference to the following Examples. These Examples are provided for the purpose of illustration only, and the disclosure is not limited to these Examples, but rather encompasses all variations that are evident as a result of the teachings provided herein.

The materials and methods employed in these experiments are now described.

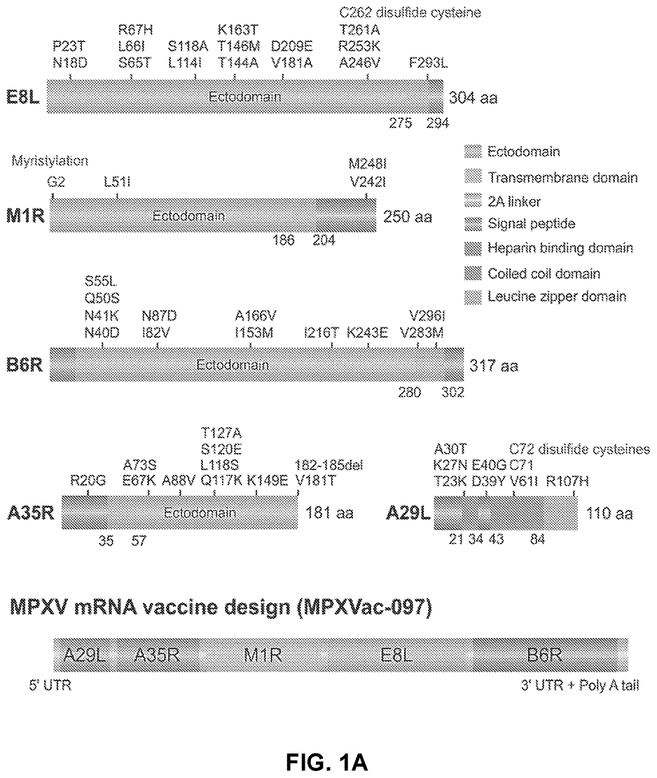

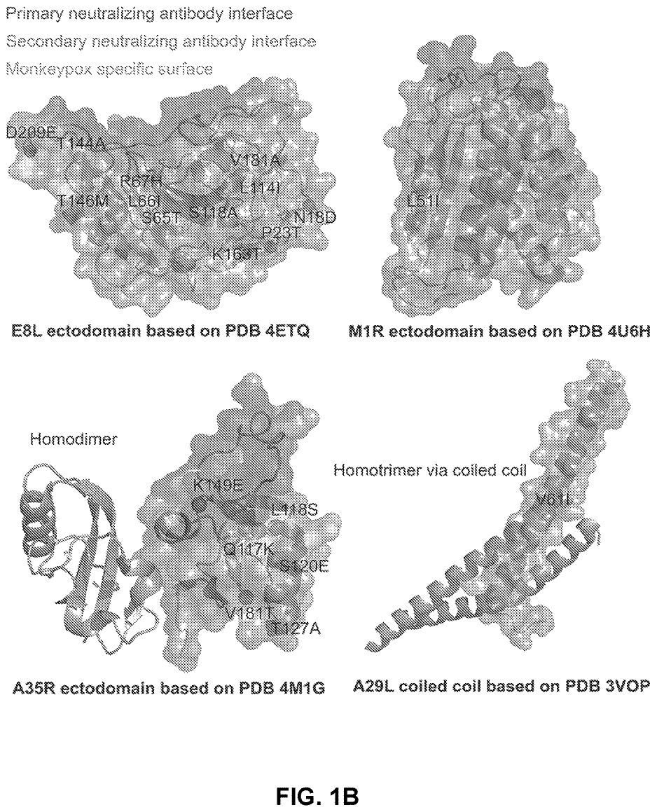

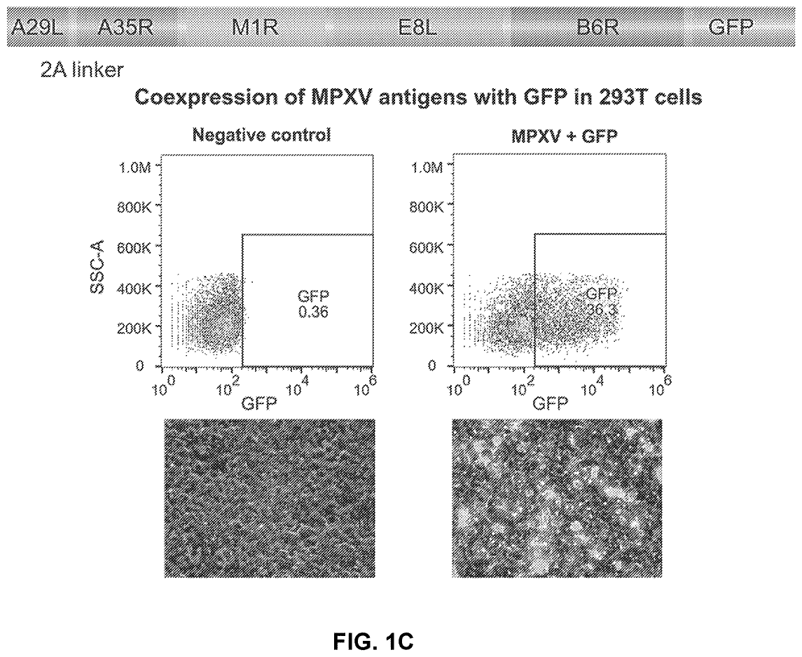

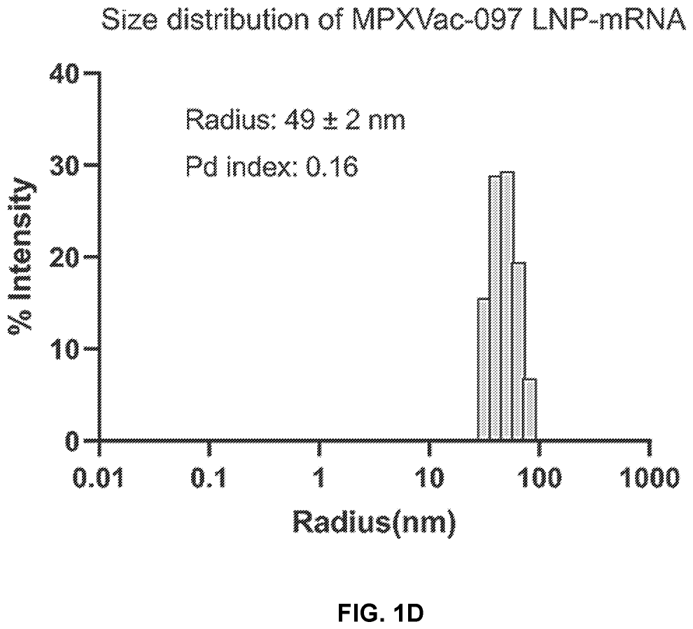

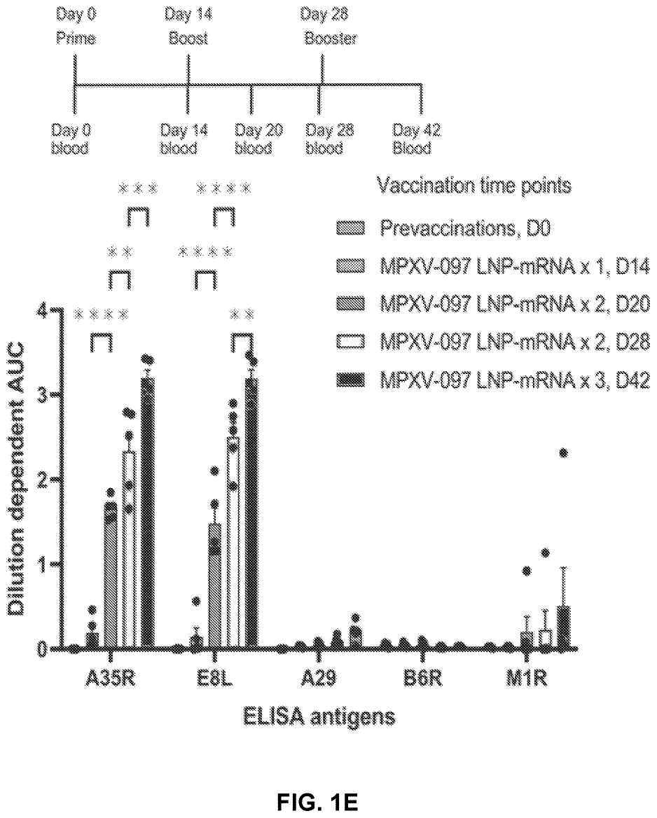

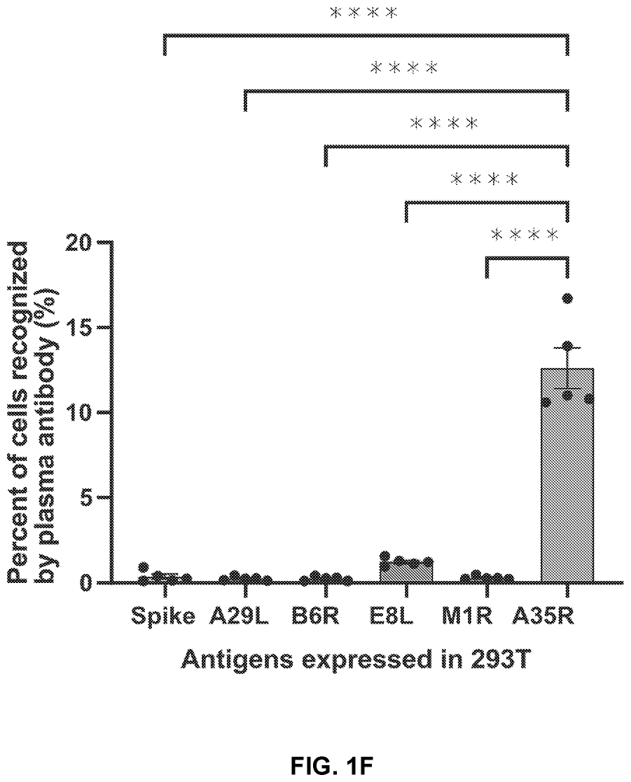

Plasmid cloning. The monkeypox antigen amino acid sequences used in the mRNA vaccine design were based on the genome of confirmed monkeypox case identified in Massachusetts in May 2022 (Genbank ON563414.3). The five antigen cDNAs were codon optimized, synthesized and inserted to mRNA vector with 5′,3′ UTRs and poly A tail as flanking sequences. The MPXVac-097 mRNA vaccine vector was cloned through connecting five antigen cDNAs with 2A linkers as shown in FIG. 1A. A reporter vector with GFP appended to the 3′ end of the mRNA vaccine vector, MPXV-PentAg-GFP, was designed to validate the successful translation of mRNA in mammalian cells (FIG. 1D).

SWISS homology modeling. Homologs of four out of five monkeypox antigens were found in PDB database and were used to build the homology structures on SWISS homology-modeling server (SWISS-MODEL (expasy.org)). The homology model was visualized in Pymol and was used to display neutralizing antibody epitopes and sites that are different between monkeypox and MVA.

Cell Culture. HEK293T cells (ATCC, CRL-3216) were cultured in Dulbecco's modified Eagle's medium (DMEM, Thermo fisher) with 10% hea-inactivated fetal bovine serum (FBS, Hyclone). Cells were split every other day at a split ratio of 1:4 or when confluency reaches over 95%.

Monkeypox antigen expression in 293T cells. The Lipo3000 transfection system (Thermo Fisher, Cat. No. L3000015) was applied to transfect 293T cells with a reporter vector that contains five monkeypox antigens in tandem and a 3′-end GFP. The 293T cells were seeded on the 6-well plates one day before transfection and transfection was performed according to manufacturer's protocol. Two days after transfection, cells were imaged under fluorescence microscope and were subsequently subject to flow cytometry for quantification of cells co-expressing five monkeypox antigens and GPF.

Cell surface monkeypox antigen staining using mice plasma antibody. The same lipo3000 transfection approach was also applied to express individual monkeypox antigen in 293T cells. The 293T cells expressing monkeypox antigen were trypsin treated, washed with 2% FBS in PBS once and resuspended in flow cytometry staining buffer (Thermo Fisher, Cat. No. 00-4222-26). Resuspended cells were incubated on ice for 20 min with 1:100 diluted plasma from mice vaccinated with two doses of MPXV LNP-mRNA. After incubation, cells were washed once with flow cytometry staining buffer and incubated on ice for 20 min with 1:100 diluted anti-mouse IgG antibody conjugated to PE fluorophore (Thermo Fisher, Cat. No. P-852). After the final wash with flow staining buffer, PE-positive cells were detected by flow cytometry using Attune focusing cytometer (Attune N×T Software v3.1). FlowJo v.10.7.1 was used for flow cytometry data analysis.

In vitro mRNA transcription and lipid nanoparticle preparation. To produce mRNA, the MPXVac-097 vaccine candidate construct encoding five monkeypox antigens in tandem was in vitro transcribed from linear DNA template using HiScribe T7 ARCA mRNA kit (NEB, Cat. No. E2060S) with 50% replacement of uridine by N1-methyl-pseudouridine (Tirlink, Cat. No. N-1081-5). The transcribed mRNA was purified using Monarch RNA Cleanup Kits (NEB, Cat. No. T2040L) and kept in-20 until further use.

The prepared mRNA was diluted in 25 mM sodium acetate buffer at pH 5.2 and mixed with lipid mixture in ethanol using NanoAssemblr™ Ignite™ instrument (Precision Nanosystems). The lipid mixture is composed of ALC-0315, ALC-0159, DSPC and cholesterol at a mixing ratio as previously described. The MPXV LNP-mRNA buffer was exchanged to phosphate buffered saline (PBS) using 100 kDa Amicon filter (Macrosep Centrifugal Devices 100 K, 89131-992). The mRNA encapsulation rate and mRNA concentration were determined by Quant-iT™ RiboGreen™ RNA Assay (Thermo Fisher). The size distribution of LNP-mRNA sample was characterized by dynamic light scattering (DynaPro NanoStar, Wyatt, WDPN-06). Sucrose was added to the final LNP-mRNA sample before storing it in-80 freezer.

Mouse immunization. 8-10 week-old female C57BL/6Ncr mice purchased from Charles River were vaccinated with two doses of 8 μg MPXVac-097 LNP-mRNA vaccine candidate on day 0 (prime), day 14 (boost) and day 28 (booster). The mice were maintained at an ambient room temperature, 40-60% humidity and a 14 h: 10 h day/night cycle. Retro-orbital blood was drawn using heparinized micro capillary tubes (Fisher, Cat. No. 22362566) and collected in lithium heparin microtainers (Fisher, Cat. No. 13-680-62) just before prime, boost and booster on day 0, 14 and 28. Additional blood samples were collected in the same way on day 20 (6 days post boost) and 42 (14 days post booster).

Isolation of plasma and peripheral blood mononuclear cells (PBMCs) from blood. The collected blood was 1:1 diluted with 2% FBS and was added to 7 ml of Lymphoprep™ density gradient medium in SepMate-15 tubes (StemCell Technologies). The red blood cells, PBMCs and plasma were isolated from blood by centrifugation at 1200×g for 20 min. After centrifugation, approximately 200 to 300 ul plasma was collected from the surface layer and the PBMCs in the remaining solution at the top were poured to a new tube. The separated PBMCs were washed with 2% FBS and sample's mRNA was extracted using RNeasy Plus Mini Kit (Qiagen).

ELISA. Commercial monkeypox antigens used in ELISA include E8L (AcroBiosystems, E8L-M52H3), M1R (Sino Biological, 40904-V07H), B6R (Sino Biological, 40902-V08H), A35R (Sino Biological, 40886-V08H), and A29L (Sino Biological, 40891-V08E). Recombinant antigens at 3 μg/ml in PBS were coated on the 384-well microplate (Fisher, Cat. No. 07-000-877) in cold room overnight. On next day, the plate was washed three time with PBST (0.05% Tween-20) on 50TS microplate washer (Fisher Scientific, NC0611021), and was blocked with 0.5% BSA in PBST at room temperature for one hour. Plasma was twofold serially diluted with PBS at a starting dilution of 1:500. The plasma diluent was added to the plate and incubated at room temperature for one hour. After washing with PBST five time, the plate was incubated with anti-mouse IgG (H+L) secondary antibody (Fisher, A16072) at room temperature for one hour. The secondary antibody with horse radish peroxidase was 1:2500 diluted in 0.5% BSA blocking buffer before adding to the microplate. The plate was washed five times with PBST and developed with tetramethylbenzidine substrate (Biolegend, 421101). After 20 min at room temperature, the reaction was stopped with 1M phosphoric acid and OD450 was measured by multimode microplate reader (PerkinElmer EnVision 2105, Envision Manager v1.13.3009.1401). The dilution-dependent area under curves (AUC) was calculated from AUC subtracted by the background AUC, which is the product of log dilution difference and end dilution OD450.

Bulk TCR sequencing of PBMCs. Bulk TCR library was prepared using 200 ng mRNA extracted from PBMCs collected on day 0 (pre-vaccination) and day 20 (post boost) mice as described above. The SMARTer Mouse TCR a/b Profiling Kit (Takara, Cat. No. 634403) was used to amplify both TCRα and TCRβ sequences. The purified amplicon concentration and purity was determined using D5000 high sensitivity tapes on TapeStation (Agilent). Equal amount of TCR amplicons from each sample was pooled and diluted with water to get a 2 nM library. The pooled library was denatured, mixed with 5% PhiX and sequenced on MiSeq (Illumina) using MiSeq V3 2×300 cycle kit (Illumina).

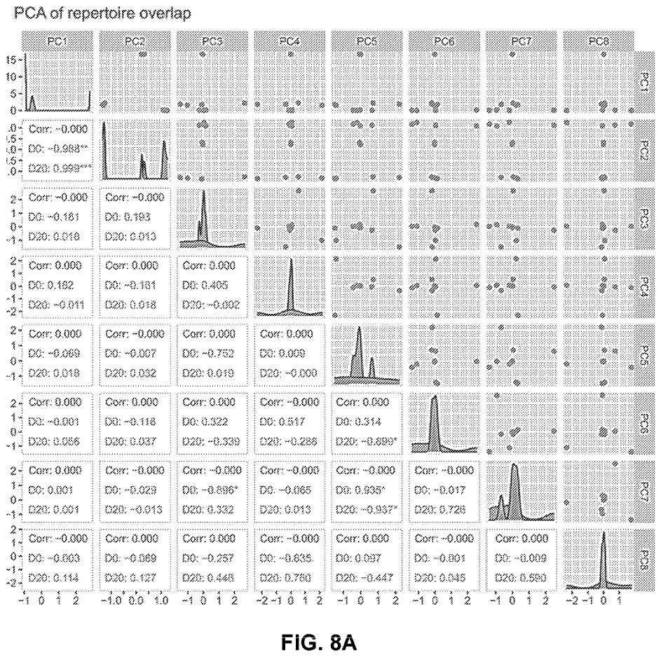

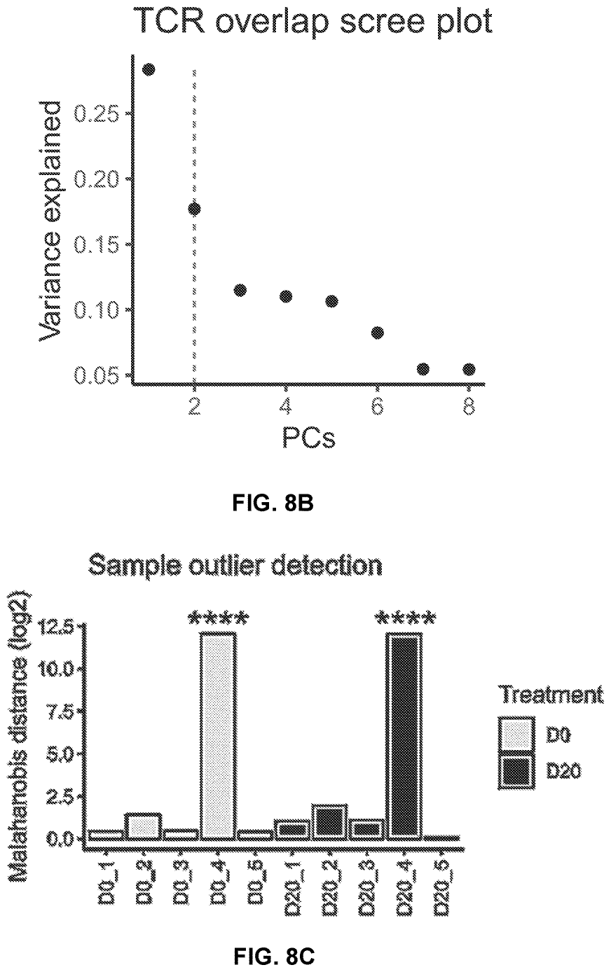

VDJ sequencing data analysis. The bulk VDJ sequencing data was pre-processed on MiSeq local run manager to trim adaptors and separate reads based on sample index. The reads with an average quality score less than 30 were removed from downstream analysis. Clonotypes were called using MiXCR v2.1.5 with the recommended settings for 5′ RACE (RNA alignment to V gene transcripts with P region). The output was further processed with Immunarch v0.6.6 R package for statistical analyses using standard analysis pipelines. The initial samples were n=5 independent mouse samples, assessed pre-vaccination and post-boost (labeled DO and D20, respectively) in paired analyses. Samples were assessed for outliers by a multi-step process, (1) comparing the repertoire overlap (amino acid sequence and V gene) between samples using the Morisita method, separately for TRA AND TRB, (2) performing principal components analysis on combined TRA and TRB overlap information (Morisita indices), (3) determining the optimal PCs for subsequent analysis (details below), (4) calculating Mahalanobis sample distances using the optimal number of PCs, and (5) calculating p values using a chi-squared distribution (2 degrees of freedom for 2 PCs) and correcting for multiple testing using the FDR method (FIGS. 8A-8C). The optimal number of PCs was chosen as two using the elbow plot method, and the choice was supported by PC scatter plots that demonstrated that PC1 best explained outlier samples, PC2 best explained the treatment differences, while Pearson correlation analyses of PC1 and PC2 demonstrated the best treatment specific correlation. The filtering parameter excluded two TCR libraries, one from each group (D0 or D20), and the final TCR analysis consists of four samples from each group (n=4 each).

### Example 1: Development of a Multivalent MPXV mRNA Vaccine

The 2022 monkeypox virus (MPXV) variants causing the outbreak form the lineage B.1 branch that belongs to MPXV clade 3. Because of high sequence identity, immunity elicited by attenuated vaccinia vaccine can provide protection from monkeypox and smallpox. Both MPXV and vaccinia virus belong to Orthopoxvirus genus, which also includes variola virus, the pathogen of smallpox. Proteomic analysis of antibody response to MPXV infection and vaccinia vaccination revealed a number of immunodominant envelope proteins. Among them, vaccinia A27L, D8L, H3L, LIR, A33R and B5R have been identified as targets of neutralizing antibodies. Polyvalent DNA vaccines combining vaccinia antigens A27L, D8L, LIR, A33R and B5R protected macaques and mice from lethal MPXV challenge. The MPXV equivalent antigens of vaccinia A27L, D8L, LIR, A33R and B5R are A29L, E8L, M1R, A35R and B6R respectively. A number of important questions arise when exploring opportunities of developing a MPXV based vaccine. For example, how different are the MPXV and MVA antigens? Could an mRNA vaccine against MPXV elicit potent antibody response? Would an mRNA vaccine encoding these antigens be generally safe?

The mRNA vaccine technology has catalyzed the tremendous success of COVID mRNA vaccine, which has proven to be safe and effective across the globe in both initial clinical trials and real-world data. It is a versatile platform that can be adopted to develop mRNA vaccines against other infectious diseases, including monkeypox. Although MVA-based vaccine has less side effect than ACAM2000, its >=3 grade adverse event rate is 7.7% in recipients. In contrast, the >=3 grade adverse event rate of COVID mRNA vaccine is similar to placebo group (1.5% in vaccine group vs. 1.3% in placebo group) 19. The MVA vaccine contains around 200 proteins, many of which are not immunogenic and likely associated with undesired side effects. Removal of undesired components in MPXV vaccines would improve its safety profile, which is the case for MVA that lost large fragments of genome during attenuation. Simplification of MPXV vaccine could facilitate rapid vaccine optimization. In addition, the manufacturing of mRNA vaccine can be achieved in vitro independent of complex cell culture as opposed to inactivated or attenuated vaccine; and is easily scalable by biochemical reactions. These features together make mRNA vaccine promising to enable the world to quickly fill the gap between vaccine supply and demand during a disease outbreak. The shortage of monkeypox vaccine and adaptability of mRNA vaccine prompt the idea of developing MPXV-targeting mRNA vaccine. However, whether the mRNA-based MPXV antigens can elicit significant immune response in vivo remains a critical question to be answered.

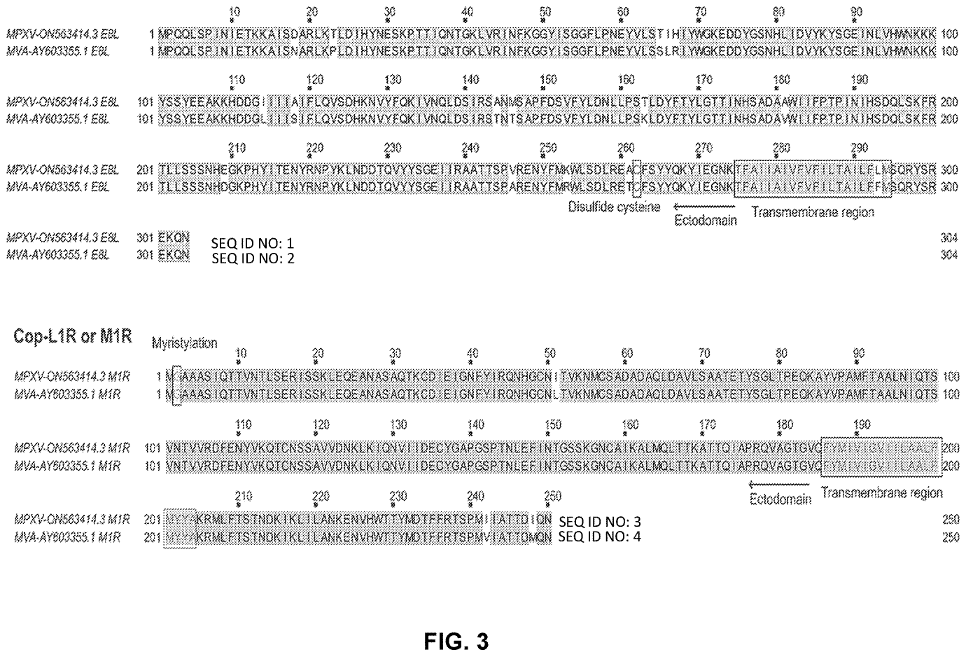

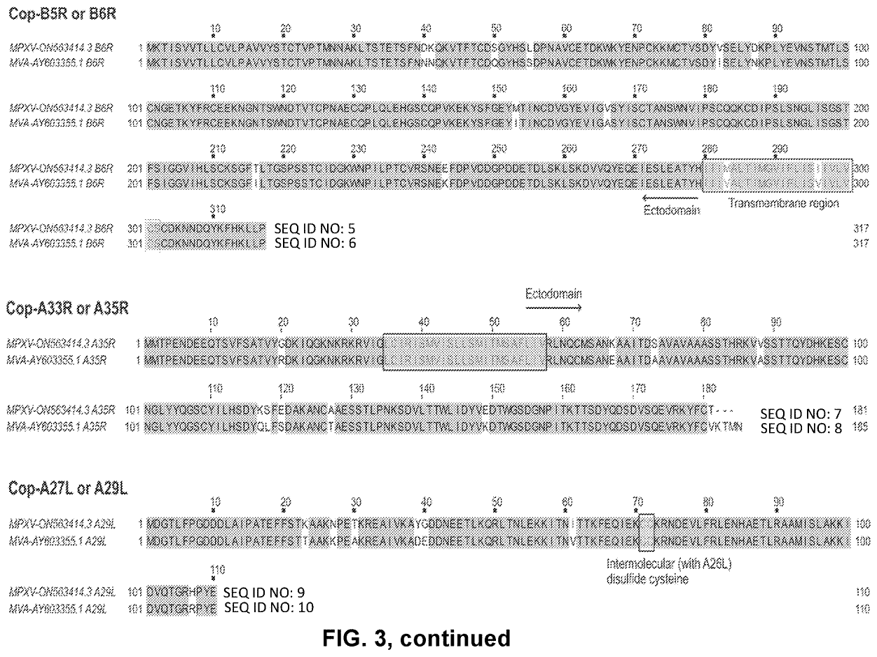

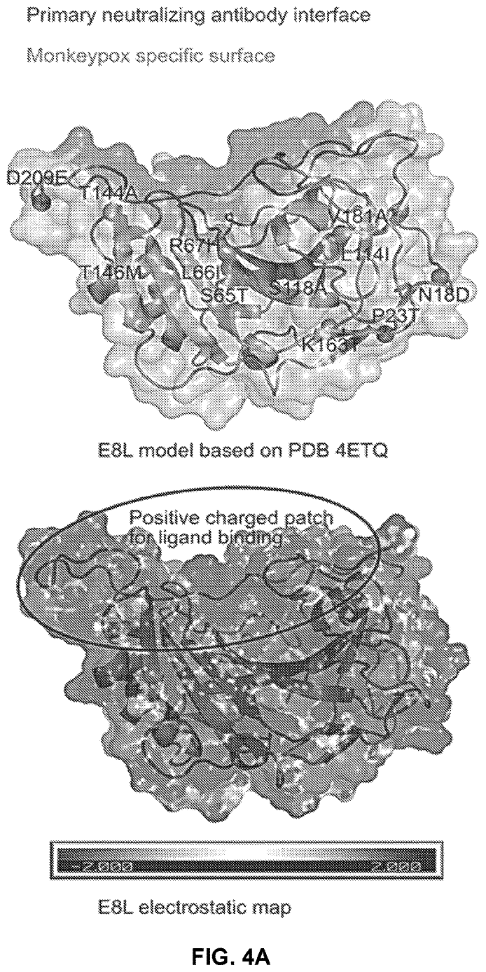

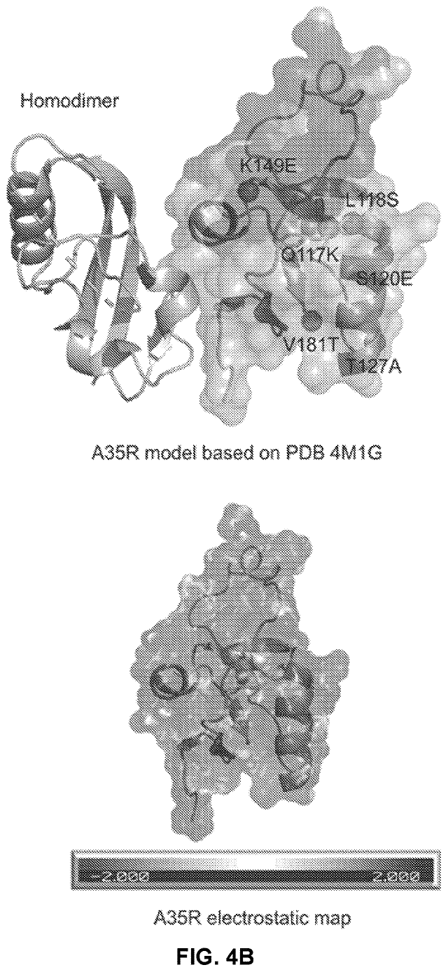

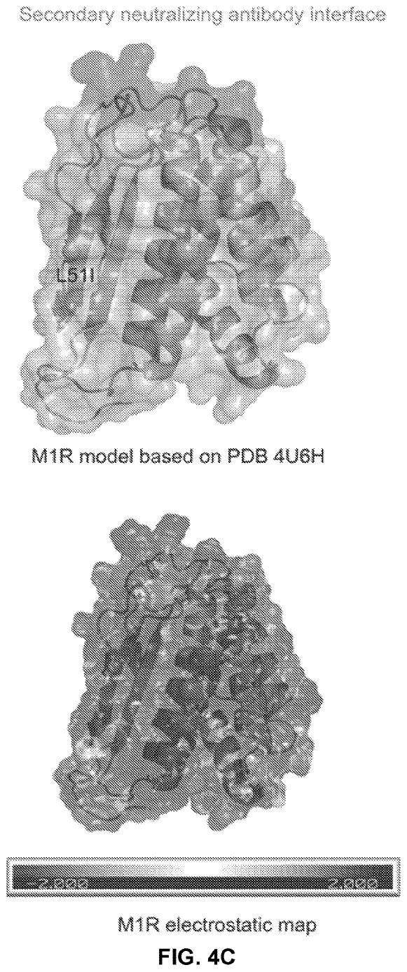

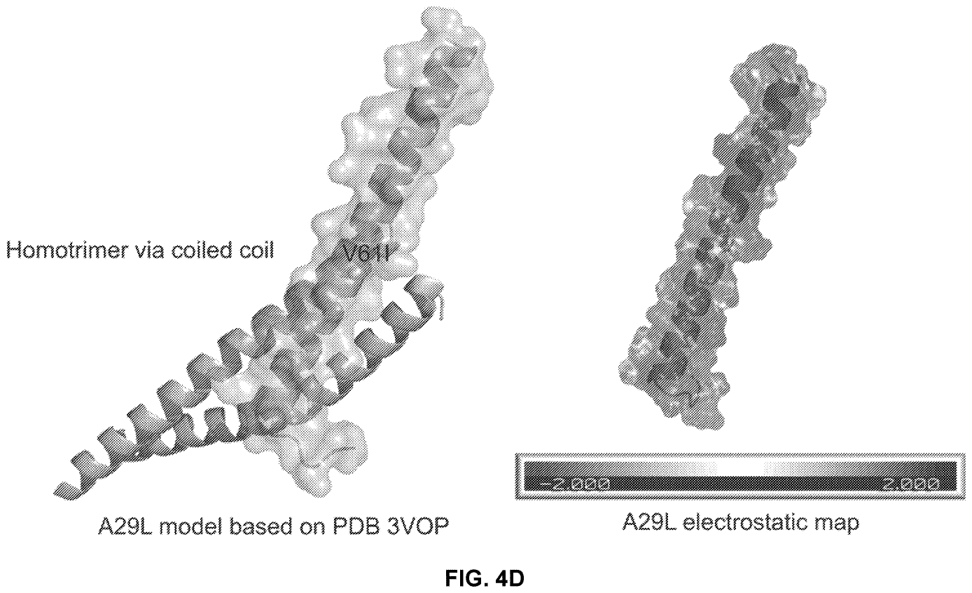

The studies disclosed herein designed a polyvalent mRNA vaccine candidate, MPXVac-097, against 2022 MPXV and initiated the testing of its antibody response as well as T cell receptor repertoire in vaccinated mice. Five MPXV antigens with prior evidence as neutralizing antibody targets, A29, E8L, M1R, A35R and B6R, were used in this vaccine design (FIG. 1A). They were linked in tandem by 2A peptides and codon optimized based on the protein sequence of 2022 MPXV case identified in USA in May 2022. Their sequence alignment with homologs of MVA showed over 90% sequence identity, and identified dozens of species-specific residues (FIGS. 1A and 3). M1R antigen is largely conserved between MPXV and MVA, while the other four antigens contain 4-6% substitutions. Among these substitutions, K149E in A35R and T144A, S65T, L66I, R67H in E8L are located on reported neutralizing antibody interface and may lead to immune evasion of MVA neutralizing antibodies (FIG. 1B). The MPXV mRNA vaccine adopts the circulating MPXV sequence and can avoid this antigen mismatch. Interestingly, to prevent host cell interaction, the neutralizing antibody of E8L recognizes and blocks the ligand binding site which contains a large positive-charge patch to accommodate the negative charged chondroitin sulfate ligand of host cells (FIG. 4).

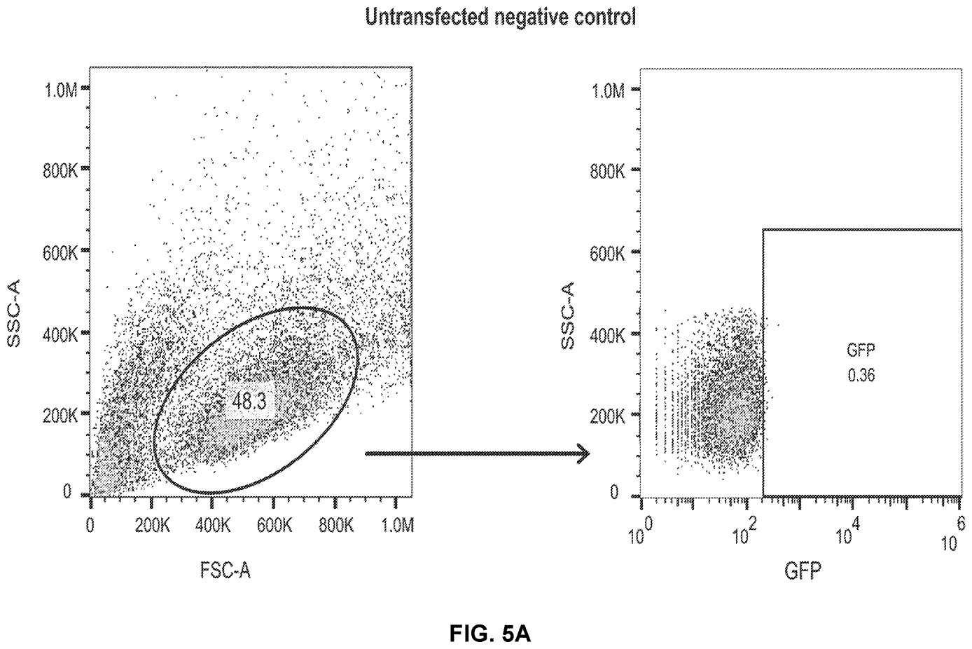

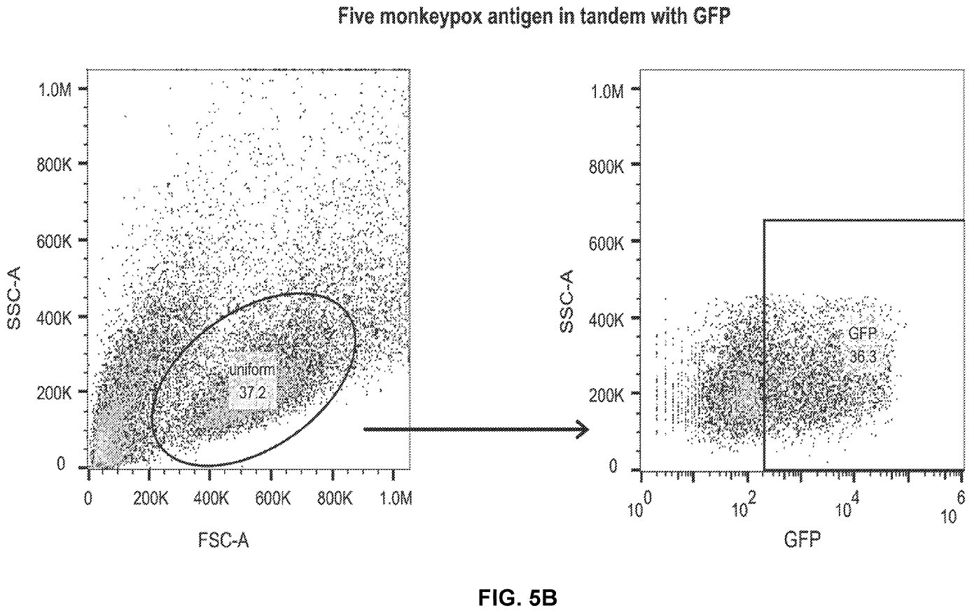

To test whether this multivalent vaccine construct encoding five monkeypox antigens in tandem could be successfully translated, a reporter construct otherwise identical to the vaccine was made in parallel by appending GFP to the end of tandem construct (MPXV-PentAg-GFP) (FIG. 1C). After transfection with this GFP reporter, around 36% of 293T cells expressed GFP which was quantified by flow cytometry (FIG. 5) and observed under fluorescence microscope. The expression of GFP represents the successful translation of all residues from same mRNA transcript in a significant population of cells. The MPXVac-097 mRNA expression construct was then developed into a lipid nanoparticle (LNP) formulation (Methods) and the transcribed MPXVac-097 mRNA was successfully encapsulated in LNP, of which size distribution was determined by dynamic light scattering. The encapsulated MPXVac-097 LNP mRNA was monodispersed with an average radius of 49 nm and polydispersity index of 0.16 (FIG. 1D).

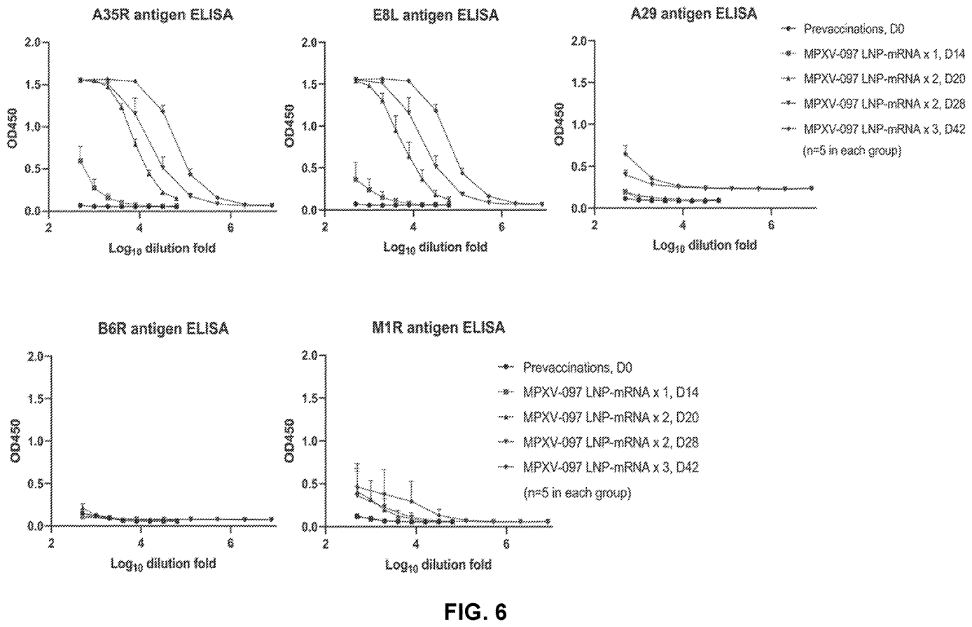

After confirming the successful translation of tandem construct and uniform size of the MPXVac-097 LNP-mRNA vaccine candidate, its initial immunogenicity in mice was then characterized. Mice were immunized with three doses of 8 μg MPXVac-097 LNP mRNA two weeks apart (prime, boost and booster). The retro-orbital blood was collected on day 0, 14, 28 (just before prime, boost, booster) and day 20, 42 (6 days post boost and 14 days post booster). The isolated plasma from blood was used to quantify antibody titers against individual monkeypox antigen in enzyme-linked immunosorbent assay (ELISA). Two weeks post prime on day 14, a modest and insignificant increase of antibody titers against A35R and E8L was found in half of vaccinated mice (FIGS. 1E and 6), suggesting a single dose (prime alone) is insufficient to elicit a strong antibody response. After the second and third doses, the antibody titer of A35R and E8L increased substantially, and became evident and significant in all mice on day 20, 28 and 42 (FIGS. 1E and 6). Interestingly, the antibody titer against M1R, that had no change post prime, started to increase moderately post boost and booster in one mouse (FIGS. 1E and 6). Because the M1R antigen was sandwiched between A35R and E8L on the same mRNA transcript, its expression level would be comparable to A35R and E8L. The delayed increase of M1R antibody titer in one mouse was unlikely due to differential expression of antigens and may stem from the difference of antigen immunogenicity or surface display. Minimal antibody response was detected for either A29L or B6R post boost, again highlighting the differences in immunogenicity between these antigens in the setting of mRNA vaccination.

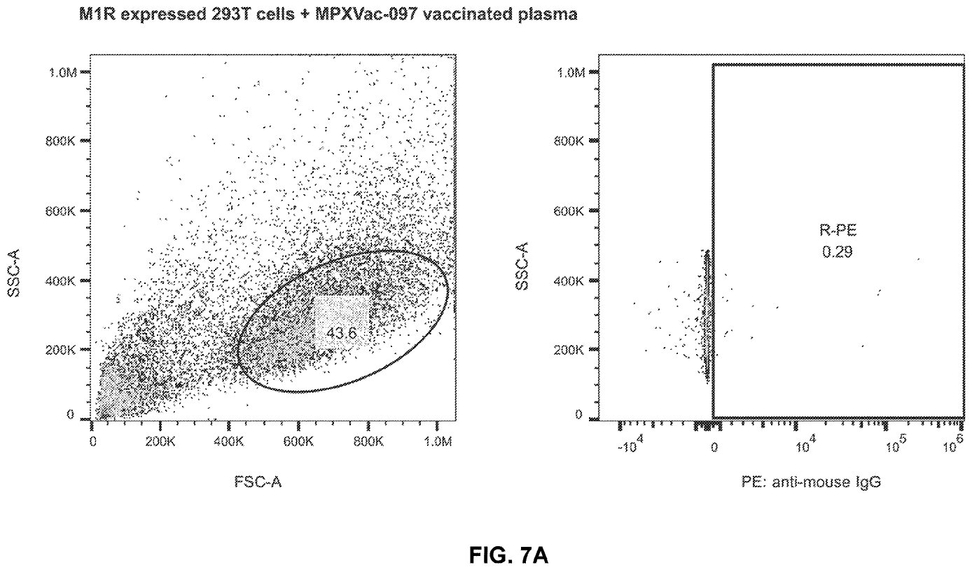

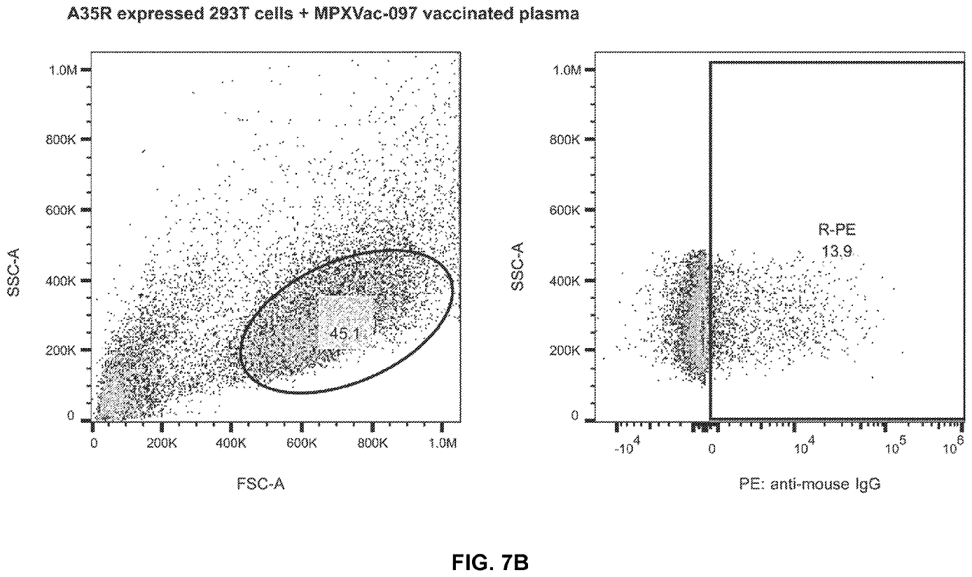

The antigens used in ELISA are purified ectodomains or truncating variants. Next, it was sought to ask if the full-length MPXV antigens can be presented on cell surface and recognized by the plasma antibodies elicited by MPXVac-097. The five monkeypox antigens were separately expressed in 293T cells from which surface antigens were detected by plasma antibody from mice on day 20 and anti-mouse IgG antibody with PE on flow cytometry (FIGS. 1F and 7). Expression of A35R and E8L led to an increase (12.6% and 1.2% respectively vs. 0.4% in negative control of SARS-COV-2 spike) of 293T cells that were bound to the MPXV plasma antibodies (FIGS. 1F and 7). This increase of MPXV antibody-bound cells was consistent with findings in ELISA, and validated the observation of antibody response to surface presentation of A35R and E8L cellular antigens.

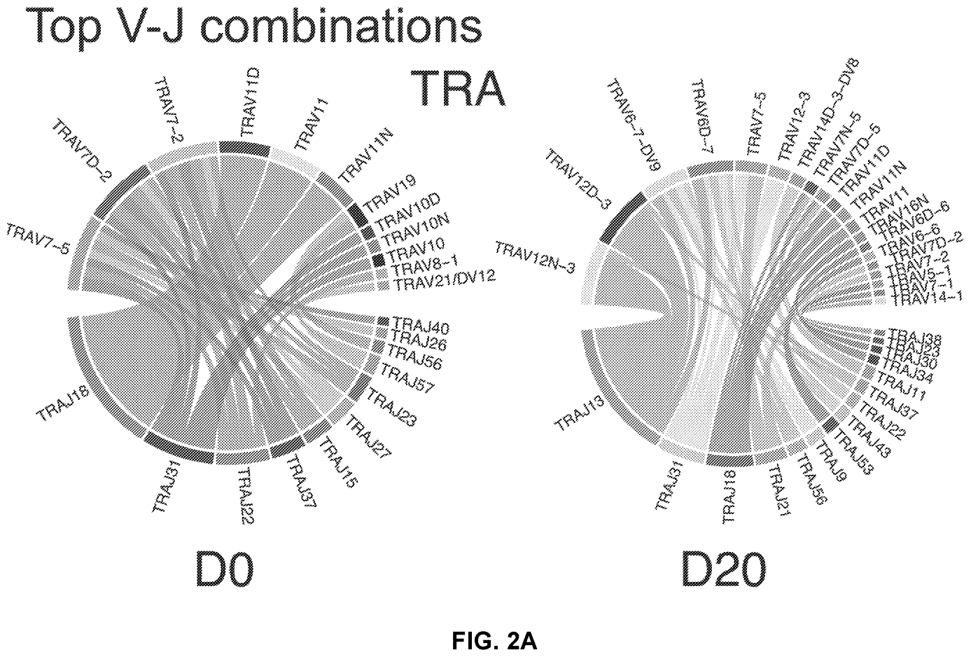

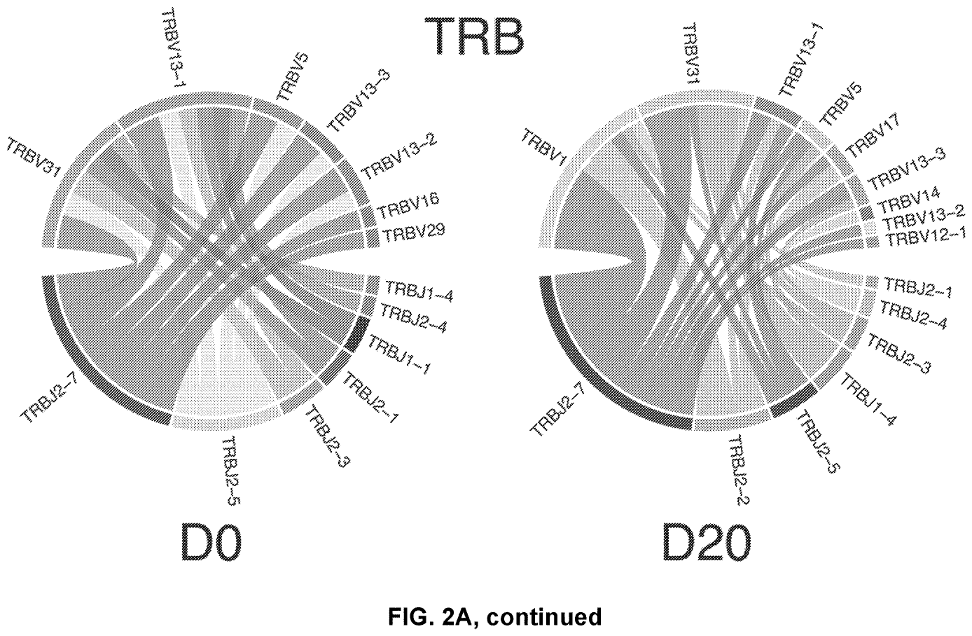

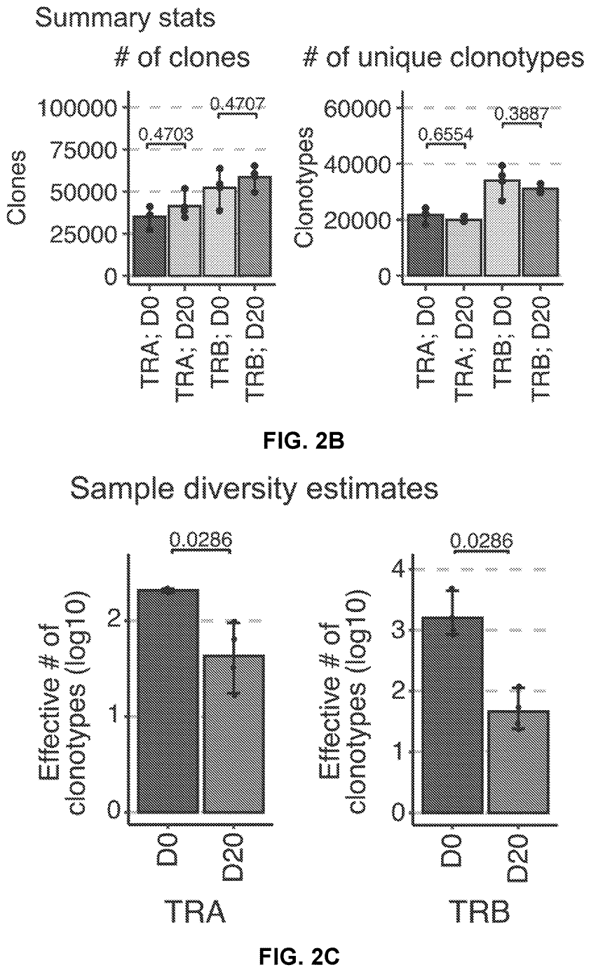

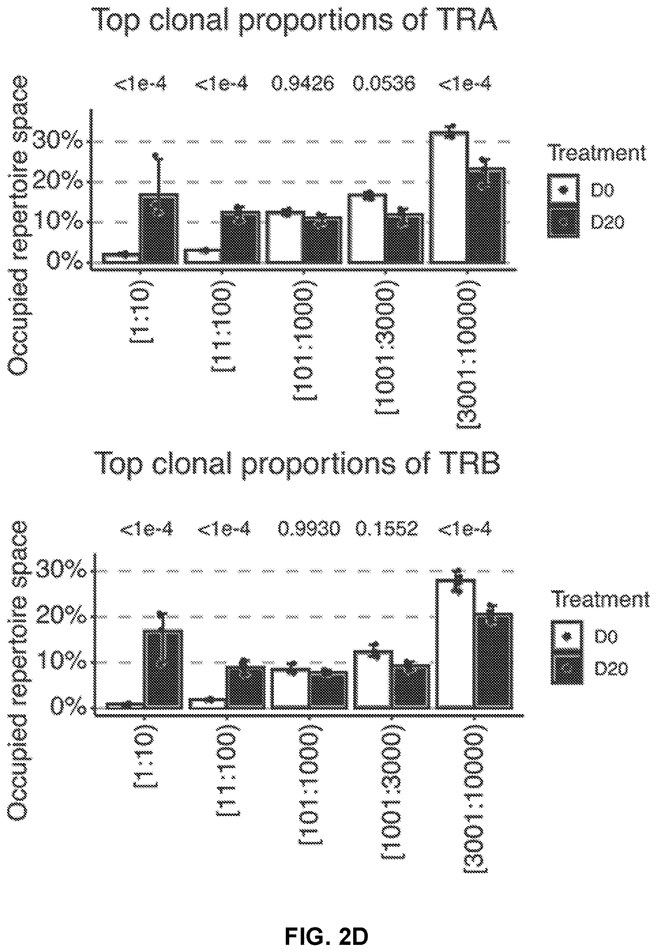

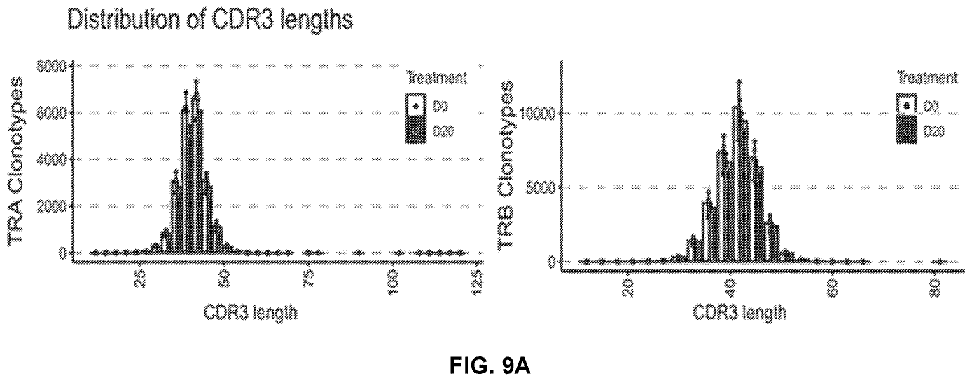

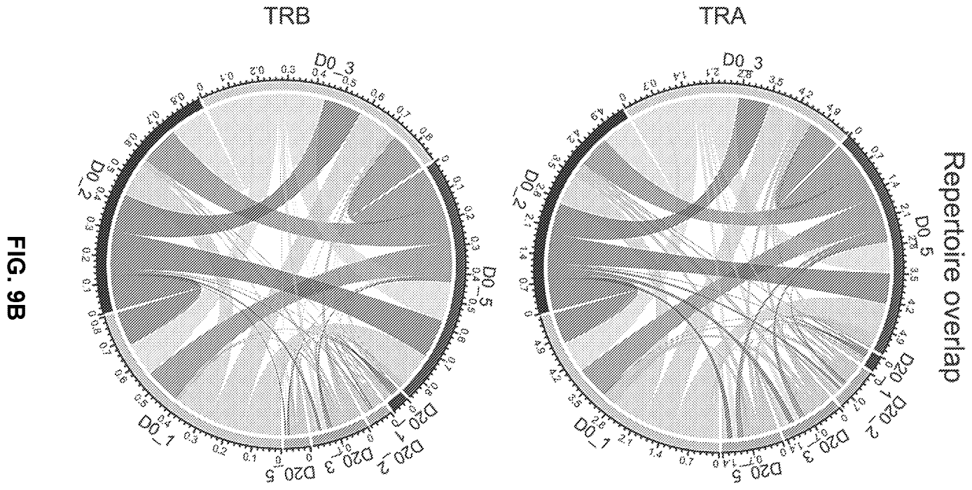

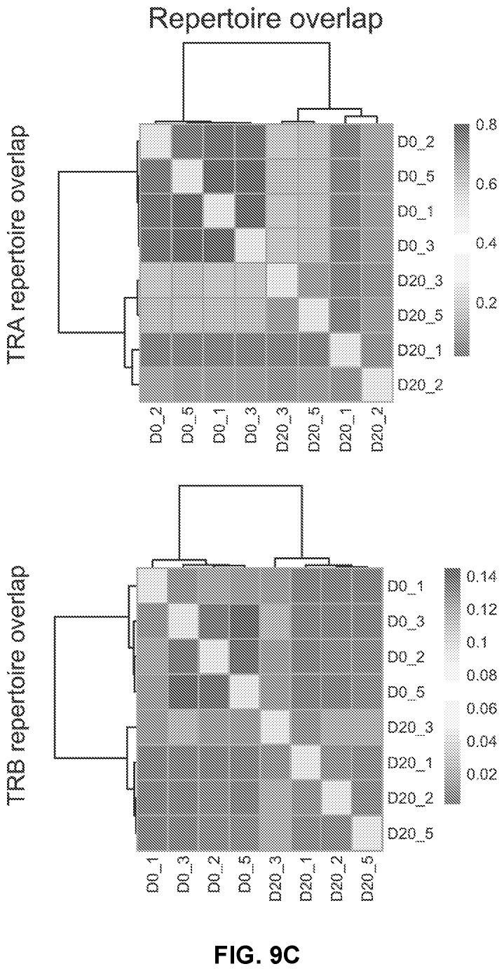

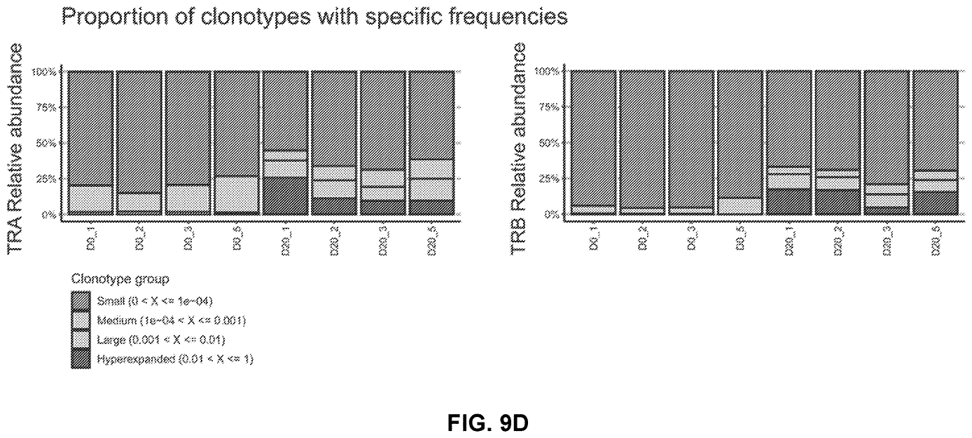

The ELISA and cell-surface antigen assays provided evidence of the initial B cell response to five monkeypox antigens in mice immunized with MPXVac-097. A natural question that follows is whether T cell response was induced in these animals. Subsequent studies then sought to characterize the T cell response by profiling the repertoire of T cell receptors (TCR) in vaccinated mice via bulk TCR-sequencing (TCR-seq). TCR-seq of peripheral blood mononuclear cells (PBMCs) revealed VJ combination usage and clonality maps of TCRs from mice before and after two MPXVac-097 LNP mRNA injections on day 0 and day 20, respectively (FIGS. 2 and 9). MPXVac-097 LNP mRNA vaccination resulted in more frequent use of TRAJ13, TRAV12N-3, TRAV12D-3 in alpha chain and TRBJ1-4, TRBJ2-2, TRBV1 in beta chain (FIG. 2A). After immunization, the most frequent VJ combinations was switched to TRAJ13-TRAV12N-3/TRAV12D-3 and TRBJ2-7-TRABV1. The total number of clones and unique clones were not significantly different between samples on day 0 and 20 (FIG. 2B). The CDR3 length distributions are also similar (FIG. 9A). In contrast, while the TCR repertoire compositions are similar between independent mice pre-vaccination, distinct TCR repertoire compositions between day 0 and day 20 groups was observed, suggesting that repertoire compositions changed substantially upon two-dose MPXVac-097 vaccination (FIGS. 9B-9C). Moreover, the clonal diversity of samples post boost was significantly reduced in two-dose MPXVac-097 vaccinated as compared to vaccine-naïve animals (FIG. 2C). There were also significant reduction of low-frequency clones and significant increase in expended or hyperexpanded clones (FIGS. 2D and 9D), which together are indicative of clonal expansion induced by MPXVac-097 vaccination.

In summary, and without wishing to be bound by theory, the studies of the current disclosure designed a multivalent monkeypox LNP-mRNA vaccine candidate, MPXVac-097, which can elicit significant antibody response against a subset of MPXV antigens (A35R and E8L) as well as T cell response in mice. Taken together, these data demonstrated initial feasibility of MPXV mRNA vaccine design and provided initial evidence of functionality for its future optimization.

### Example 2: Engineering mRNA Antigens for Enhanced Cell Surface Translocation and Immunogenicity

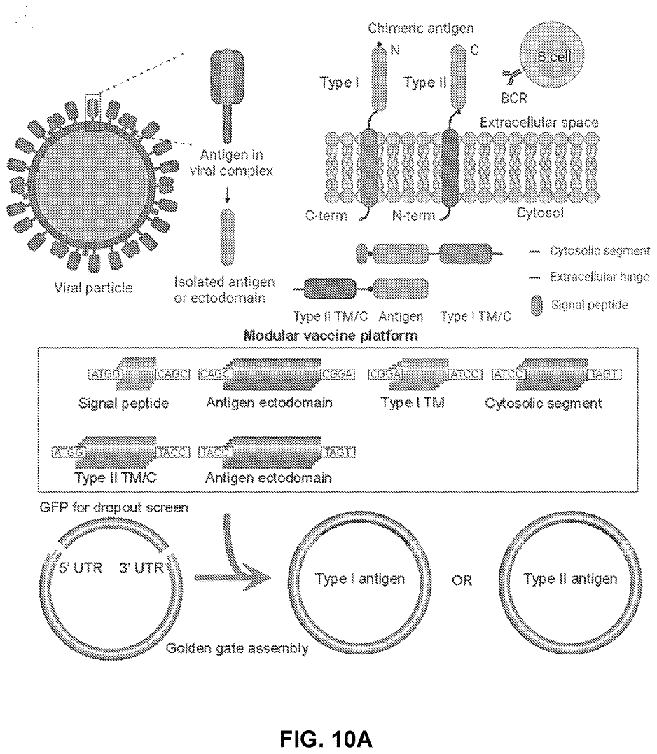

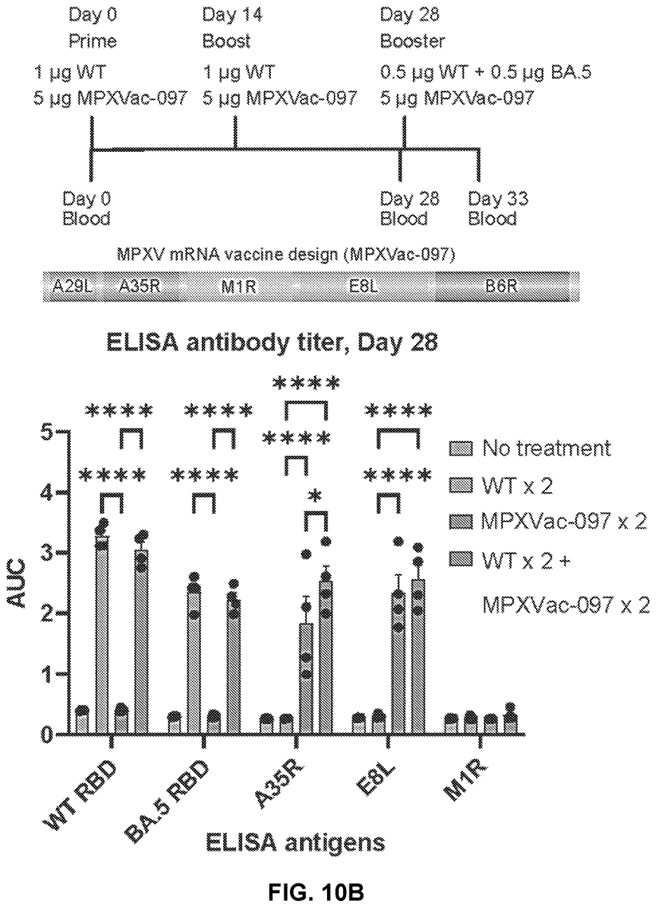

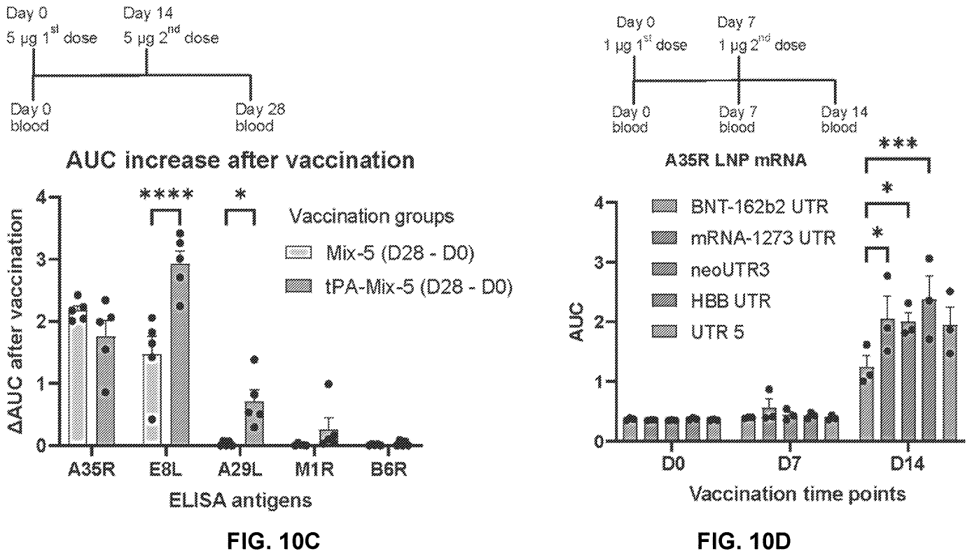

Having demonstrated the capability of the MPXVac-097 vaccine to elicit immune responses against a number of monkeypox antigens, a series of studies were then undertaken in order to evaluate strategies for further enhancing immunogenicity. FIG. 10A illustrates a schematic of the modified MPXVac-097 construct and the workflow of assembling the constructs. In these studies, two distinct approaches mediated by type I and II transmembrane domain and cytosolic segments (TM/Cs) were used to display cytosolic antigens, such as A29L on cell surface for B cell recognition. Some of the constructs were further modified with a signal peptide, which directs the translated construct into the secretory pathway and eventually to the cell surface. FIG. 10B, top illustrates a workflow of the animal studies disclosed herein which validated the various MPXVac-097-based constructs. FIG. 10B illustrates a direct comparison of the antibody responses elicited by SARS-COV-2 spike and MPXVac-097 mRNA antigens alone or in combination (n=4 on day 28). In a subsequent set of studies, various signal peptide sequences were used to enhance antibody responses. FIG. 10C demonstrates that the use of tPA signal peptide improved antibody response to E8L and A29L, but not A35R, M1R or B6R (n=4). A similar study also evaluated the effect of UTR pairs on the antibody response to A35R mRNA antigens (n=3), the results of which are illustrated in FIG. 10D.

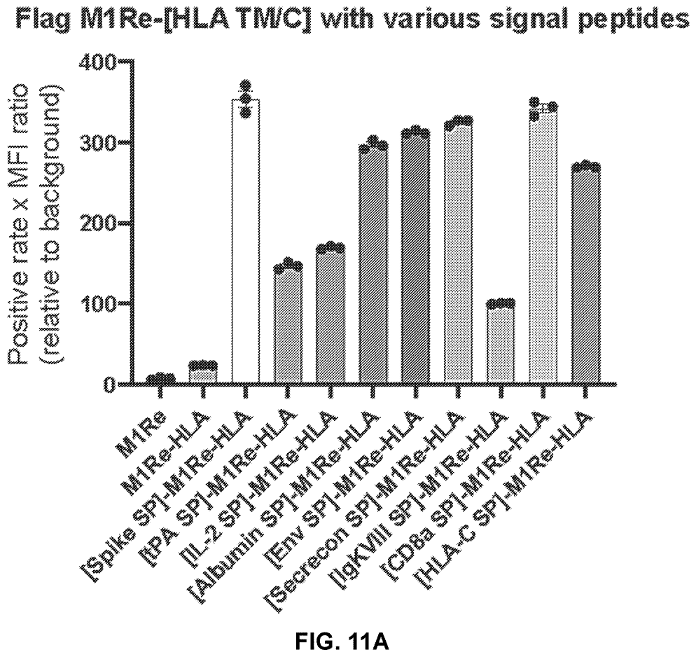

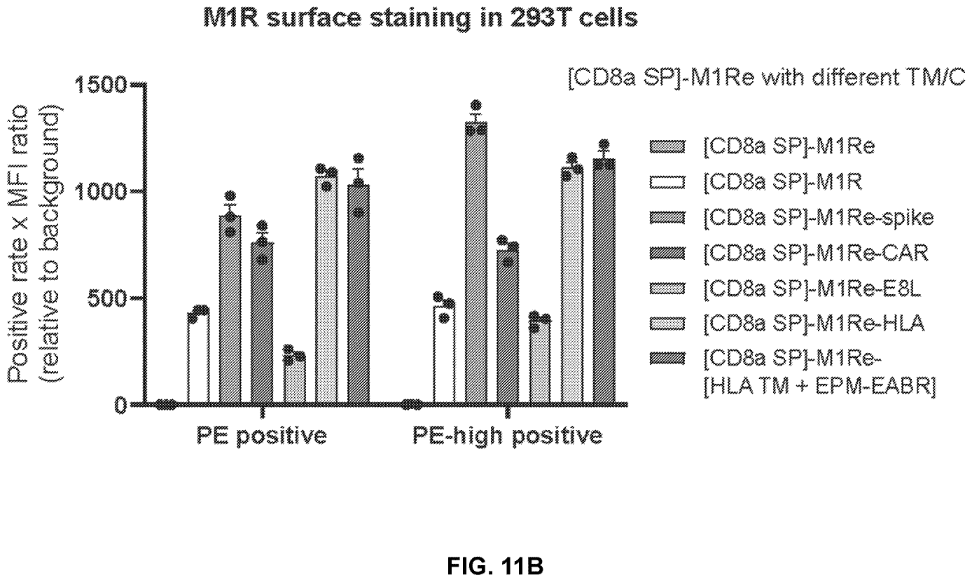

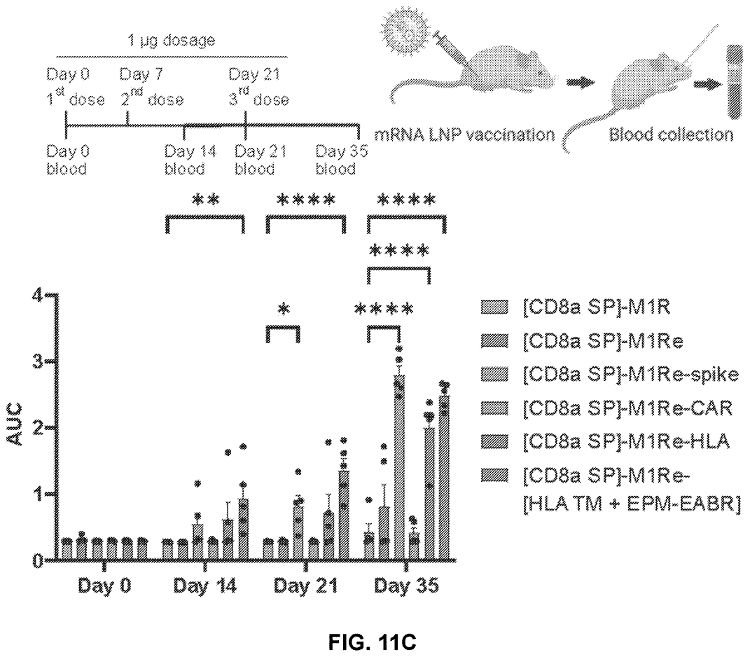

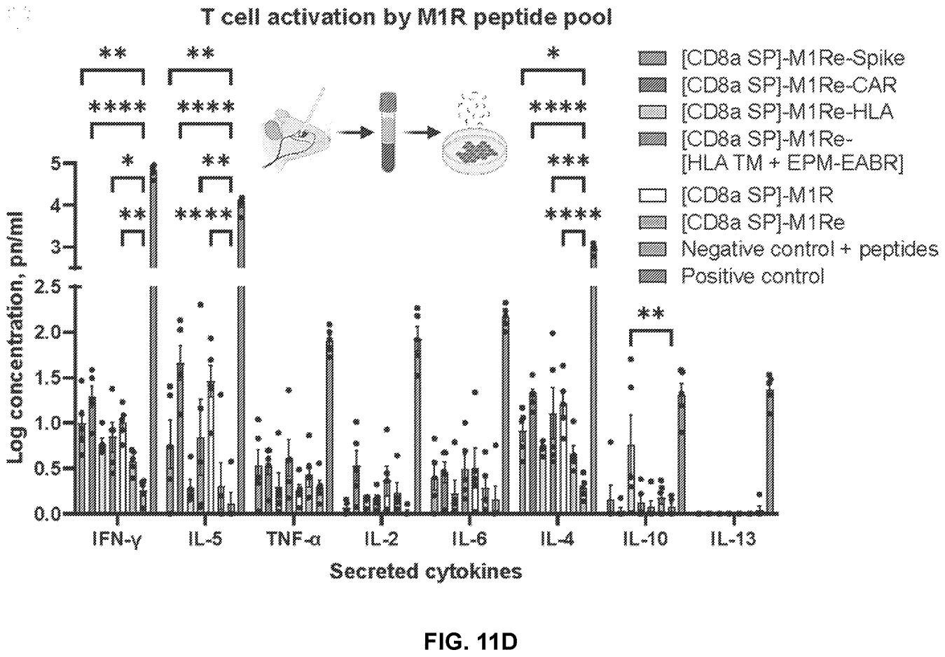

Studies then focused on improving the immunogenicity of the M1R antigen, which elicited significantly weaker response in previous studies. First, the effect of various different signal peptides (SP) was examined, which increased the cell-surface expression of M1R ectodomain (M1Re) fused to HLA TM/C (FIG. 11A). Here the greatest increase was observed using the CD8 signal peptide, which was selected for use in subsequent studies. Next, different type I TM/C improved were assessed, with the hope that increased cell-surface expression of [CD8a SP]-M1Re. Recombination of [CD8a SP]-M1Re with different type I TM/Cs significantly improved its surface expression in cells (n=3). The statistical significance was derived from comparisons of each group with [CD8a SP]-M1R control with M1R native TM/C (FIG. 11B). FIG. 11C, here heterologous TM/Cs fused to [CD8a SP]-M1Re significantly improved antibody response to M1R (n=5). FIG. 11D illustrates another study in which the M1R-specific T cell response was modulated via the use of heterologous TM/Cs. PBMCs collected from mice on day 35 after three LNP mRNA vaccinations with indicated chimeric M1R antigens were stimulated by M1R peptides and secreted cytokines as indicators of MPXV-specific T cell response were quantified (n=5).

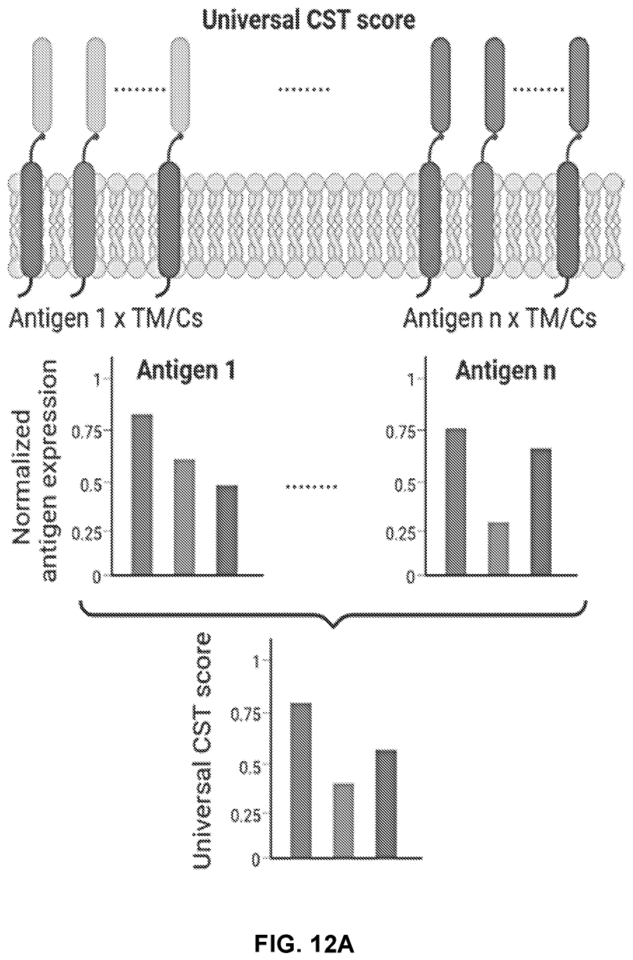

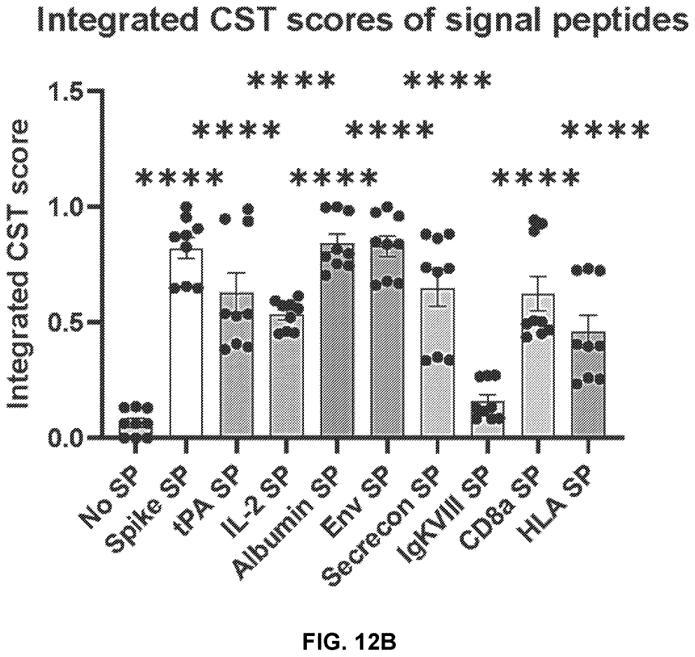

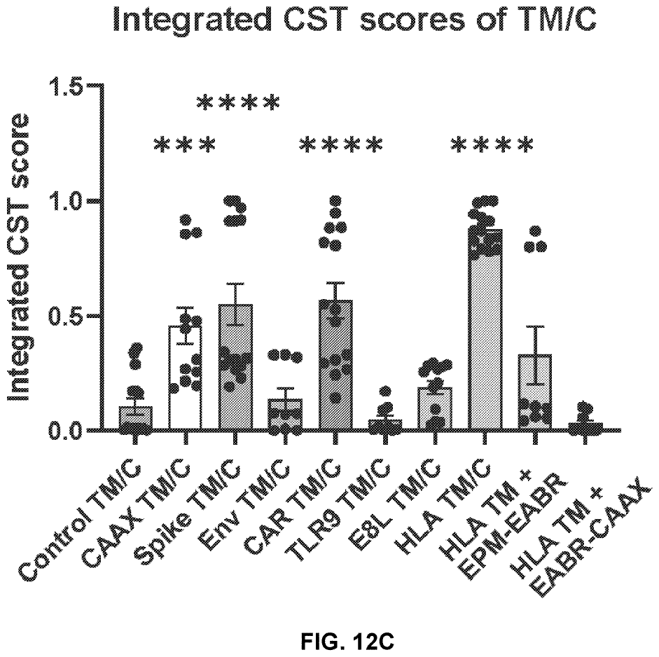

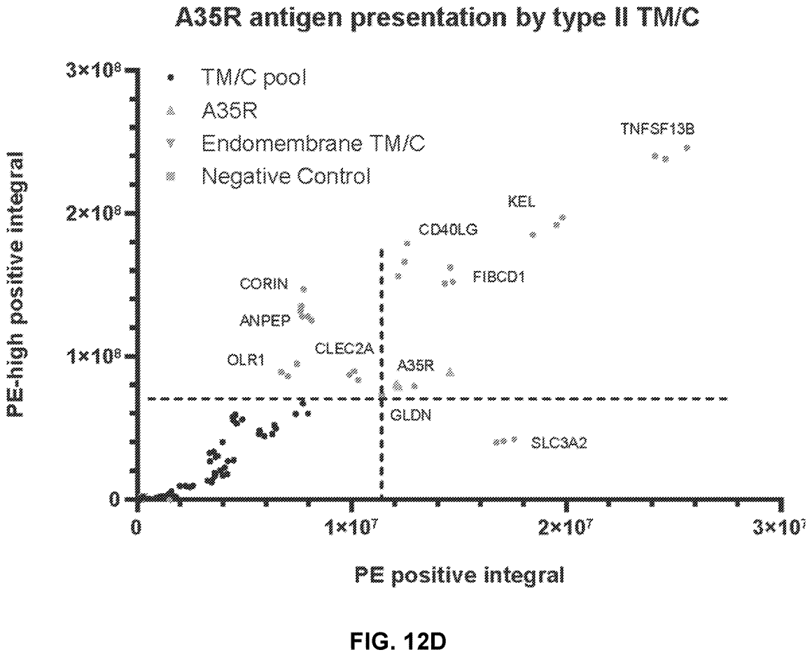

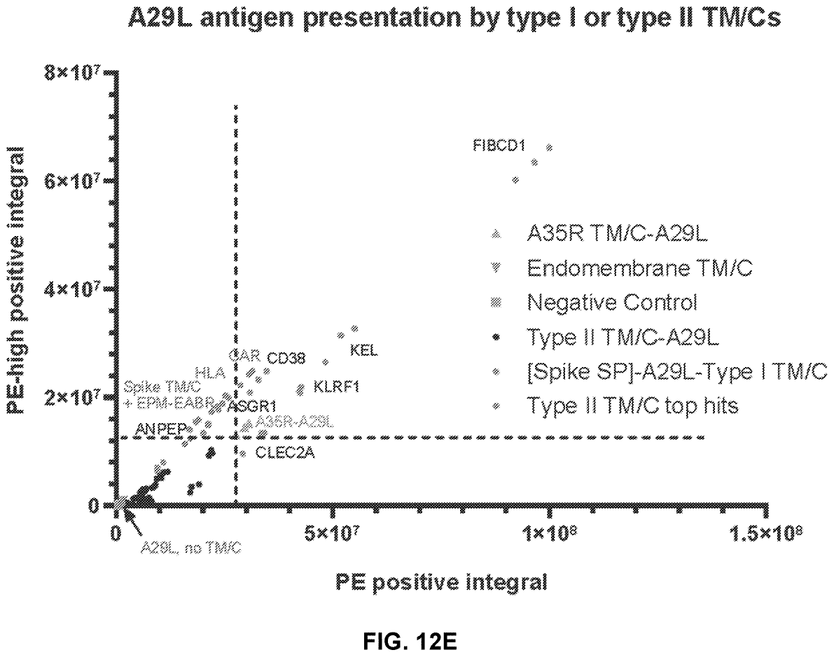

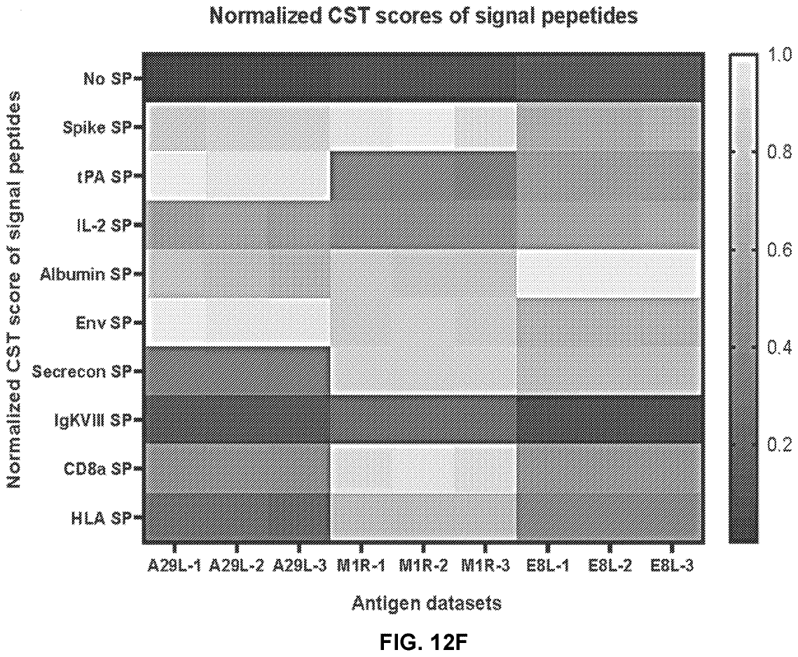

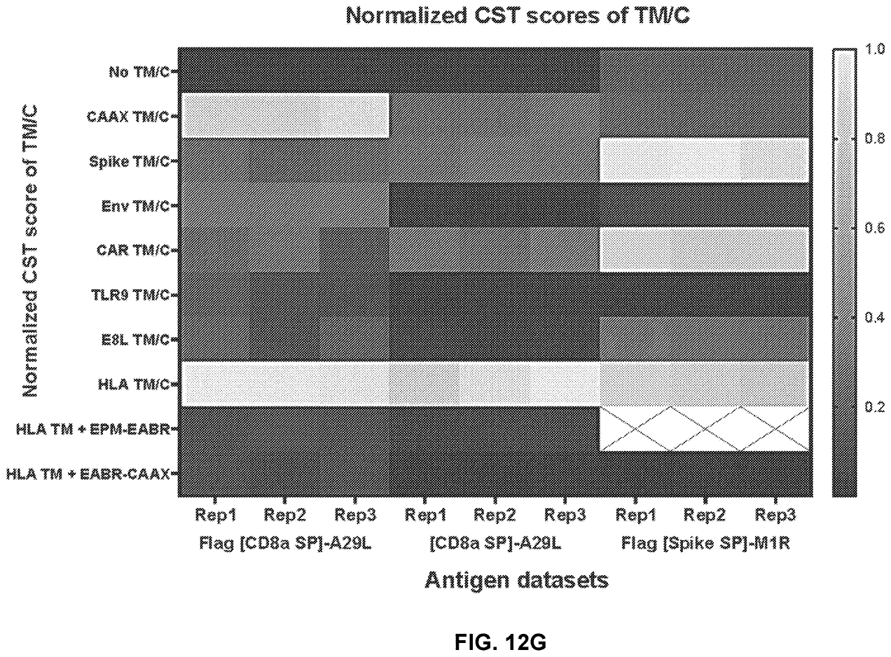

Lastly, studies were conducted in which CST scores were determined for each vaccine variant in order to quantify the effectiveness of each signal peptide and TM/C at increasing immunogenicity. FIG. 12A is a schematic that shows the calculation of universal CST score using multiple antigen datasets. In FIGS. 12B-12C, universal CST scores of type I SP (FIG. 12B) and TM/C (FIG. 12C) were determined. For FIG. 12D, A subset of type II TM/Cs was determined to result in higher A35R ectodomain surface expression than native A35R. In FIG. 12E, the surface expression of A29L, which was mediated by type I or type II TM/Cs was quantified. Here, the untagged antigen was stained using anti-A35R or anti-A29L antibody (n=3).

TABLE 1
MPXV antigen sequences

| SEQ | | |
|---|---|---|
| ID | | |
| NO: | Name | Sequence |
| 1. | MPXV- | MPQQLSPINIETKKAISDARLKTLDIHYNESKPTTIQNTGKLVRINFKGGYISGG |
| | ON563414. | FLPNEYVLSTHIHIYWGKEDDYGSNHLIDVYKYSGEINLVHWNKKKYSSYEEAKK |
| | 3E8L | HDDGIIIIAIFLQVSDHKNVYFQKIVNQLDSIRSANMSAPFDSVFYLDNLLPSTL |
| | | DYFTYLGTTINHSADAAWIIFPTPINIHSDQLSKERTLLSSSNHEGKPHYITENY |
| | | RNPYKLNDDTQVYYSGEIIRAATTSPVRENYFMKWLSDLREACFSYYQKYIEGNK |
| | | TFAIIAIVFVFILTAILFLMSQRYSREKQN |
| 2. | MPXV- | MPQQLSPINIETKKAISNARLKPLDIHYNESKPTTIQNTGKLVRINEKGGYISGG |
| | AY603355. | FLPNEYVLSTSLRIYWGKEDDYGSNHLIDVYKYSGEINLVHWNKKKYSSYEEAKK |
| | 1E8L | HDDGLIIISIFLQVSDHKNVYFQKIVNQLDSIRSTNTSAPFDSVFYLDNLLPSKL |
| | | DYFTYLGTTINHSADAVWIIFPTPINIHSDQLSKERTLLSSSNHDGKPHYITENY |
| | | RNPYKLNDDTQVYYSGEIIRAATTSPARENYFMRWLSDLRETCESYYQKYIEGNK |
| | | TFAIIAIVFVFILTAILFFMSQRYSREKQN |
| 3. | MPXV- | MGAAASIQTTVNTLSERISSKLEQEANASAQTKCDIEIGNFYIRQNHGCNITVKN |
| | ON563414. | MCSADADAQLDAVLSAATETYSGLTPEQKAYVPAMFTAALNIQTSVNTVVRDFEN |
| | 3MIR | YVKQTCNSSAVVDNKLKIQNVIIDECYGAPGSPTNLEFINTGSSKGNCAIKALMQ |
| | | LTTKATTQIAPRQVAGTGVQFYMIVIGVIILAALFMYYAKRMLFTSTNDKIKLIL |
| | | ANKENVHWTTYMDTFFRTSPMIIATTDIQN |
| 4. | MVA- | MGAAASIQTTVNTLSERISSKLEQEANASAQTKCDIEIGNFYIRQNHGCNLTVKN |
| | AY603355. | MCSADADAQLDAVLSAATETYSGLTPEQKAYVPAMFTAALNIQTSVNTVVRDFEN |
| | 1MIR | YVKQTCNSSAVVDNKLKIQNVIIDECYGAPGSPTNLEFINTGSSKGNCAIKALMQ |
| | | LTTKATTQIAPRQVAGTGVQFYMIVIGVIILAALFMYYAKRMLFTSTNDKIKLIL |
| | | ANKENVHWTTYMDTFFRTSPMVIATTDMQN |
| 5. | MPXV- | MKTISVVTLLCVLPAVVYSTCTVPTMNNAKLTSTETSFNDKQKVTFTCDSGYHSL |
| | ON563414. | DPNAVCETDKWKYENPCKKMCTVSDYVSELYDKPLYEVNSTMTLSCNGETKYFRC |
| | 3B6R | EEKNGNTSWNDTVTCPNAECQPLOLEHGSCQPVKEKYSFGEYMTINCDVGYEVIG |
| | | VSYISCTANSWNVIPSCQQKCDIPSLSNGLISGSTFSIGGVIHLSCKSGFTLTGS |
| | | PSSTCIDGKWNPILPTCVRSNEEFDPVDDGPDDETDLSKLSKDVVOYEQEIESLE |
| | | ATYHIIIMALTIMGVIFLISIIVLVCSCDKNNDQYKFHKLLP |
| 6. | MVA- | MKTISVVTLLCVLPAVVYSTCTVPTMNNAKLTSTETSENNNQKVTFTCDOGYHSS |
| | AY603355. | DPNAVCETDKWKYENPCKKMCTVSDYISELYNKPLYEVNSTMTLSCNGETKYFRC |
| | 1B6R | EEKNGNTSWNDTVTCPNAECQPLQLEHGSCQPVKEKYSFGEYITINCDVGYEVIG |
| | | ASYISCTANSWNVIPSCQQKCDIPSLSNGLISGSTFSIGGVIHLSCKSGFILTGS |
| | | PSSTCIDGKWNPILPTCVRSNEKFDPVDDGPDDETDLSKLSKDVVQYEQEIESLE |
| | | ATYHIIIVALTIMGVIFLISVIVLVCSCDKNNDQYKFHKLLP |
| 7. | MPXV- | MMTPENDEEQTSVFSATVYGDKIQGKNKRKRVIGLCIRISMVISLLSMITMSAFL |
| | ON563414. | IVRLNQCMSANKAAITDSAVAVAAASSTHRKVVSSTTQYDHKESCNGLYYQGSCY |
| | 3A35R | ILHSDYKSFEDAKANCAAESSTLPNKSDVLTTWLIDYVEDTWGSDGNPITKTTSD |
| | | YQDSDVSQEVRKYFCT |
| 8. | MVA- | MMTPENDEEQTSVFSATVYRDKIQGKNKRKRVIGLCIRISMVISLLSMITMSAFL |
| | AY603355. | IVRLNQCMSANEAAITDAAVAVAAASSTHRKVASSTTQYDHKESCGLYYQGSCYI |
| | 1A35R | LHSDYQLESDAKANCTAESSTLPNKSDVLTTWLIDYVKDTWGSDGNPITKTTSDY |
| | | QDSDVSQEVRKYFCVKTMN |
| 9. | MPXV- | MDGTLFPGDDDLAIPATEFESTKAAKNPETKREAIVKAYGDDNEETLKQRLTNLE |
| | ON563414. | KKITNITTKFEQIEKCCKRNDEVLERLENHAETLRAAMISLAKKIDVQTGRHPYE |
| | 3A29L | |
| 10. | MVA- | MDGTLFPGDDDLAIPATEFFSTTAAKKPEAKREAIVKADEDDNEETLKQRLTNLE |
| | AY603355. | KKITNVTTKFEQIEKCCKRNDEVLERLENHAETLRAAMISLAKKIDVQTGRRPYE |
| | 1A29L | |

TABLE 2
Sequences of Vaccine Constructs, and TM/C and SP domains

| SEQ | | |
|---|---|---|
| ID | | |
| NO: | Name: | Sequence: |
| 11. | HLA- | PSSQPTIPIMGIVAGLAVLVVLAVLGAVVTAMMCGSGKKKKKKQKTKCVIM |
| | CAAXTM/ | |
| | C Domain | |
| 12. | Spike | SPDVDLGDISGINASVVNIQKEIDRLNEVAKNLNESLIDLQELGKYEQYIKW |
| | TM/C | PWYIWLGFIAGLIAIVMVTIMLGSCCMTSCCSCLKGCCSCGSCCKEDEDDSE |
| | Domain | PVLKGVKLHYT |
| 13. | Env | DKWASLWNWFNITNWLWYIKLFIMIIGGLVGLRTVCAVLSIVGSNRVROGYS |
| | TM/C | PLSFQTRLPNPRGPGRPEETEGEGGERDRDRS |
| | Domain | |
| 14. | CARTM/ | TTTPAPRPPTPAPTIASQPLSLRPEACRPAAGGAVHTRGLDFACDIYIWAPL |
| | C Domain | AGTCGVLLLSLVITLYCGSKRGRKKLLYIFKOPFMRPVQTTQEEDGCSCREP |
| | | EEEEGGCELRVKFSRSADAPAYQQGQNQLYNELNLGRREEYDVLDKRRGRDP |
| | | EMGGKPRRKNPQEGLYNELQKDKMAEAYSEIGMKGERRRGKGHDGLYQGLST |
| | | ATKDTYDALHMQALPPR |
| 15. | CD86- | QEFPSPQTYWKEITASVTVALLLVMLLIIVCHGSKKPNQPSRPSNTASKLER |
| | TLR9TM/ | DSNADRETINLKELEPQIASAKPNAECGWDVWYCFHLCLAWLPLLARSRRSA |
| | C Domain | QTLPYDAFVVEDKAQSAVADWVYNELRVRLEERRGRRALRLCLEDRDWLPGQ |
| | | TLFENLWASIYGSRKTLFVLAHTDRVSGLLRTSFLLAQQRLLEDRKDVVVLV |
| | | ILRPDAHRSRYVRLRQRLCRQSVLFWPQQPNGQGGFWAQLSTALTRDNRHFY |
| | | NQNFCRGPTAE |
| 16. | E8LTM/ | SGEIIRAATTSPVRENYFMKWLSDLREACFSYYQKYIEGNKTFAIIAIVFVFI |
| | C Domain | LTAILFLMGSSQRYSREKON |
| 17. | HLATM/ | PSSQPTIPIMGIVAGLAVLVVLAVLGAVVTAMMCGSRRKSSGGKGGSCSQAAC |
| | C Domain | SNSAQGSDESLITCKA |
| 18. | HLA- | PSSQPTIPIMGIVAGLAVLVVLAVLGAVVTAMMCGSALPGNPDHREMGETLPE |
| | EPM- | EVGEYRQPSGGSVPVSPGPPSGLEPTSSSPYGGGSENSSINNIHEMEIQLKDA |
| | EABRTM/ | LEKNQQWLVYDQQREVYVKGLLAKIFELEKKTETAAHSLP |
| | C Domain | |
| 19. | HLA- | PSSQPTIPIMGIVAGLAVLVVLAVLGAVVTAMMCGSGGGSENSSINNIHEMEI |
| | EABR- | QLKDALEKNQQWLVYDQQREVYVKGLLAKIFELEKKTETAAHSLPGGGSGKKK |
| | CAAXTM/ | KKKQKTKCVIM |
| | C Domain | |
| 20. | CD8 | MALPVTALLLPLALLLHAARP |
| | Signal | |
| | Peptide | |
| 21. | SARS- | MFVFLVLLPLVSS |
| | CoV-2 | |
| | SP | |
| 22. | tPA | MDAMKRGLCCVLLLCGAVFVSPS |
| | Signal | |
| | Peptide | |
| 23. | IL-2 | MYRMQLLSCIALSLALVINS |
| | Signal | |
| | Peptide | |
| 24. | Albumin | MKWVTFISLLFLESSAYS |
| | Signal | |
| | Peptide | |
| 25. | Env | MKVMGTKKNYQHLWRWGIMLLGMLMMSSAA |
| | Signal | |
| | Peptide | |
| 26. | Secrecon | MWWRLWWLLLLLLLLWPMVWA |
| | Signal | |
| | Peptide | |
| 27. | IgKVIII | MDMRVPAQLLGLLLLWLRGARC |
| | Signal | |
| | Peptide | |
| 28. | KELTM/ | MEGGDQSEEEPRERSQAGGMGTLWSQESTPEERLPVEGSRPWAVARRVLTAIL |
| | C Domain | ILGLLLCFSVLLFYNFQNCGPRPCET |
| 29. | FIBCD1TM/ | MVNDRWKTMGGAAQLEDRPRDKPQRPSCGYVLCTVLLALAVLLAVAVTGAVLF |
| | C Domain | LNHAHAPGTAPPP |
| 30. | ANPEPTM/ | AKGFYISKSLGILGILLGVAAVCTIIALSVVYSQEKNKNANSSPVASTTPSAS |
| 31. | ASGR1TM/ | MTKEYQDLQHLDNEESDHHQLRKGPPPPQPLLORLCSGPRLLLLSLGLSLLLL |
| | C Domain | VVVCVIGSQNS |
| 32. | CD38TM/ | MANCEFSPVSGDKPCCRLSRRAQLCLGVSILVLILVVVLAVVVPRWRQQWSGP |
| | C Domain | GTT |
| 33. | CD40LGTM/ | MIETYNQTSPRSAATGLPISMKIFMYLLTVFLITQMIGSALFAVYLHRRLDKI |
| | C Domain | EDERNLHE |
| 34. | CLEC2ATM/ | MINPELRDGRADGFIHRIVPKLIQNWKIGLMCFLSIIITTVCIIMIATWSKHA |
| | C Domain | KPVACSGDW |
| 35. | CORINTM/ | MKOSPALAPEERCRRAGSPKPVLRADDNNMGNGCSQKLATANLLRELLLVLIP |
| | C Domain | CICALVLLLVILLSYVGTLQK |
| 36. | GLDNTM/ | MARGAEGGRGDAGWGLRGALAAVALLSALNAAGTVFALCQWRGLSSALRA |
| | C Domain | |
| 37. | MPXVac- | MDGTLFPGDDDLAIPATEFESTKAAKNPETKREAIVKAYGDDNEETLKORLT |
| | 097 | NLEKKITNITTKFEQIEKCCKRNDEVLERLENHAETLRAAMISLAKKIDVQT |
| | | GRHPYESGSGEGRGSLLTCGDVEENPGPTSKLMMTPENDEEQTSVESATVYG |
| | | DKIQGKNKRKRVIGLCIRISMVISLLSMITMSAFLIVRLNQCMSANKAAITD |
| | | SAVAVAAASSTHRKVVSSTTQYDHKESCNGLYYQGSCYILHSDYKSFEDAKA |
| | | NCAAESSTLPNKSDVLTTWLIDYVEDTWGSDGNPITKTTSDYQDSDVSQEVR |
| | | KYFCTSGSGATNFSLLKQAGDVEENPGPSRMGAAASIQTTVNTLSERISSKL |
| | | EQEANASAQTKCDIEIGNFYIRQNHGCNITVKNMCSADADAQLDAVLSAATE |
| | | TYSGLTPEQKAYVPAMFTAALNIQTSVNTVVRDFENYVKQTCNSSAVVDNKL |
| | | KIQNVIIDECYGAPGSPTNLEFINTGSSKGNCAIKALMQLTTKATTQIAPRQ |
| | | VAGTGVQFYMIVIGVIILAALFMYYAKRMLFTSTNDKIKLILANKENVHWTT |
| | | YMDTFFRTSPMIIATTDIQNSGSGQCTNYALLKLAGDVESNPGPSGMPQQLS |
| | | PINIETKKAISDARLKTLDIHYNESKPTTIQNTGKLVRINFKGGYISGGFLP |
| | | NEYVLSTIHIYWGKEDDYGSNHLIDVYKYSGEINLVHWNKKKYSSYEEAKKH |
| | | DDGIIIIAIFLQVSDHKNVYFQKIVNOLDSIRSANMSAPFDSVFYLDNLLPS |
| | | TLDYFTYLGTTINHSADAAWIIFPTPINIHSDQLSKERTLLSSSNHEGKPHY |
| | | ITENYRNPYKLNDDTQVYYSGEIIRAATTSPVRENYFMKWLSDLREACFSYY |
| | | QKYIEGNKTFAIIAIVFVFILTAILFLMSQRYSREKONSGSGATNESLLKQA |
| | | GDVEENPGPPRMKTISVVTLLCVLPAVVYSTCTVPTMNNAKLTSTETSENDK |
| | | QKVTFTCDSGYHSLDPNAVCETDKWKYENPCKKMCTVSDYVSELYDKPLYEV |
| | | NSTMTLSCNGETKYFRCEEKNGNTSWNDTVTCPNAECQPLQLEHGSCQPVKE |
| | | KYSFGEYMTINCDVGYEVIGVSYISCTANSWNVIPSCQQKCDIPSLSNGLIS |
| | | GSTFSIGGVIHLSCKSGFTLTGSPSSTCIDGKWNPILPTCVRSNEEFDPVDD |
| | | GPDDETDLSKLSKDVVQYEQEIESLEATYHIIIMALTIMGVIFLISIIVLVC |
| | | SCDKNNDQYKFHKLLP |

### Enumerated Embodiments

The following enumerated embodiments are provided, the numbering of which is not to be construed as designating levels of importance.
Embodiment 1 provides an isolated messenger ribonucleic acid (mRNA) comprising a 5′ untranslated region (UTR), a 3′ UTR, and an open reading frame encoding at least one monkeypox virus antigen, wherein the protein sequence of the at least one monkeypox virus antigen comprises all or a portion of the protein sequence of a monkeypox envelope or cell surface binding protein.
Embodiment 2 provides the isolated mRNA of embodiment 1, wherein the at least one monkeypox virus antigen is selected from the group consisting of A29, E8L, M1R, A35R, B6R and any combination thereof.
Embodiment 3 provides the isolated mRNA of any one of embodiments 1-2, wherein the amino acid sequences encoding two or more monkeypox antigens are linked by independently selected linker sequences.
Embodiment 4 provides the isolated mRNA of embodiment 3, wherein the linker sequences comprise a 2A peptide sequence.
Embodiment 5 provides the isolated mRNA of any one of embodiments 1-4, further comprising one or more transmembrane domain and cytosolic segments (TM/Cs).
Embodiment 6 provides the isolated mRNA of embodiment 5, wherein the TM/C segment is selected from the group consisting of a HLA-CAAX, a Spike, an Env, a CAR, a CD86-TLR9, an E8L, an HLA, an HLA-EPM-EABR, an HLA-EABR-CAAX, a KEL, a FIBCD1, an ANPEP, an ASGR1, a CD38, a CD40LG, a CLEC2A, a CORIN, and a GLDN TM/C domain.
Embodiment 7 provides the isolated mRNA of embodiment 5, wherein the TM/C segment comprises an amino acid sequence selected from the group consisting of SEQ ID NOs: 11-19 and 28-36.
Embodiment 8 provides the isolated mRNA of embodiment 5, wherein the TM/C segment comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 11-19 and 28-36.
Embodiment 9 provides the isolated mRNA of any one of embodiments 1-8, further comprising a signal peptide.
Embodiment 10 provides the isolated mRNA of embodiment 9, wherein the signal peptide is selected from the group consisting of a CD8, a SARS COV2, a tPA, an IL2, an albumin, an Env, a Secrecon, and a IgKvIII signal peptide.
Embodiment 11 provides the isolated mRNA of embodiment 9, wherein the signal peptide is a CD8 signal peptide.
Embodiment 12 provides the isolated mRNA of embodiment 9, wherein the signal peptide comprises an amino acid sequence selected from the group consisting of SEQ ID NOs: 20-29.
Embodiment 13 provides the isolated mRNA of embodiment 9, wherein the signal peptide comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 20-29.
Embodiment 14 provides the isolated mRNA of any one of embodiments 1-13, wherein the isolated mRNA further comprises a reporter gene.
Embodiment 15 provides the isolated mRNA of embodiment 14, wherein the reporter gene is GFP or a variant thereof.
Embodiment 16 provides the isolated mRNA of any one of embodiments 1-15, wherein the at least one antigen protein sequence comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 1-10.
Embodiment 17 provides the isolated mRNA of any one of embodiments 1-16, wherein the at least one antigen protein sequence comprises an amino acid sequence set forth in SEQ ID NOs: 1-10.
Embodiment 18 provides the isolated mRNA of any one of embodiments 1-17, wherein the mRNA encodes an amino acid sequence set forth in SEQ ID NO: 37.
Embodiment 19 provides the isolated mRNA of any one of embodiments 1-17, wherein the mRNA encodes an amino acid sequence having 75%, 80%, 85%, 90%, 91%, 92%, 93%, 94%, 95%, 96%, 97%, 98%, 99%, 99.5% or more sequence identity to SEQ ID NO: 37.
Embodiment 20 provides an isolated polynucleotide encoding the mRNA of any one of embodiments 1-19.
Embodiment 21 provides the isolated polynucleotide of embodiment 20, wherein the polynucleotide comprises one or more promoters and/or a polyadenylation signal operably linked to a sequence encoding the mRNA.
Embodiment 22 provides a vector comprising the polynucleotide of any one of embodiments 20-21.
Embodiment 23 provides a method of producing a recombinant monkeypox antigen protein, the method comprising introducing the polynucleotide of any one of embodiments 20-21 and/or vector of embodiment 22 into a host cell and incubating the host cell under conditions sufficient for expression of the polynucleotide encoding the mRNA of any one of embodiments 1-19, thereby producing the monkeypox antigen protein.
Embodiment 24 provides a lipid nanoparticle comprising the mRNA of any one of embodiments 1-19.
Embodiment 25 provides the lipid nanoparticle of embodiment 24, wherein the molar ratio of lipid to mRNA is in the range of about 5:1 to 20:1, preferably 6:1.
Embodiment 26 provides the lipid nanoparticle of any one of embodiments 24-25, wherein the lipid nanoparticle comprises at least one ionizable cationic lipid, at least one helper lipid, at least one sterol, and/or at least one PEG-modified lipid.
Embodiment 27 provides the lipid nanoparticle of embodiment 26, wherein the at least one ionizable cationic lipid comprises 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), PNI ionizable lipid, SM-102, DLin-MC3-DMA, DLin-KC2-DMA, ALC-0315, or a combination thereof.
Embodiment 28 provides the lipid nanoparticle of embodiment 26, wherein the at least one helper lipid comprises 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC), 1,2-dioleoyl-sn-glycero-3-phosphocholine (DOPC), or a combination thereof.
Embodiment 29 provides the lipid nanoparticle of embodiment 26, wherein the at least one PEG-modified lipid comprises 1,2-dimyristoyl-racglycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, mPEG-DPPE, or a combination thereof.
Embodiment 30 provides the lipid nanoparticle of embodiment 26, wherein the at least one sterol is cholesterol.
Embodiment 31 provides the lipid nanoparticle of any one of embodiments 24-30, wherein the lipid nanoparticle comprises about 20-60% ionizable cationic lipid, about 5-25% helper lipid, about 25-55% sterol, and about 0.5-15% PEG-modified lipid.
Embodiment 32 provides a pharmaceutical composition comprising the lipid nanoparticle of any one of embodiments 24-31 and a pharmaceutically acceptable carrier or excipient.
Embodiment 33 provides a vaccine comprising one or more lipid nanoparticles of any one of embodiments 24-32 and/or the pharmaceutical composition of embodiment 21, and further comprising a pharmaceutically acceptable adjuvant.
Embodiment 34 provides a method of inducing in a subject an immune response to a monkeypox virus, comprising administering to the subject the vaccine of embodiment 33 in an amount effective to generate the immune response.
Embodiment 35 provides the method of embodiment 34, wherein the immune response comprises a T cell response and/or a B cell response.
Embodiment 36 provides the method of any one of embodiments 34-35, wherein the immune response comprises a neutralizing antibody response specific to the at least one monkeypox antigen protein.
Embodiment 37 provides the method of any one of embodiments 34-36, wherein the immune response inhibits infection by the monkeypox virus and/or replication of the monkeypox virus in the subject.
Embodiment 38 provides the method of any one of embodiments 34-37, wherein the subject is administered a single dose of the vaccine.
Embodiment 39 provides the method of any one of embodiments 34-38, wherein the subject is administered two or more doses of the vaccine, optionally wherein the two or more doses are administered 14-28 days apart.
Embodiment 40 provides the method of any one of embodiments 34-39, wherein each administration of the vaccine comprises a dose of mRNA of about 1 μg, 3 μg, 10 μg, 25 μg, 30 μg or 100 μg.
Embodiment 41 provides the method of any one of embodiments 34-40, wherein the effective amount is a total dose of about 1-500 μg, inclusive.
Embodiment 42 provides the method of any one of embodiments 34-41, wherein the vaccine is administered by intradermal injection, intramuscular injection, oral administration, intranasal administration, or intratracheal administration.
Embodiment 43 provides the method of any one of embodiments 34-42, wherein the subject has been exposed to, is infected with, or is at risk of infection by the monkeypox virus.
Embodiment 44 provides the method of any one of embodiments 34-43, wherein the subject is human.

### OTHER EMBODIMENTS

It is understood that the disclosed method and compositions are not limited to the particular methodology, protocols, and reagents described as these may vary. It is also to be understood that the terminology used herein is for the purpose of describing particular embodiments only, and is not intended to limit the scope of the present disclosure which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

1. An isolated messenger ribonucleic acid (mRNA) comprising a 5′ untranslated region (UTR), a 3′ UTR, and an open reading frame encoding at least one monkeypox (Mpox) virus antigen,
wherein the protein sequence of the at least one Mpox virus antigen comprises all or a portion of the protein sequence of a Mpox envelope or cell surface binding protein.

2. The isolated mRNA of claim 1, wherein the at least one Mpox virus antigen is selected from the group consisting of A29, E8L, M1R, A35R, B6R and any combination thereof.

3. The isolated mRNA of claim 1, wherein the amino acid sequences encoding two or more Mpox antigens are linked by independently selected linker sequences, optionally wherein the linker sequences comprise a 2A peptide sequence.

4. (canceled)

5. The isolated mRNA of claim 1, further comprising one or more transmembrane domain and cytosolic segments (TM/Cs).

6. The isolated mRNA of claim 5, wherein at least one of the following applies:
(a) the TM/C is selected from the group consisting of a HLA-CAAX, a Spike, an Env, a CAR, a CD86-TLR9, an E8L, an HLA, an HLA-EPM-EABR, an HLA-EABR-CAAX, a KEL, a FIBCD1, an ANPEP, an ASGR1, a CD38, a CD40LG, a CLEC2A, a CORIN, and a GLDN TM/C domain;
(b) the TM/C comprises an amino acid sequence selected from the group consisting of SEQ ID NOs: 11-19 and 28-36;
(c) the TM/C segment comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 11-19 and 28-36.

7-8. (canceled)

9. The isolated mRNA of claim 1, further comprising a signal peptide.

10. The isolated mRNA of claim 9, wherein at least one of the following applies:
(a) the signal peptide is selected from the group consisting of a CD8, a SARS COV2, a tPA, an IL2, an albumin, an Env, a Secrecon, and a IgKvIII signal peptide;
(b) the signal peptide is a CD8 signal peptide;
(c) the signal peptide comprises an amino acid sequence selected from the group consisting of SEQ ID NOs: 20-29;
(d) the signal peptide comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 20-29.

11-13. (canceled)

14. The isolated mRNA of claim 1, wherein the isolated mRNA further comprises a reporter gene, optionally wherein the reporter gene is GFP or a variant thereof.

15. (canceled)

16. The isolated mRNA of claim 1, wherein at least one of the following applies:
(a) the at least one antigen protein sequence comprises an amino acid sequence having 75%, 80%, 85%, 90%, 91%, 92%, 93%, 94%, 95%, 96%, 97%, 98%, 99%, 99.5% or more sequence identity to any one of SEQ ID NOs: 1-10;
(b) the at least one antigen protein sequence comprises an amino acid sequence set forth in SEQ ID NOs: 1-10.

17-18. (canceled)

19. The isolated mRNA of claim 1, wherein the mRNA encodes an amino acid sequence having 75%, 80%, 85%, 90%, 91%, 92%, 93%, 94%, 95%, 96%, 97%, 98%, 99%, 99.5% or more sequence identity to SEQ ID NO: 37, optionally wherein the mRNA encodes an amino acid sequence set forth in SEQ ID NO: 37.

20. An isolated polynucleotide encoding the mRNA of claim 1, optionally wherein the polynucleotide comprises one or more promoters or a polyadenylation signal operably linked to a sequence encoding the mRNA.

21. (canceled)

22. A vector comprising the polynucleotide of claim 20.

23. A method of producing a recombinant Mpox antigen protein, the method comprising
introducing into a host cell an isolated polynucleotide encoding the mRNA of claim 1, optionally wherein the polynucleotide comprises one or more promoters or a polyadenylation signal operably linked to a sequence encoding the mRNA, and
incubating the host cell under conditions sufficient for expression of the polynucleotide encoding the mRNA of claim 1, thereby producing the Mpox antigen protein.

24. A lipid nanoparticle comprising the mRNA of claim 1, optionally the molar ratio of lipid to mRNA is in the range of about 5:1 to 20:1, preferably 6:1.

25. (canceled)

26. The lipid nanoparticle of claim 24, wherein the lipid nanoparticle comprises at least one ionizable cationic lipid, at least one helper lipid, at least one sterol, or at least one PEG-modified lipid.

27. The lipid nanoparticle of claim 26, wherein at least one of the following applies:
(a) the at least one ionizable cationic lipid comprises 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), PNI ionizable lipid, SM-102, DLin-MC3-DMA, DLin-KC2-DMA, ALC-0315, or a combination thereof;
(b) the at least one helper lipid comprises 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC), 1,2-dioleoyl-sn-glycero-3-phosphocholine (DOPC), or a combination thereof;
(c) the at least one PEG-modified lipid comprises 1,2-dimyristoyl-racglycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, mPEG-DPPE, or a combination thereof;
(d) the at least one sterol is cholesterol;
(e) wherein the lipid nanoparticle comprises about 20-60% ionizable cationic lipid, about 5-25% helper lipid, about 25-55% sterol, and about 0.5-15% PEG-modified lipid.

28-31. (canceled)

32. A pharmaceutical composition comprising the lipid nanoparticle of claim 24 and a pharmaceutically acceptable carrier or excipient.

33. A vaccine comprising one or more lipid nanoparticles of claim 24, and further comprising a pharmaceutically acceptable adjuvant.

34. A method of inducing in a subject an immune response to a Mpox virus, comprising administering to the subject the vaccine of claim 33 in an amount effective to generate the immune response.

35. The method of claim 34, wherein at least one of the following applies:
(a) the immune response comprises a T cell response or a B cell response;
(b) the immune response comprises a neutralizing antibody response specific to the at least one Mpox antigen protein;
(c) the immune response inhibits infection by the Mpox virus or replication of the Mpox virus in the subject;
(d) the subject is administered a single dose of the vaccine;
(e) the subject is administered two or more doses of the vaccine, optionally wherein the two or more doses are administered 14-28 days apart;
(f) each administration of the vaccine comprises a dose of mRNA of about 1 μg, 3 μg, 10 μg, 25 μg, 30 μg or 100 μg;
(g) the effective amount is a total dose of about 1-500 μg, inclusive;
(h) the vaccine is administered by intradermal injection, intramuscular injection, oral administration, intranasal administration, or intratracheal administration;
(i) subject has been exposed to, is infected with, or is at risk of infection by Mpox virus;
(j) the subject is human.

36-44. (canceled)
